(19) United States
(12) Patent Application Publication
(10) Pub. No.: US 2025/0221996 A1
(43) Pub. Date: Jul. 10, 2025

(54) HETEROCYCLIC COMPOUNDS, COMPOSITIONS THEREOF, AND METHODS OF TREATMENT THEREWITH

(71) Applicant: BeiGene, Ltd., Grand Cayman (KY)

(72) Inventors: Ce WANG, Beijing (CN); Chao YU, Beijing (CN); Hao YUAN, Beijing (CN); Xiaoyu LI, Beijing (CN); Zhiwei WANG, Beijing (CN); Hanzi SUN, Beijing (CN)

(73) Assignee: BeiGene, Ltd., Grand Cayman (KY)

(21) Appl. No.: 19/011,372

(22) Filed: Jan. 6, 2025

Related U.S. Application Data
(63) Continuation of application No. PCT/CN2023/106299, filed on Jul. 7, 2023.

(30) Foreign Application Priority Data
Jul. 7, 2022 (WO) PCT/CN2022/104277

(51) Int. Cl. A61K 31/517; A61K 31/5386; C07D 487/08; C07D 498/08

(52) U.S. Cl. CPC A61K 31/517; A61K 31/5386; C07D 487/08; C07D 498/08

(57) ABSTRACT

Provided herein are compounds having the following structure:

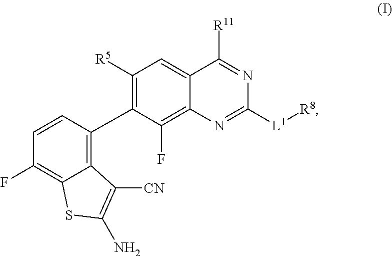

wherein the substituents are as defined herein, compositions comprising an effective amount of a compound, and methods for modulating activity of KRAS G12D.

### FIELD

Provided herein are heterocyclic compounds useful for treating cancer, a pharmaceutical composition comprising the compounds, and methods of using the compounds for treating cancer or a condition treatable or preventable by inhibition of KRAS G12D activity, comprising administering an effective amount of the compounds to a subject in need thereof.

### BACKGROUND

Ras is a family of proteins which are associated with cell membrane through their C-terminal membrane targeting region and well known as the molecular switch in intracellular signaling network (Cox A D, Der C J. Ras history: The saga continues. Small GTPases. 2010; 1(1):2-27). Ras proteins bind with either GTP or GDP and switch between “on” and “off” states. When Ras proteins bind with GDP, it is in the off (or inactive) state. And when Ras is switched on by certain growth promoting stimuli like growth factors, Ras proteins will be induced to exchange its bound GDP for a GTP and turn into on (or active) state (Malumbres M, Barbacid M. RAS oncogenes: the first 30 years. Nat Rev Cancer. 2003; 3(6):459-465). By switching to active state, Ras protein can interact with different downstream proteins and activate related signaling pathways (Berndt N, Hamilton A D, Sebti S M. Targeting protein prenylation for cancer therapy. Nat Rev Cancer. 2011; 11(11):775-791). Ras superfamily contains different subfamilies including Ras, Ral, Rap, Rheb, Rad, Rit and Miro (Wennerberg K, Rossman K L, Der C J. The Ras superfamily at a glance. J Cell Sci. 2005; 118(Pt 5):843-846). HRas, NRas and KRas are the most well studied proteins in Ras family since these proteins are the most common oncogenes in human cancers (O'Bryan J P. Pharmacological targeting of RAS: Recent success with direct inhibitors. Pharmacol Res. 2019; 139:503-511).

KRas is one of the most frequently mutated genes in human cancers. Based on data from Catalogue of Somatic Mutations (COSMIC) database, KRas mutation can be found in about 20% of human cancers, including pancreatic cancer, colorectal cancer, lung cancer, skin cancer etc. (O'Bryan J P. Pharmacological targeting of RAS: Recent success with direct inhibitors. Pharmacol Res. 2019; 139:503-511). The most common KRas mutations are found at position G12 and G13 by blocking the GTPase activating proteins (GAP) stimulated GTP hydrolysis activity of KRas (Wang W, Fang G, Rudolph J. Ras inhibition via direct Ras binding—is there a path forward?. Bioorg Med Chem Lett. 2012; 22(18):5766-5776). That results in the over activation of KRas protein and ultimately leads to uncontrolled cell proliferation and cancer.

Among different cancers, pancreatic cancer is considered as the most KRas-addicted cancer type. KRas mutation is found in 94.1% of pancreatic ductal adenocarcinoma (PDAC). G12D (41%) and G12V (34%) mutations of KRas are the two most predominant mutations in all the KRas mutated PDAC (Waters A M, Der C J. KRAS: The Critical Driver and Therapeutic Target for Pancreatic Cancer. Cold Spring Harb Perspect Med. 2018; 8(9):a031435). In vivo data generated by mouse models proves that the progression and maintenance of pancreatic cancer highly rely on the constitutive activation of KRas downstream signaling (Siveke J T, Schmid R M. Chromosomal instability in mouse metastatic pancreatic cancer—it's Kras and Tp53 after all. Cancer Cell. 2005; 7(5):405-407). This indicates that mutated KRas protein is a highly attractive drug target for pancreatic cancer and also other cancers with KRas mutation. Because Kras protein is generally considered as a non-druggable target, there is no therapeutics which can selectively target Kras protein with G12D mutation in the clinic.

Thus, KRas G12D mutation is a highly attractive target for pancreatic cancer and other cancers with this mutation. As such, small-molecule therapeutic agents that are capable of selectively binding with Kras G12D and inhibiting its function would be very useful.

Citation or identification of any reference in this section of this application is not to be construed as an admission that the reference is prior art to the present application.

### SUMMARY

Provided herein are compounds having the following formula (I):

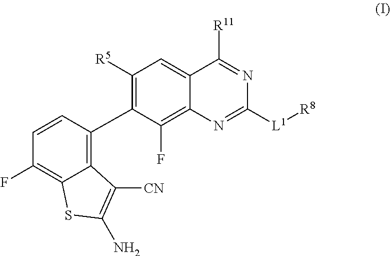

and pharmaceutically acceptable salts, tautomers, stereoisomers, enantiomers, atropisomers, isotopologues, and prodrugs thereof, wherein the substituents are as defined herein. In one embodiment, the compound is not any one of the compounds in Table 1.

In one embodiment, the compound is selected from Table 2.

In one embodiment, provided herein is a method for inhibiting the activity of KRAS mutant protein in a cell, comprising contacting said cell with an effective amount of a compound provided herein, or a pharmaceutically acceptable salt, tautomer, isotopologue, stereoisomer, or prodrug thereof, optionally wherein the KRAS mutant protein is KRAS G12D mutant protein.

In one embodiment, provided herein is a method for treatment or prevention of cancer, the method comprising administering to a subject in need thereof an effective amount of a compound provided herein, or a pharmaceutically acceptable salt, tautomer, isotopologue, stereoisomer, or prodrug thereof, optionally wherein the cancer is mediated by KRAS mutation; preferably KRAS G12D mutation.

### DETAILED DESCRIPTION

### Definitions

As used herein, “KRAS gene” refers to a gene selected from the group consisting of: DIRAS1; DIRAS2; DIRAS3; ERAS; GEM; HRAS; KRAS; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS; RRAS2, and mutants thereof.

As used herein, “KRAS protein” refers to a protein or an isoform thereof expressed by a KRAS gene (Scolnick E M, Papageoege A G, Shih T Y (1979), “Guanine nucleotide-binding activity for src protein of rat-derived murine sarcoma viruses,” Proc Natl Acad Sci USA. 76 (5): 5355-5559; Kranenburg O (November 2005) “The KRAS oncogene: past, present, and future,” Biochimica et Biophysica Acta (BBA)—Reviews on Cancer, 1756 (2): 81-2).

As used herein, “G12D mutation” refers to the mutation of the 12^{th} amino acid residue located in the G domain of KRAS protein from glycine to aspartic acid.

As used herein, “KRAS G12D” or “G12D” refer to KRAS protein with G12D mutation.

As used herein, and in the specification and the accompanying claims, the indefinite articles “a” and “an” and the definite article “the” include plural as well as single referents, unless the context clearly indicates otherwise.

As used herein, and unless otherwise specified, the terms “about” and “approximately,” when used in connection with doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. In certain embodiments, the terms “about” and “approximately,” when used in this context, contemplate a dose, amount, or weight percent within 30%, within 20%, within 15%, within 10%, or within 5%, of the specified dose, amount, or weight percent.

As used herein, and unless otherwise specified, the terms “about” and “approximately,” when used in connection with a numeric value or range of values which is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or XRPD; indicate that the value or range of values may deviate to an extent deemed reasonable to one of ordinary skill in the art while still describing the solid form. Techniques for characterizing crystal forms and amorphous solids include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility studies, and dissolution studies. In certain embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5%, 1%, 0.5%, or 0.25% of the recited value or range of values. For example, in some embodiments, the value of an XRPD peak position may vary by up to +0.2° 20 (or +0.2 degree 20) while still describing the particular XRPD peak.

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 or carbon atoms. Representative alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₇CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

A “cycloalkyl” group is a saturated, partially saturated, or unsaturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. A cycloalkyl comprising more than one ring may be fused, spiro, or bridged, or combinations thereof. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. A heterocyclyl group may include multiple condensed rings including, but are not limited to, bicyclic, tricyclic, and quadracyclic rings, as well as bridged or spirocyclic ring systems. Heterocyclyl groups encompass unsaturated, partially saturated and saturated ring systems, such as, for example, imidazolyl, imidazolinyl and imidazolidinyl (e.g., imidazolidin-4-one or imidazolidin-2,4-dionyl) groups. The phrase heterocyclyl includes fused ring species, including those comprising fused aromatic and non-aromatic groups, such as, for example, 1- and 2-aminotetraline, benzotriazolyl (e.g., 1H-benzo[d][1,2,3]triazolyl), benzimidazolyl (e.g., 1H-benzo[d]imidazolyl), 2,3-dihydrobenzo[1,4]dioxinyl, and benzo[1,3]dioxolyl. The phrase also includes bridged polycyclic ring systems containing a heteroatom such as, but not limited to, quinuclidyl. Representative examples of a heterocyclyl group include, but are not limited to, aziridinyl, azetidinyl, azepanyl, oxetanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, benzisoxazolyl (e.g., benzo[d]isoxazolyl), thiazolyl, thiazolinyl, isothiazolyl, thiadiazolyl, oxadiazolyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dioxyl, dithianyl, pyranyl, pyridyl, pyrimidyl, pyridazinyl, pyrazinyl, triazinyl, dihydropyridyl, dihydrodithiinyl, dihydrodithionyl, 1,4-dioxaspiro[4.5]decanyl, 2-oxo-1-oxa-3,8-diazaspiro[4.5]decane, 1-oxo-2,8-diazaspiro[4.5]decane, 3-oxo-2,8-diazaspiro[4.5]decane, 3-oxo-1-oxa-4,9-diazaspiro[5.5]undecane, 2-oxo-1-oxa-3,9-diazaspiro[5.5]undecane, homopiperazinyl, quinuclidyl, indolyl (e.g., indolyl-2-onyl or isoindolin-1-onyl), indolinyl, isoindolyl, isoindolinyl, azaindolyl (pyrrolopyridyl or 1H-pyrrolo[2,3-b]pyridyl), indazolyl, indolizinyl, benzotriazolyl (e.g., 1H-benzo[d][1,2,3]triazolyl), benzimidazolyl (e.g., 1H-benzo[d]imidazolyl or 1H-benzo[d]imidazol-2(3H)-onyl), benzofuranyl, benzothiophenyl, benzothiazolyl, benzoxadiazolyl, benzoxazinyl, benzodithiinyl, benzoxathiinyl, benzothiazinyl, benzoxazolyl (i.e., benzo[d]oxazolyl), benzothiazolyl, benzothiadiazolyl, benzo[1,3]dioxolyl, pyrazolopyridyl (for example, 1H-pyrazolo[3,4-b]pyridyl, 1H-pyrazolo[4,3-b]pyridyl), imidazopyridyl (e.g., azabenzimidazolyl or 1H-imidazo[4,5-b]pyridyl), triazolopyridyl, isoxazolopyridyl, purinyl, xanthinyl, adeninyl, guaninyl, quinolinyl, isoquinolinyl (e.g., 3,4-dihydroisoquinolin-1(2H)-onyl), quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, tetrahydropyrimidin-2(1H)-one and tetrahydroquinolinyl groups. Representative non-aromatic heterocyclyl groups do not include fused ring species that comprise a fused aromatic group. Examples of non-aromatic heterocyclyl groups include aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, or tetrahydropyrimidin-2(1H)-one. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “heteroaryl” group is an aryl ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1H-pyrrolo[2,3-b]pyridyl), indazolyl, benzimidazolyl (e.g., 1H-benzo[d]imidazolyl), imidazopyridyl (e.g., azabenzimidazolyl or 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4-dihydroisoquinolin-1(2H)-onyl), tetrahydroquinolinyl, quinoxalinyl, and quinazolinyl groups.

As used herein, “spirocyclic ring” refers to two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings.

A “cycloalkylalkyl” group is a radical of the formula: -alkyl-cycloalkyl, wherein alkyl and cycloalkyl are as defined above. Substituted cycloalkylalkyl groups may be substituted at the alkyl, the cycloalkyl, or both the alkyl and the cycloalkyl portions of the group. Representative cycloalkylalkyl groups include but are not limited to methylcyclopropyl, methylcyclobutyl, methylcyclopentyl, methylcyclohexyl, ethylcyclopropyl, ethylcyclobutyl, ethylcyclopentyl, ethylcyclohexyl, propylcyclopentyl, propylcyclohexyl and the like.

An “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and fused (cycloalkylaryl)alkyl groups such as 4-ethyl-indanyl.

An “heterocyclylalkyl” group is a radical of the formula: -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocylylalkyl groups include but are not limited to 4-ethyl-morpholinyl, 4-propylmorpholinyl, furan-2-yl methyl, furan-3-yl methyl, pyridin-3-yl methyl, tetrahydrofuran-2-yl ethyl, and indol-2-yl propyl.

A “halogen” is fluorine, chlorine, bromine or iodine.

A “hydroxyalkyl” group is an alkyl group as described above substituted with one or more hydroxy groups.

An “alkoxy” or “alkoxyl” group is —O-(alkyl), wherein alkyl is defined above.

An “alkoxyalkyl” group is -(alkyl)-O-(alkyl), wherein alkyl is defined above.

An “amino” group is a radical of the formula: —NH₂.

An “alkylamino” group is a radical of the formula: —NH-alkyl or —N(alkyl)₂, wherein each alkyl is independently as defined above.

A “carboxy” group is a radical of the formula: —C(O)OH.

An “aminocarbonyl” group is a radical of the formula: —C(O)N(R^{#})₂, —C(O)NH(R^{#}) or —C(O)NH₂, wherein each R⁸ is independently a substituted or unsubstituted alkyl, cycloalkyl, aryl, aralkyl, heterocyclyl or heterocyclyl group as defined herein.

An “acylamino” group is a radical of the formula: —NHC(O)(R^{#}) or —N(alkyl)C(O)(R^{#}), wherein each alkyl and R⁸ are independently as defined above.

A “sulfonylamino” group is a radical of the formula: —NHSO₂(R^{#}) or —N(alkyl)SO₂(R^{#}), wherein each alkyl and R¹¹ are defined above.

A “urea” group is a radical of the formula: —N(alkyl)C(O)N(R^{#})₂, —N(alkyl)C(O)NH(R^{#}), N(alkyl)C(O)NH₂, —NHC(O)N(R^{#})₂, —NHC(O)NH(R^{#}), or —NH(CO)NHR^{#}, wherein each alkyl and R⁸ are independently as defined above.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds of formula (I) include, but are not limited to those well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof.

The use of stereomerically pure forms of such compounds, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN, 1972).

It should also be noted the compounds can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

As used herein and unless otherwise indicated, “atropisomers” refer to stereoisomers resulting from hindered rotation about a single bond axis where the rotational barrier is high enough to allow for the isolation of the individual rotational isomers

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

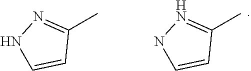

As readily understood by one skilled in the art, a wide variety of functional groups and other structures may exhibit tautomerism and all tautomers of compounds of formula (I) are within the scope of the present invention.

It should also be noted the compounds can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds.

“Treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In some embodiments, “treating” means an alleviation, in whole or in part, of a disorder, disease or condition, or a slowing, or halting of further progression or worsening of those symptoms. In another embodiment, “treating” means and alleviation, in whole or in part, of a disorder, disease or condition, or symptoms associated with a condition, wherein the condition is treatable or preventable by inhibition of KRAS G12D.

“Preventing” as used herein, means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition. In one embodiment, the condition is a condition, treatable or preventable by inhibition of KRAS G12D.

The term “effective amount” in connection with a compound means an amount capable of treating or preventing a disorder, disease or condition, or symptoms thereof, disclosed herein.

The term “subject” includes an animal, including, but not limited to, an animal such a cow, monkey, horse, sheep, pig, chicken, turkey, quail, cat, dog, mouse, rat, rabbit or guinea pig, in one embodiment a mammal, in another embodiment a human.

### Compounds

Provided herein are compounds having the following formula (I):

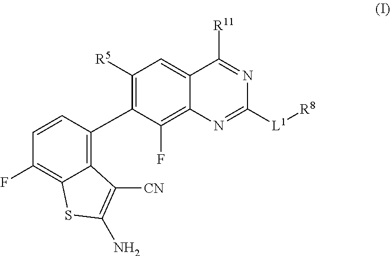

and pharmaceutically acceptable salts, tautomers, stereoisomers, enantiomers, atropisomers, isotopologues, and prodrugs thereof,
wherein:
R⁵ is H, —Cl, —CF₃, or unsubstituted C₁₋₄alkyl;
R⁸ is unsubstituted or substituted alkyl, unsubstituted or substituted cycloalkyl, unsubstituted or substituted cycloalkylalkyl, unsubstituted or substituted aryl, unsubstituted or substituted aralkyl, unsubstituted or substituted heterocyclyl, unsubstituted or substituted heterocyclylalkyl, unsubstituted or substituted heteroaryl, unsubstituted or substituted heteroaralkyl, unsubstituted or substituted spirocyclic ring, or unsubstituted or substituted -alkyl-spirocyclic ring;
R¹¹ is 3,8-diazabicyclo[3.2.1]oct-3-yl,

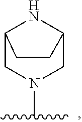 [SMILES: *N1CC2CCC(C1)N2]

3,8-diazabicyclo[3.2.1]oct-8-yl,

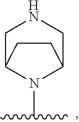 [SMILES: *N1C2CCC1CNC2]

3-oxa-7,9-diazabicyclo[3.3.1]non-9-yl,

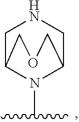 [SMILES: *N1C2CNCC1COC2]

3-oxa-7,9-diazabicyclo[3.3.1]non-7-yl, or

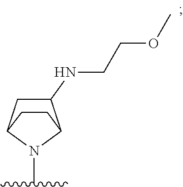 [SMILES: *N1C2CCC1C(NCCOC)C2]

and
L¹ is —O— or a bond.

In one embodiment, the compound is not any one of the compounds in Table 1.

TABLE 1

| | |
|---|---|
| 4-(4-(3,8-diazabicyclo[3.2.1]octan-3-yl)-8-fluoro-2- (((2R, 7aS)-2-fluorotetrahydro-1H-pyrrolizin-7a(5H)- yl)methoxy)-6-(trifluoromethyl)quinazolin-7-yl)-2-amino- 7-fluorobenzo[b]thiophene-3-carbonitrile | 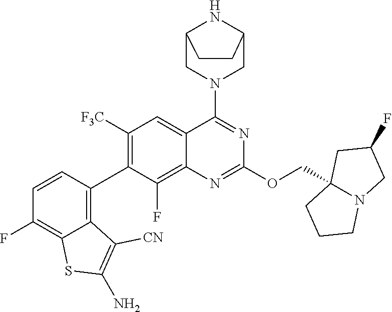 [SMILES: N#Cc1c(N)sc2c(F)ccc(-c3c(C(F)(F)F)cc4c(N5CC6CCC(C5)N6)nc(OC[C@@]56CCCN5C[C@H](F)C6)nc4c3F)c12] |
| 4-(4-(3,8-diazabicyclo[3.2.1]octan-3-yl)-6-chloro-8- fluoro-2-(((2R, 7aS)-2-fluorotetrahydro-1H-pyrrolizin- 7a(5H)-yl)methoxy)quinazolin-7-yl)-2-amino-7- fluorobenzo[b]thiophene-3-carbonitrile | 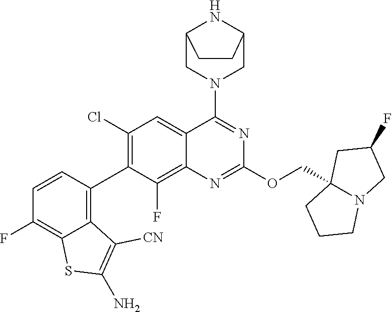 [SMILES: N#Cc1c(N)sc2c(F)ccc(-c3c(Cl)cc4c(N5CC6CCC(C5)N6)nc(OC[C@@]56CCCN5C[C@H](F)C6)nc4c3F)c12] |
| 4-(4-(3,8-diazabicyclo[3.2.1]octan-3-yl)-8-fluoro-2- (((2S,4R)-4-fluoro-1-methylpyrrolidin-2-yl)methoxy)-6- (trifluoromethyl)quinazolin-7-yl)-2-amino-7-fluoro benzo[b]thiophene-3-carbonitrile | 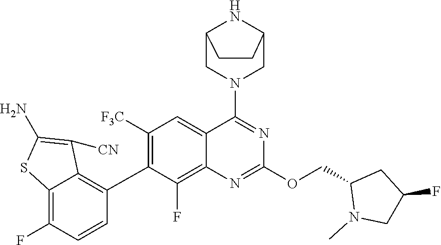 [SMILES: CN1C[C@H](F)C[C@H]1COc1nc(N2CC3CCC(C2)N3)c2cc(C(F)(F)F)c(-c3ccc(F)c4sc(N)c(C#N)c34)c(F)c2n1] |
| 8-((2S)-2-((7-(2-amino-3-cyano-7- fluorobenzo[b]thiophen-4-yl)-4-(3,8- diazabicyclo[3.2.1]octan-3-yl)-6-chloro-8- fluoroquinazolin-2-yl)oxy)propyl)-8-azabicyclo [3.2.1]octane-3-carbonitrile | 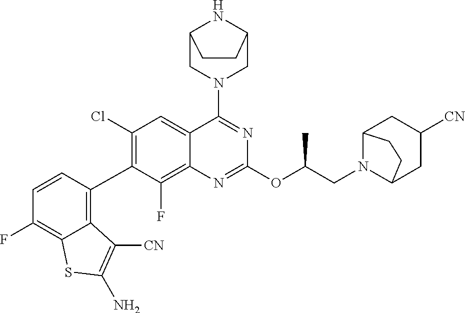 [SMILES: C[C@@H](CN1C2CCC1CC(C#N)C2)Oc1nc(N2CC3CCC(C2)N3)c2cc(Cl)c(-c3ccc(F)c4sc(N)c(C#N)c34)c(F)c2n1] |
| 4-(4-(3,8-diazabicyclo[3.2.1]octan-3-yl)-6-chloro-8- fluoro-2-(((S)-1-(4-fluoropiperidin-1-yl)propan-2- yl)oxy)quinazolin-7-yl)-2-amino-7- fluorobenzo[b]thiophene-3-carbonitrile | 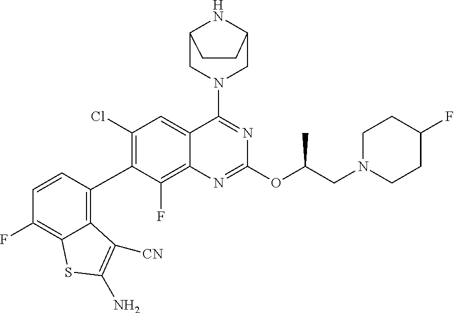 [SMILES: C[C@@H](CN1CCC(F)CC1)Oc1nc(N2CC3CCC(C2)N3)c2cc(Cl)c(-c3ccc(F)c4sc(N)c(C#N)c34)c(F)c2n1] |
| 4-(4-(3,8-diazabicyclo[3.2.1]octan-3-yl)-6-chloro-8- fluoro-2-((1-((3-fluoro pyrrolidin-1- yl)methyl)cyclobutyl)methoxy)quinazolin-7-yl)-2-amino- 7-fluorobenzo [b]thiophene-3-carbonitrile | 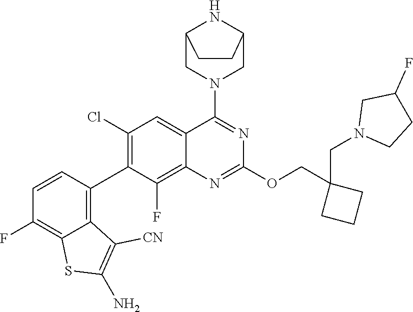 [SMILES: N#Cc1c(N)sc2c(F)ccc(-c3c(Cl)cc4c(N5CC6CCC(C5)N6)nc(OCC5(CN6CCC(F)C6)CCC5)nc4c3F)c12] |
| 4-(4-(3,8-diazabicyclo[3.2.1]octan-3-yl)-8-fluoro-2-((1- methyl-2-oxabicyclo[2.1.1]hexan-4-yl)methoxy)-6- (trifluoromethyl)quinazolin-7-yl)-2-amino-7-fluoro benzo[b]thiophene-3-carbonitrile | 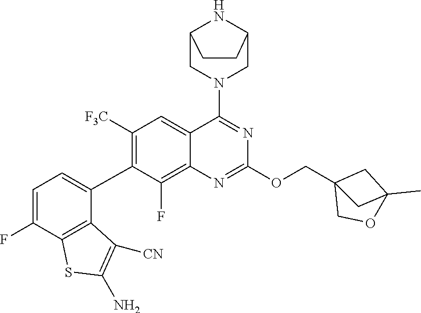 [SMILES: CC12CC(COc3nc(N4CC5CCC(C4)N5)c4cc(C(F)(F)F)c(-c5ccc(F)c6sc(N)c(C#N)c56)c(F)c4n3)(CO1)C2] |
| 4-(2-((2-oxabicyclo[2.1.1]hexan-4-yl)methoxy)-4-(3,8- diaza bicyclo[3.2.1]octan-3-yl)-8-fluoro-6- (trifluoromethyl)quinazolin-7-yl)-2-amino-7-fluorobenzo [b]thiophene-3-carbonitrile | 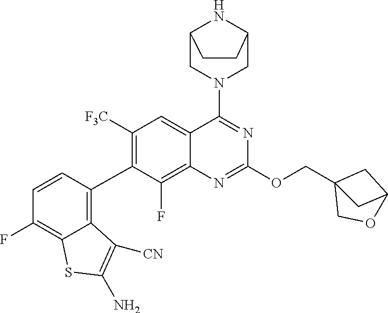 [SMILES: N#Cc1c(N)sc2c(F)ccc(-c3c(C(F)(F)F)cc4c(N5CC6CCC(C5)N6)nc(OCC56COC(C5)C6)nc4c3F)c12] |

In one embodiment, L¹ is —O—.

In one embodiment, R⁵ is —CF₃;

R¹¹ is

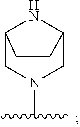 [SMILES: *N1CC2CCC(C1)N2]

R⁸ is unsubstituted or substituted heterocyclylalkyl.

In one embodiment, the heterocyclylalkyl comprises at least one oxygen as the ring member in the heterocyclyl moiety.

In one embodiment, R⁸ is -L^{8a}-R^{8a};
L^{8a} is —(CH₂)ₙ—;
n is an integer of 1, 2, or 3;
R^{8a} is oxetanyl, tetrahydrofuryl, tetrahydro-2H-pyranyl, dihydro-2H-pyranyl, oxabicyclo[2.1.1]hexyl, oxabicyclo[2.2.1]heptyl, oxaspiro[3.3]heptyl, oxabicyclo[3.2.1]octyl, oxabicyclo[2.2.2]octyl, oxaspiro[3.5]nonyl, or oxaspiro[3.4]octyl;
R^{8a} is optionally substituted.

In some embodiment, R^{8a} is optionally substituted with halogen, cyano, hydroxy, alkoxy, or alkyl optionally substituted with halogen, cyano, hydroxy, alkoxy, cycloalkyl or cycloalkyloxy.

In one embodiment, R⁸ is

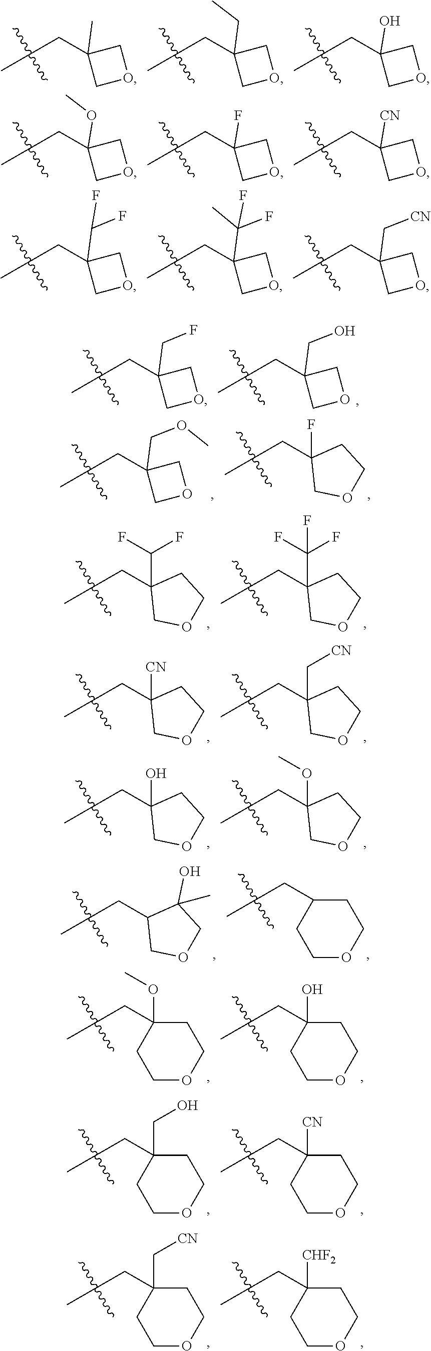

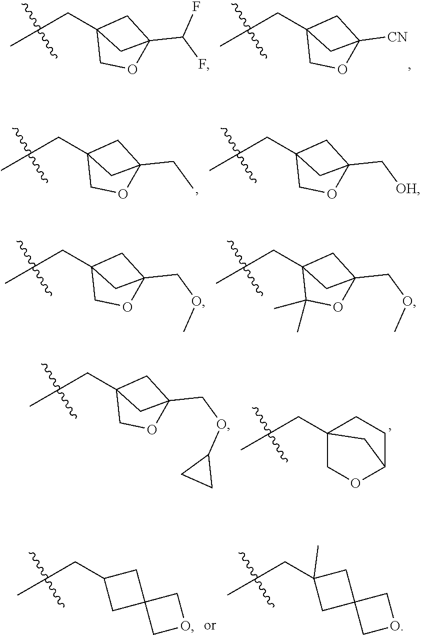

In one embodiment, the heterocyclylalkyl comprises at least one nitrogen as the ring member in the heterocyclyl moiety.

In one embodiment, R⁸ is -L^{8a}-R^{8a};
L^{8a} is —(CH₂)ₙ—;
n is an integer of 1, 2, or 3;
R^{8a} is azetidyl, pyridyl, isoxazolyl, oxazolyl, dihydro-2H-pyranyl, tetrahydro-2H-pyranyl, pyrrolidinonyl, azaspiro[3.3]heptyl, azabicyclo[2.1.1]hexyl, pyrrolidyl, 1H-pyrazolyl; and
R^{8a} is optionally substituted.

In some embodiment, R^{8a} is optionally substituted with halogen, cyano, hydroxy, alkoxy, or alkyl optionally substituted with halogen, cyano, hydroxy, or alkoxy.

In one embodiment. R⁸ is

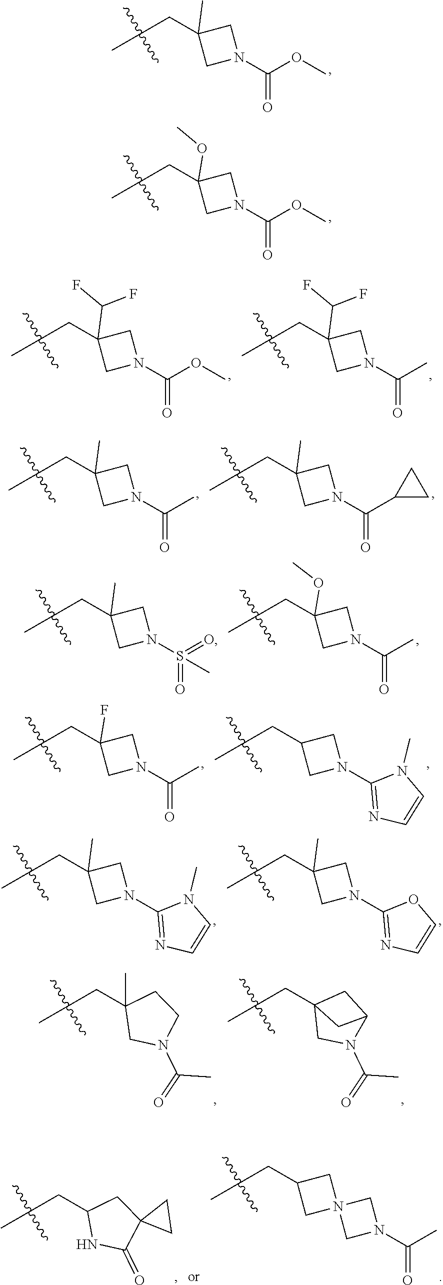

In one embodiment, L¹ is a bond;
R⁵ is —CF₃;
R¹¹ is

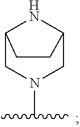 [SMILES: *N1CC2CCC(C1)N2]

R⁸ is unsubstituted or substituted heterocyclyl, unsubstituted or substituted heterocyclylalkyl, unsubstituted or substituted cycloalkyl, or unsubstituted or substituted cycloalkylalkyl.

In one embodiment, R⁸ is -L^{8a}-R^{8a};
L^{8a} is —(CH₂)ₙ—;
n is an integer of 0, 1, 2, or 3;
R^{8a} is cyclopropyl, cyclobutyl, cyclopentyl, cyclohexyl, cycloheptyl, or azetidyl; and
R^{8a} is optionally substituted.
   wherein:
   R⁵ is H, —Cl, —CF₃, or unsubstituted C₁₋₄alkyl;
   R⁸ is unsubstituted or substituted alkyl, unsubstituted or substituted cycloalkyl, unsubstituted or substituted cycloalkylalkyl, unsubstituted or substituted aryl, unsubstituted or substituted aralkyl, unsubstituted or substituted heterocyclyl, unsubstituted or substituted heterocyclylalkyl, unsubstituted or substituted heteroaryl, unsubstituted or substituted heteroaralkyl, unsubstituted or substituted spirocyclic ring, or unsubstituted or substituted -alkyl-spirocyclic ring;
   R¹¹ is 3,8-diazabicyclo[3.2.1]oct-3-yl,

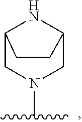 [SMILES: *N1CC2CCC(C1)N2]

3,8-diazabicyclo[3.2.1]oct-8-yl,

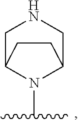 [SMILES: *N1C2CCC1CNC2]

3-oxa-7,9-diazabicyclo[3.3.1]non-9-yl,

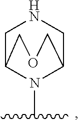 [SMILES: *N1C2CNCC1COC2]

3-oxa-7,9-diazabicyclo[3.3.1]non-7-yl, or

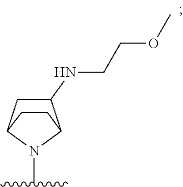 [SMILES: *N1C2CCC1C(NCCOC)C2]

and
L¹ is —O— or a bond.

In one embodiment, L¹ is —O—.

In one embodiment, R⁵ is —CF₃;
R¹¹ is

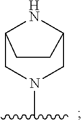 [SMILES: *N1CC2CCC(C1)N2]

R⁸ is unsubstituted or substituted heterocyclylalkyl.

In one embodiment, the heterocyclylalkyl comprises at least one oxygen as the ring member in the heterocyclyl moiety.

In one embodiment, R⁸ is -L^{8a}-R^{8a};
L^{8a} is —(CH₂)ₙ—;
n is an integer of 1, 2, or 3;
R^{8a} is oxetanyl, tetrahydrofuryl, tetrahydro-2H-pyranyl, dihydro-2H-pyranyl, oxabicyclo[2.1.1]hexyl, oxabicyclo[2.2.1]heptyl, oxaspiro[3.3]heptyl, oxabicyclo[3.2.1]octyl, oxabicyclo[2.2.2]octyl, oxaspiro[3.5]nonyl, or oxaspiro[3.4]octyl;
R^{8a} is optionally substituted.

In one embodiment, R^{8a} is

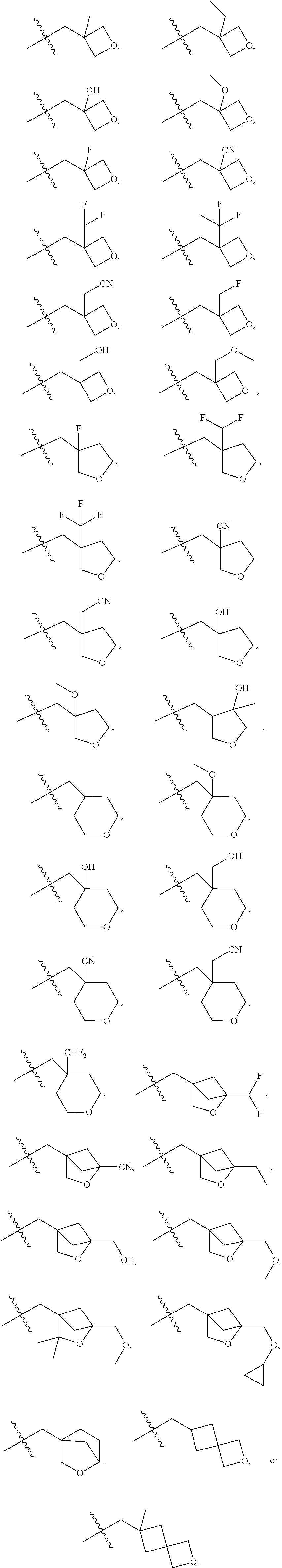

In one embodiment, the heterocyclylalkyl comprises at least one nitrogen as the ring member in the heterocyclyl moiety.

In one embodiment, R⁸ is -L^{8a}-R^{8a};
L^{8a} is —(CH₂)ₙ;
n is an integer of 1, 2, or 3;
R^{8a} is azetidyl, pyridyl, isoxazolyl, oxazolyl, dihydro-2H-pyranyl, tetrahydro-2H-pyranyl, pyrrolidinonyl, azaspiro[3.3]heptyl, azabicyclo[2.1.1]hexyl, pyrrolidyl, 1H-pyrazolyl; and
R^{8a} is optionally substituted.

In one embodiment, R⁸ is

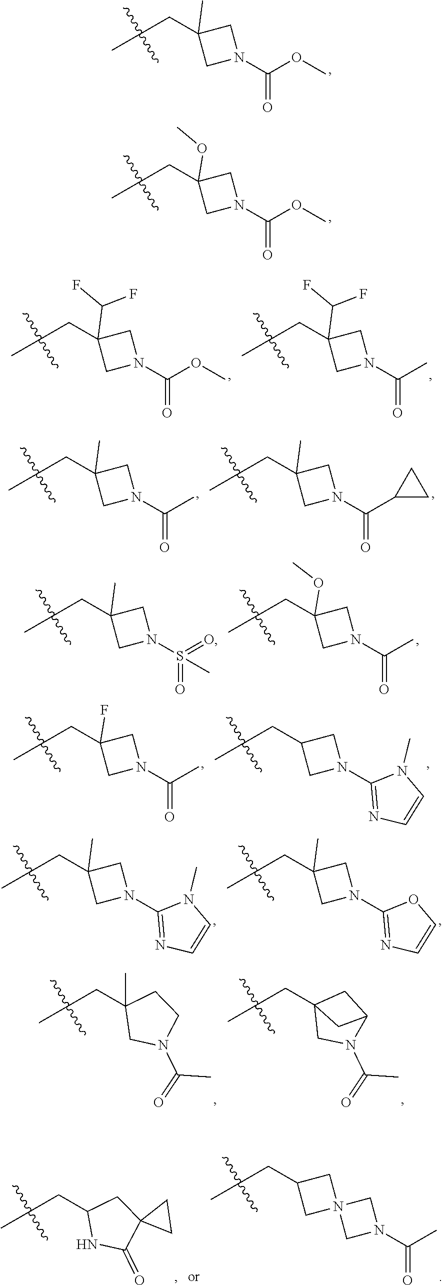

In one embodiment, L¹ is a bond;
R⁵ is —CF₃;
R¹¹ is

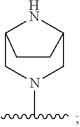 [SMILES: *N1CC2CCC(C1)N2]

R^{8a} is unsubstituted or substituted heterocyclyl, unsubstituted or substituted heterocyclylalkyl, unsubstituted or substituted cycloalkyl, or unsubstituted or substituted cycloalkylalkyl.

In one embodiment, R⁸ is -L^{8a}-R^{8a};
L^{8a} is —(CH₂)ₙ;
n is an integer of 0, 1, 2, or 3;
R^{8a} is cyclopropyl, cyclobutyl, cyclopentyl, cyclohexyl, cycloheptyl, or azetidyl; and
R^{8a} is optionally substituted.

In one embodiment, R⁸ is

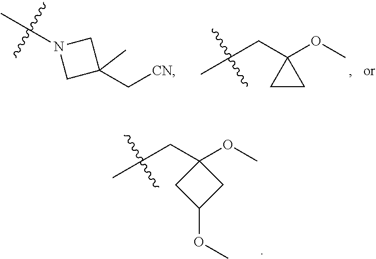

In one embodiment, R⁵ is —CF₃;
L¹ is —O—;
R¹¹ is 3,8-diazabicyclo[3.2.1]oct-8-yl,

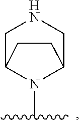 [SMILES: *N1C2CCC1CNC2]

3-oxa-7,9-diazabicyclo[3.3.1]non-9-yl,

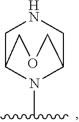 [SMILES: *N1C2CNCC1COC2]

3-oxa-7,9-diazabicyclo[3.3.1]non-7-yl, or

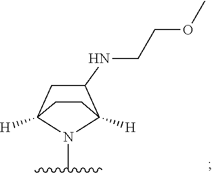 [SMILES: *N1[C@@H]2CC[C@H]1C(NCCOC)C2]

R⁸ is

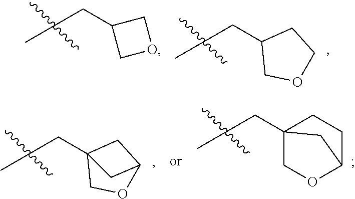

and
R⁸ is optionally substituted.

In one embodiment, the compound is

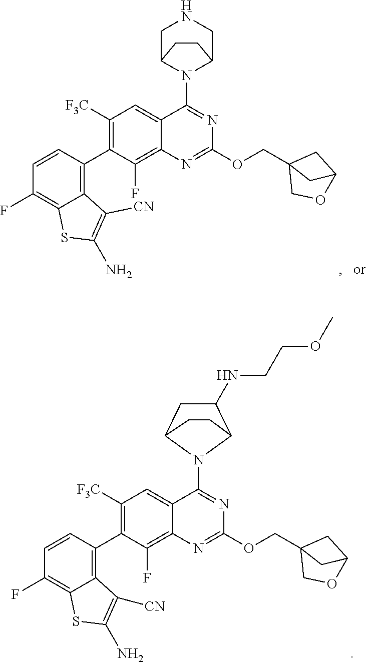 [SMILES: N#Cc1c(N)sc2c(F)ccc(-c3c(C(F)(F)F)cc4c(N5C6CCC5CNC6)nc(OCC56COC(C5)C6)nc4c3F)c12 ; COCCNC1CC2CCC1N2c1nc(OCC23COC(C2)C3)nc2c(F)c(-c3ccc(F)c4sc(N)c(C#N)c34)c(C(F)(F)F)cc12]

In one embodiment, R⁵ is —Cl;
L¹ is —O—;
R⁸ is

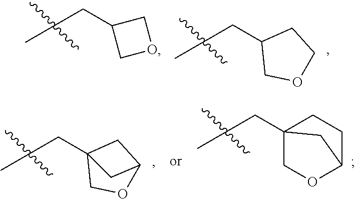

and
R⁸ is optionally substituted.

In one embodiment, the compound is

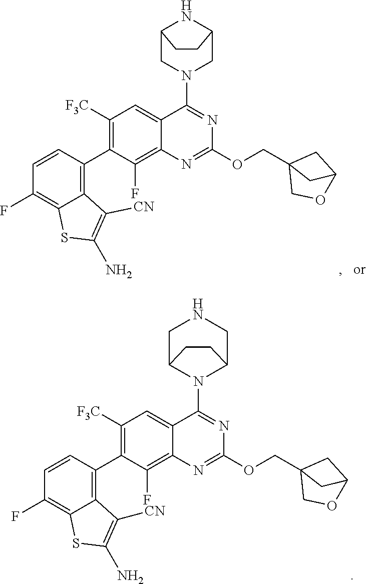 [SMILES: N#Cc1c(N)sc2c(F)ccc(-c3c(C(F)(F)F)cc4c(N5CC6CCC(C5)N6)nc(OCC56COC(C5)C6)nc4c3F)c12 ; N#Cc1c(N)sc2c(F)ccc(-c3c(C(F)(F)F)cc4c(N5C6CCC5CNC6)nc(OCC56COC(C5)C6)nc4c3F)c12]

In one embodiment, R⁵ is unsubstituted C₁₋₄alkyl;
R¹¹ is

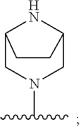 [SMILES: *N1CC2CCC(C1)N2]

### L¹ is —O—;

R⁸ is

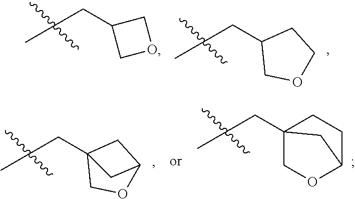

and
R⁸ is optionally substituted.

In one embodiment, the compound is

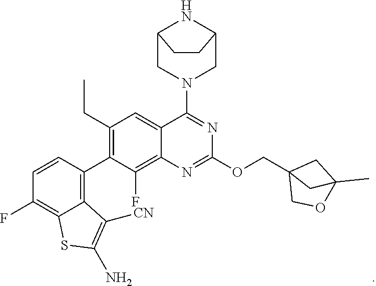 [SMILES: CCc1cc2c(N3CC4CCC(C3)N4)nc(OCC34COC(C)(C3)C4)nc2c(F)c1-c1ccc(F)c2sc(N)c(C#N)c12]

In one embodiment, R⁵ is —CF₃;
R¹¹ is 3-oxa-7,9-diazabicyclo[3.3.1]non-9-yl,

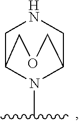 [SMILES: *N1C2CNCC1COC2]

3-oxa-7,9-diazabicyclo[3.3.1]non-7-yl;
L¹ is —O—;
R⁸ is

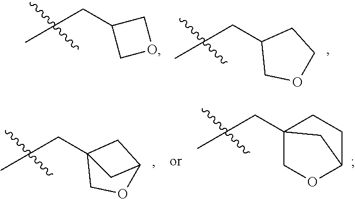

R⁸ is optionally substituted.

In one embodiment, the compound is

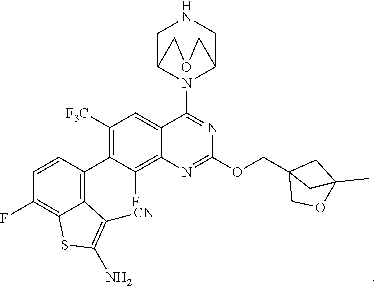 [SMILES: CC12CC(COc3nc(N4C5CNCC4COC5)c4cc(C(F)(F)F)c(-c5ccc(F)c6sc(N)c(C#N)c56)c(F)c4n3)(CO1)C2]

Aspect 2: In one embodiment, the compound is selected from Table 2.

Aspect 3: In one embodiment, provided herein is a pharmaceutical composition comprising an effective amount of a compound provided herein, or a pharmaceutically acceptable salt, tautomer, isotopologue, stereoisomer, or prodrug thereof, and a pharmaceutically acceptable carrier, excipient or vehicle.

Aspect 4: In one embodiment, provided herein is a method for inhibiting the activity of KRAS mutant protein in a cell, comprising contacting said cell with an effective amount of a compound provided herein, or a pharmaceutically acceptable salt, tautomer, isotopologue, stereoisomer, or prodrug thereof, optionally wherein the KRAS mutant protein is KRAS GI2D mutant protein.

Aspect 5: In one embodiment, provided herein is a method for treatment or prevention of cancer, the method comprising administering to a subject in need thereof an effective amount of a compound provided herein, or a pharmaceutically acceptable salt, tautomer, isotopologue, stereoisomer, or prodrug thereof, optionally wherein the cancer is mediated by KRAS mutation; preferably KRAS G12D mutation.

Aspect 6: Provided here is a method of modulating activity of KRAS GI2D, comprising contacting said cell with an effective amount of a compound provided herein, or a pharmaceutically acceptable salt, tautomer, isotopologue, stereoisomer, or prodrug thereof.

Aspect 7: Provided here is a method for the treatment or prevention of a cancer, the methods comprising administering to a subject in need thereof an effective amount of a compound provided herein.

Aspect 8: Provided herein is a kit for treating cancer, the kit comprising (a) a pharmaceutical composition comprising a compound provided herein; and (b) instructions for administration of an effective amount of the pharmaceutical composition comprising the KRAS G12D inhibitor provided herein to treat cancer in an individual.

The present embodiments can be understood more fully by reference to the detailed description and examples, which are intended to exemplify non-limiting embodiments.

### Methods for Making Compounds

The Compounds can be made using conventional organic syntheses and commercially available starting materials. By way of example and not limitation, Compounds of formula (I) can be prepared as outlined in Schemes 1-3 shown below as well as in the examples set forth herein. It should be noted that one skilled in the art would know how to modify the procedures set forth in the illustrative schemes and examples to arrive at the desired products. Common protecting groups may be used to prevent certain functional groups from undergoing undesired reaction. Exemplary protecting groups are described in “Protective Groups in Organic Synthesis”, 4^{th} Edition, P. G. M. Wuts; T. W. Greene, John Wiley, 2007, and references cited therein.

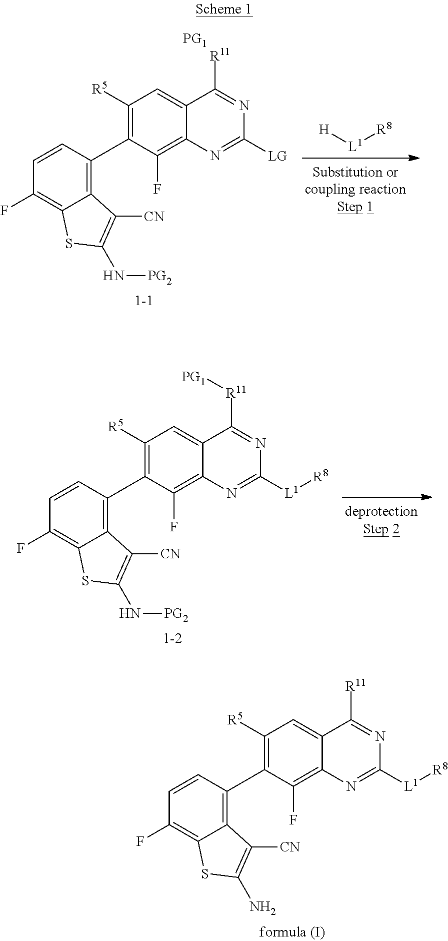

As shown in Scheme 1, in some embodiments, provided herein are methods for preparing the compounds defined as formula (I). LG (leaving group) substituted compound 1-1 (LG is Cl, F, methyl sulfonyl, or methyl sulfinyl) is converted into compound 1-2 under substitution conditions (e.g., NaH, THF) or Buchwald coupling conditions (e.g., RuPhos Pd catalyst, Cs₂CO₃, 1,4-dioxane); compound 1-2 is then deprotected (e.g., TFA and DCM to deprotect Boc group when PG₁ and PG₂ are Boc) to yield the compound defined as formula (I).

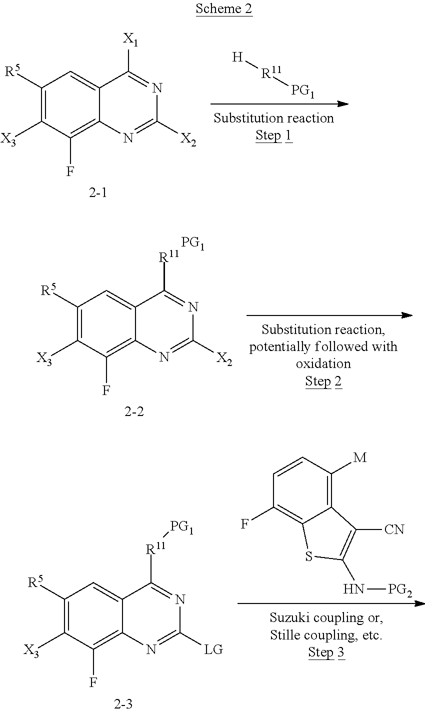

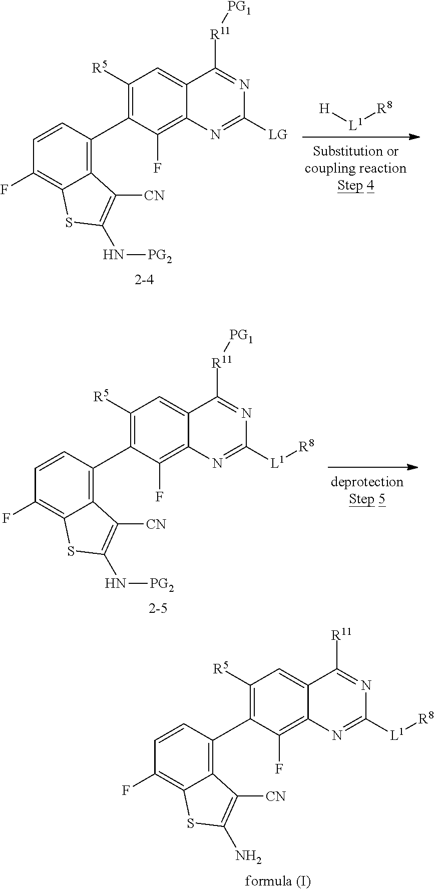

As shown in Scheme 2, in some embodiments, provided herein are methods for preparing the compounds defined as formula (I). Halogen substituted compound 2-1 (X₁, X₂, X₃ are halogen) is converted into compound 2-2 under basic conditions (e.g., NaH, THF); then compound 2-2 is converted to compound 2-3 under substitution conditions (KF, DMSO if LG is F; or MeSNa, MeOH, followed with m-CPBA oxidation if LG is methyl sulfonyl or methyl sulfinyl); compound 2-3 further undergoes metal catalyzed cross-coupling reaction such as Suzuki, Negishi, or Stille coupling (e.g. Pd(dtbpf)Cl₂, K₃PO₄, 1,4-dioxane, water for Suzuki coupling) obtain compound 2-4, wherein M can be boronic acid, boronic ester, a metal (such as Zn), tributyltin, etc.; compound 2-4 is converted into compound 2-5 under substitution conditions (e.g., NaH, THF) or Buchwald coupling conditions (e.g., RuPhos Pd catalyst, Cs₂CO₃, 1,4-dioxane); compound 2-5 is then deprotected (e.g., TFA and DCM to deprotect Boc group when PG₁ and PG₂ are Boc) to yield the compound defined as formula (I).

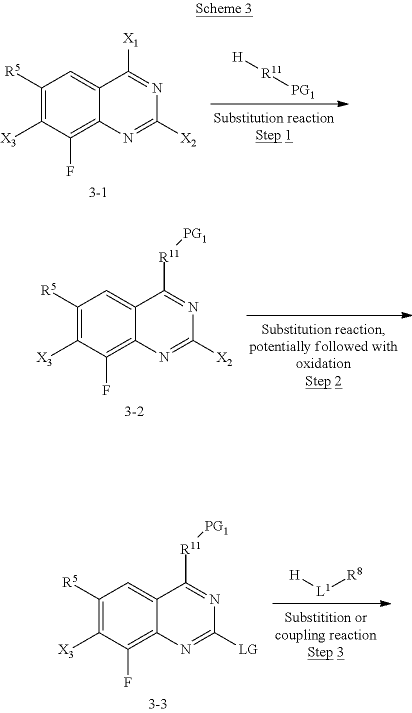

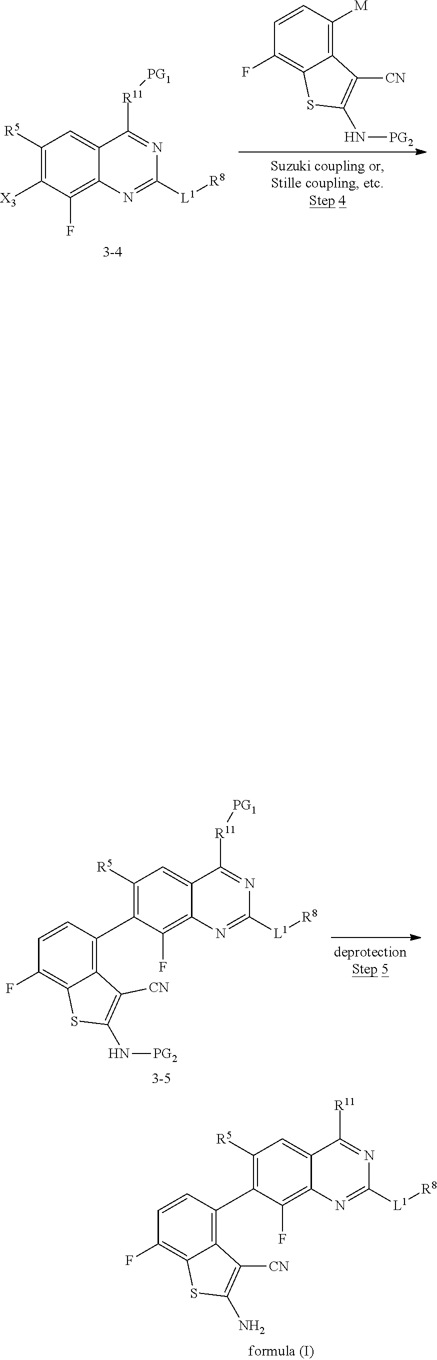

As shown in Scheme 3, in some embodiments, provided herein are methods for preparing the compounds defined as formula (I). Halogen substituted compound 3-1 (X₁, X₂, X₃ are halogen) is converted into compound 3-2 under basic conditions (e.g., NaH, THF); then compound 3-2 is converted to compound 3-3 under substitution conditions (KF, DMSO if LG is F; or MeSNa, MeOH, followed with m-CPBA oxidation if LG is methyl sulfonyl or methyl sulfinyl); compound 3-3 is converted into compound 3-4 under substitution conditions (e.g., NaH, THF) or Buchwald coupling conditions (e.g., RuPhos Pd catalyst. Cs₂CO₃, 1,4-dioxane); compound 3-4 further undergoes metal catalyzed cross-coupling reaction such as Suzuki, Negishi, or Stille coupling (e.g. Pd(dtbpf)Cl₂, K₃PO₄, 1,4-dioxane, water for Suzuki coupling) obtain compound 3-5, wherein M can be boronic acid, boronic ester, a metal (such as Zn), tributyltin, etc.; compound 3-5 is then deprotected (e.g., TFA and DCM to deprotect Hoc group when PG₁ and PG₂ are Boc) to yield the compound defined as formula (I).

The present embodiments can be understood more fully by reference to the detailed description and examples, which are intended to exemplify non-limiting embodiments.

### Examples

The examples below are intended to be purely exemplary and should not be considered to be limiting in any way. Unless otherwise specified, the experimental methods in the Examples described below are conventional methods. Unless otherwise specified, the reagents and materials are all commercially available. All solvents and chemicals employed are of analytical grade or chemical purity. Solvents are all redistilled before use. Anhydrous solvents are all prepared according to standard methods or reference methods. Silica gel (100-200 meshes) for column chromatography and silica gel (GF254) for thin-layer chromatography (TLC) are commercially available from Tsingdao Haiyang Chemical Co., Ltd. or Yantai Chemical Co., Ltd. of China; all were eluted with petroleum ether (60-90° C.)/ethyl acetate (v/v), and visualized by iodine or the solution of molybdphosphoric acid in ethanol unless otherwise specified. All extraction solvents, unless otherwise specified, were dried over anhydrous Na₂SO₄.

Unless otherwise indicated, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

Unless otherwise indicated, column chromatography purification was conducted on a Biotage system (Manufacturer: Dyax Corporation) having a silica gel column or on a silica SepPak cartridge (Waters), or was conducted on a Teledyne Isco Combiflash purification system using prepacked silica gel cartridges.

¹H NMR spectra were recorded on a Varian instrument operating at 400 MHz or 500 MHz with TMS (tetramethylsilane) as the internal standard. ¹H-NMR spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDCl₃: 7.25 ppm; CD₃OD: 3.31 ppm; D₂O: 4.79 ppm; d₆-DMSO: 2.50 ppm; d₆-acetone: 2.05; (CD₃)₂CO: 2.05) as the reference standard. When peak multiplicities are reported, the following abbreviations are used: s (singlet), d (doublet), t (triplet), q (quartet), qn (quintuplet), sx (sextuplet), m (multiplet), hr (broadened), dd (doublet of doublets), dt (doublet of triplets). Coupling constants, when given, are reported in Hertz (Hz).

LC/MS data was recorded by using Agilent1100,1200 High Performance Liquid Chromatography-Ion Trap Mass Spectrometer (LC-MSD Trap) equipped with a diode array detector (DAD) detected at 214 nm and 254 run, and an ion trap (ESI source). All compound names except the reagents were generated by ChemDraw® 19.1.

In the following examples, the following abbreviations are used:
AcOH Acetic acid
Aq. Aqueous
BINAP 2,2′-bis(diphenylphosphino)-1,1′-binaphthalene
Brine Saturated aqueous sodium chloride solution
Bn Benzyl
BnBr Benzyl Bromide
Boc Tert-butoxycarbonyl
BOP Benzotriazol-1-yl-oxy-tris-(dimethylamino)phosphonium hexafluorophosphate
CH₂Cl₂ or DCM Dichloromethane
CAN Cerium(IV) ammonium nitrate (cericammonium nitrate)
Cs₂CO₃ Cesium carbonate
DAST Diethylaminosulfur trifluoride
DCM Dichloromethane
DMF N,N-Dimethylformamide
Dppf 1,1′-bis(diphenylphosphino)ferrocene
DBU 1,8-diazabicyclo[5.4.0]undec-7-ene
DHP 3,4-Dihydro-2H-pyran
DEA or DIPEA N,N-diisopropylethylamine
DMAP 4-N,N-dimethylaminopyridine
DMB (2,4-dimethoxyphenyl)methanamine
Dess-Martin/DMP Dess-Martin Periodinane
DMF N,N-dimethylformamide
DMF-DMA N,N-Dimethylformamide dimethyl acetal purum
DMSO Dimethyl sulfoxide
DMEDA Dimethyl Ethylene Diamine
EDCI 1-Ethyl-3-(3-dimethylaminopropyl)carbodiimide hydrochloride
EtOAc or EA Ethyl acetate
EtOH Ethanol
Et₃SiH Triethyl silhydride
Et₂O or ether Diethyl ether
g Grams
h or hr Hour
HATU O-(7-Azabenzotriazol-1-yl)-N,N,N,N-tetramethyluronium hexafluorophosphate
Hex Hexane
HCl Hydrochloric acid
HMDS Hexamethyldisilazane
HOBT 1-Hydroxybenzotriazole
HPLC High-performance liquid chromatography
IBX 2-Iodylbenzoic acid
i-PrOH Isopropyl alcohol
LCMS Liquid chromatography-mass spectrometry
LDA Lithium diisopropylamide
LiHMDS Lithium Bis(trimethylsilyl)amide
K₂OsO₄•H2O Potassium osmate(VI) dihydrate
K₃PO₄ Tripotassium phosphate
mg Milligrams
mL Milliliters
mmol Millimole
MeCN Acetonitrile
MeOH Methanol
Min Minutes
ms or MS Mass spectrum
m-CPBA 2-chloranylbenzenecarboperoxoic acid
MPLC Medium Pressure Liquid Chromatography
Na₂SO₄ Sodium sulfate
NaBH(OAc)₃/STAB Sodium triacetyl borohydride
NaH Sodium hydride
NaHMDS Sodium bis(trimethylsilyl)amide
NBS N-Bromosuccinimide
NCS N-Chlorosuccinimide
NMO 4-Methylmorpholine N-oxide
NMP N-Methyl Pyrrolidone
PE petroleum ether
PMB (4-methoxyphenyl)methanamine
POCl₃ phosphorous oxychloride
PyBOP Benzotriazol-1-yl-oxytripyrrolidinophosphonium hexafluorophosphate
PddppfCl₂ [1,1′-Bis(diphenylphosphino)ferrocene]dichloropalladium(II)
Pd(dtbpf)Cl₂ [1,1′-Bis(di-tert-butylphosphino)ferrocene]dichloropalladium(II)
Pd₂(dba)₃ Tris(dibenzylideneacetone)dipalladium Prep Preparative
PTSA 4-Methylbenzenesulfonic acid
Rt or rt Room temperature
RuPhos 2-Dicyclohexylphosphino-2′,6′-diisopropoxybiphenyl
sat. Saturated
SEMCl (2-(Chloromethoxy)ethyl)trimethylsilane
TBSCl tert-Butyldimethylsilyl chloride
TEA/Et3N triethylamine
t-BuOK Potassium tert-butoxide
t-BuONa Sodium tert-butoxide
T₃P n-Propylphosphonic cyclic anhydride
TMSCN Trimethylsilyl cyanide
TFA Trifluoroacetic acid
TFAA Trifluoroacetic anhydride
THE Tetrahydrofuran
TLC thin layer chromatography
tBuXPhospd-G3 Methanesulfonato(2-di-t-butylphosphino-2′,4′,6′-tri-1-propyl-1,1′-biphenyl)(2′-amino-1,1′-biphenyl-2-yl)palladium(II)
tBuXPhos 2-Di-tert-butylphosphino-2′,4′,6′-triisopropylbiphenyl
UHP Urea hydrogen peroxide
μL Microliters
XantPhos 4,5-Bis(diphenylphosphino)-9,9-dimethylxanthene
XPhos 2-Dicyclohexylphosphino-2′,4′,6′-triisopropylbiphenyl
4CzIPN (4r,6r)-2,4,5,6-tetra(9H-carbazol-9-yl)isophthalonitrile
   Compound synthesis

### Example 1: 4-(4-(3,8-diazabicyclo[3.2.1]octan-3-yl)-2-((1-(difluoromethyl)-2-oxabicyclo[2.1.1]hexan-4-yl)methoxy)-8-fluoro-6-(trifluoromethyl)quinazolin-7-yl)-2-amino-7-fluorobenzo[b]thiophene-3-carbonitrile

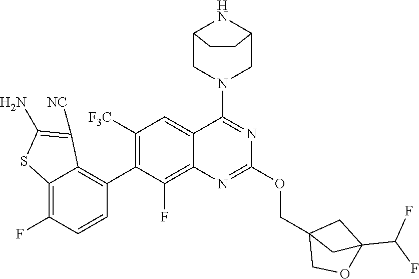 [SMILES: N#Cc1c(N)sc2c(F)ccc(-c3c(C(F)(F)F)cc4c(N5CC6CCC(C5)N6)nc(OCC56COC(C(F)F)(C5)C6)nc4c3F)c12]

### Step 1: 1-(iodomethyl)-4-(((4-methoxybenzyl)oxy)methyl)-2-oxabicyclo[2.1.1]hexane

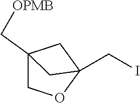

To a solution of (1-(iodomethyl)-2-oxabicyclo[2.1.1]hexan-4-yl) methanol (2.54 g, 10.0 mmol) in THE (20 mL) was added NaH (1.20 g, 30.0 mmol) at room temperature. The resulting mixture was stirred at room temperature for 1 h. Then, 1-(chloromethyl)-4-methoxybenzene was added to the reaction and the mixture was stirred at room temperature for 1 h. After completion, the reaction mixture was quenched by water (30 mL), extracted with EtOAc (100 mL), washed with saturated NaCl (50 mL×3) aqueous solution. The organic layer was dried over anhydrous Na₂SO₄, filtered and the filtrate was concentrated to give the residue. The residue was purified by silica gel column chromatography and eluted with 0-30% ethyl acetate in petroleum ether to give the title product (1.87 g, 5.0 mmol).

Step 2: (4-(((4-methoxylbenzyl)oxy)methyl)-2-oxabicyclo[2.1.1]hexan-1-yl)methanol

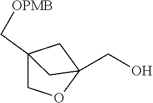

To a solution of 1-(iodomethyl)-4-(((4-methoxybenzyl)oxy)methyl)-2-oxabicyclo[2.1.1]hexane (1.87 g, 5.0 mmol) in DMSO (30 mL) was added KOAc (2.45 g, 25 mmol) at room temperature. The resulting mixture was stirred at 110° C. for 16 h. After completion, the reaction mixture was diluted with EtOAc (200 mL), washed with saturated NaCl (50 mL×3) aqueous solution. The organic layer was dried over anhydrous Na₂SO₄, filtered and the filtrate was concentrated to give the residue. The residue was added sodium ethanolate (680 mg, 10 mmol) and EtOH (10 mL). Then, the reaction mixture was stinted for 1 h at room temperature. After completion, the reaction mixture was directly concentrated to give the residue. The residue was purified by silica gel column chromatography and eluted with 0-80% ethyl acetate in petroleum ether to give the title product (528.2 mg, 2.0 mmol).

Step 3: 4-(((4-methoxybenzyl)oxy meth 1)-2-oxabicyclo[2.1.1]hexane-1-carbaldehyde

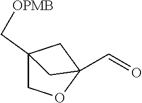

To a solution of (4-(((4-methoxybenzyl)oxy)methyl)-2-oxabicyclo[2.1.1]hexan-1-yl)methanol (528.2 mg, 2.0 mmol) in DCM (10 mL) was added DMP (848 mg, 2.0 mmol) at room temperature. The resulting mixture was stirred at room temperature for 1 h. After completion, the reaction mixture was diluted with EtOAc (100 mL) and washed with saturated NaCl (50 mL×3) aqueous solution. The organic layer was dried over anhydrous Na₂SO₄, filtered and the filtrate was concentrated to give the crude title product (1.0 g).

Step 4: 1-(difluoromethyl)-4-(((4-methoxybenzyl)oxy)methyl)-2-oxabicyclo[2.1.1]hexane

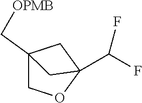

To a solution of 4-(((4-methoxybenzyl)oxy)methyl)-2-oxabicyclo[2.1.1]hexane-1-carbaldehyde (1.0 g crude) in DCM (10 mL) was added DAST (483.6 mg, 3.0 mmol) at room temperature. The resulting mixture was stirred at room temperature for 16 h. After completion, the reaction mixture was directly concentrated to give the residue. The residue was purified by silica gel column chromatography and eluted with 0-20% ethyl acetate in petroleum ether to give the title product (284.1 mg, 1.0 mmol).

### Step 5: (1-(difluoromethyl)-2-oxabicyclo[2.1.1]hexan-4-yl)methanol

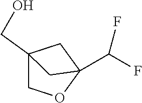 [SMILES: OCC12COC(C(F)F)(C1)C2]

To a solution of 1-(difluoromethyl)-4-(((4-methoxybenzyl)oxy)methyl)-2-oxabicyclo[2.1.1]hexane (284.1 mg, 1.0 mmol) in DCM (10 mL) and water (1 mL) was added DDQ (454.0 mg, 2.0 mmol) at room temperature. The resulting mixture was stirred at room temperature for 2 h. After completion, the reaction mixture was diluted with water (30 mL), extracted with DCM (100 mL) and washed with saturated NaCl (20 mL×3) aqueous solution. The organic layer was dried over anhydrous Na₂SO₄, filtered and the filtrate was concentrated to give the residue. The residue was purified by silica gel column chromatography and eluted with 0-60% ethyl acetate in petroleum ether to give the title product (60.0 mg, 0.36 mmol).

### Step 6: tert-butyl 3-(7-(2-((tert-butoxycarbonyl)amino)-3-cyano-7-fluorobenzo [b]thiophen-4-yl)-2-((1-(difluoromethyl)-2-oxabicyclo[2.1.1]hexan-4-yl)methoxy)-8-fluoro-6-(trifluormethyl)quinazolin-4-yl)-3,8-diazabicyclo[3.2.1]octane-8-carboxylate

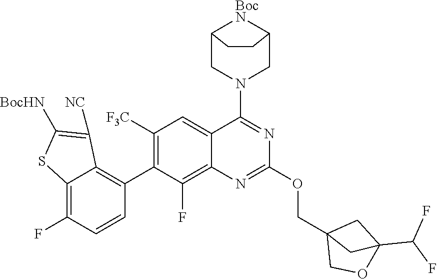 [SMILES: CC(C)(C)OC(=O)Nc1sc2c(F)ccc(-c3c(C(F)(F)F)cc4c(N5CC6CCC(C5)N6C(=O)OC(C)(C)C)nc(OCC56COC(C(F)F)(C5)C6)nc4c3F)c2c1C#N]

To a solution of (1-(difluoromethyl)-2-oxabicyclo[2.1.1]hexan-4-yl)methanol (60.0 mg, 0.36 mmol) in THE (5 mL) was added sodium hydride (26.84 mg, 0.66 mmol) at room temperature. The resulting mixture was stirred at room temperature for 1 h. Then, tert-butyl 3-(7-(2-((tert-butoxycarbonyl)amino)-3-cyano-7-fluorobenzo[b] thiophen-4-yl)-2,8-difluoro-6-(trifluoromethyl)quinazolin-4-yl)-3,8-diazabicyclo[3.2.1]octane-8-carboxylate (155 mg, 0.28 mmol) was added to the reaction mixture and stirred at room temperature for 1 h. After completion, the reaction mixture was diluted with EtOAc (50 mL) and washed with saturated NaCl (15 mL×3) aqueous solution. The organic layer was dried over anhydrous Na₂SO₄, filtered and the filtrate was concentrated to give the residue. The residue was purified by Prep-TLC (Petroleum ether:EtOAc=1:3) to give the title product (123.0 mg, 0.14 mmol). MS (ESI, m/e) [M±1] 879.3.

Step 7: 4-(4-(3,8-diazabicyclo[3.2.1]octan-3-yl)-2-((1-(difluoromethyl)-2-oxa bicyclo[2.1.1]hexan-4-yl)methoxy)-8-fluoro-6-(trifluoromethyl)quinazolin-7-yl)-2-amino-7-fluorobenzo[b]thiophene-3-carbonitrile

To a solution of tert-butyl 3-(7-(2-((tert-butoxycarbonyl)amino)-3-cyano-7-fluorobenzo[b]thiophen-4-yl)-2-((1-(difluoromethyl)-2-oxabicyclo[2.1.1]hexan-4-yl)methoxy)-8-fluoro-6-(trifluoromethyl)quinazolin-4-yl)-3,8-diazabicyclo[3.2.1] octane-8-carboxylate (123 mg, 0.14 mmol) in DCM (5 mL) was added TFA (5 mL) at room temperature. The resulting mixture was stirred at room temperature for 3 h. After completion, the reaction mixture was concentrated to give the residue. The residue was purified by HPLC to give the title product (80 mg). Then further purified by Chiral HPLC to give isomer 1 (23.0 mg) and isomer 2 (25.0 mg). Isomer 1: ¹H NMR (500 MHz, CD₃OD) δ 8.12 (s, 1H), 7.22-7.20 (m, 1H), 7.02-6.99 (m, IF), 6.12-5.90 (m, 1H), 4.77-4.74 (m, 2H), 4.56-4.45 (m, 2H), 3.87 (s, 2H), 3.71-3.63 (m, 4H), 2.06 (s, 2H), 1.83-1.75 (m, 6H); MS (ESI, m/e) [M+1]⁺ 679.4. Isomer 2: ¹H NMR (500 MHz, CD₃OD) δ 8.12 (s, IF), 7.22-7.20 (m, IF), 7.02-6.99 (m, 1H), 6.12-5.90 (m, 1H), 4.77-4.74 (m, 21-1′), 4.53-4.45 (m, 2H), 3.87 (s, 2H), 3.71-3.63 (m, 4H), 2.06 (s, 2H), 1.83-1.75 (m, 6H). MS (ESI, m/e) [M+1]⁺ 679.4.

### Example 2: 4-(2-((2-oxabicyclo[2.1.1]hexan-4-yl)methoxy)-4-(3,8-diazabicyclo[3.2.1]octan-3-yl)-6-chloro-8-fluoroquinazolin-7-yl)-2-amino-7-fluorobenzo[b]thio phene-3-carbonitrile

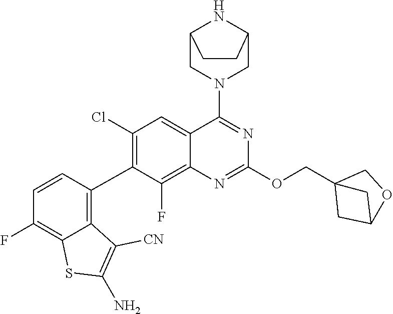 [SMILES: N#Cc1c(N)sc2c(F)ccc(-c3c(Cl)cc4c(N5CC6CCC(C5)N6)nc(OCC56COC(C5)C6)nc4c3F)c12]

### Step 1: tert-butyl 3-(7-(2-((tert-butoxycarbonyl)amino)-3-cyano-7-fluorobenzo [b]thiophen-4-yl)-6-chloro-2,8-difluoroquinazolin-4-yl)-3,8-diazabicyclo[3.2.1]octane-8-carboxylate

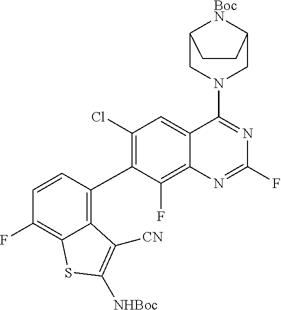 [SMILES: CC(C)(C)OC(=O)Nc1sc2c(F)ccc(-c3c(Cl)cc4c(N5CC6CCC(C5)N6C(=O)OC(C)(C)C)nc(F)nc4c3F)c2c1C#N]

The mixture of tert-butyl 3-(7-bromo-6-chloro-2,8-difluoroquinazolin-4-yl)-3,8-diazabicyclo[3.2.1]octane-8-carboxylate (244 mg, 0.5 mmol), tert-butyl (3-cyano-7-fluoro-4-(4,4,5,5-tetramethyl-1,3,2-dioxaborolan-2-yl)benzo[b]thiophen-2-yl)carbamate, DPEphosPdClz (72 mg, 0.1 mmol) and Cs₂CO₃ (490 mg, 1.5 mmol) in toluene (17.5 mL) was stirred at 95° C. for 4 h under N₂ atmosphere. After completion, the reaction mixture was purified by combi-flash (Petroleum ether/EtOAc=3:1) to give the title product (35 mg, 10% yield). MS (ESI, m/e) [M+1]⁺ 701.6.

### Step 2: tert-butyl 3-(2-((2-oxabicyclo[2.1.1]hexan-4-yl)methoxy)-7-(2-((tert-butoxycarbonyl)amino)-3-cyano-7-fluorobenzo[b]thiophen-4-yl)-6-chloro-8-fluoro quinazolin-4-yl)-3,8-diazabicyclo[3.2.1]octane-8-carboxylate

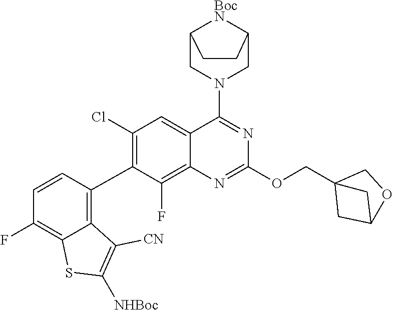 [SMILES: CC(C)(C)OC(=O)Nc1sc2c(F)ccc(-c3c(Cl)cc4c(N5CC6CCC(C5)N6C(=O)OC(C)(C)C)nc(OCC56COC(C5)C6)nc4c3F)c2c1C#N]

To a solution of (2-oxabicyclo[2.1.1]hexan-4-yl)methanol (28 mg, 0.25 mmol) in THE (5 mL) was added sodium hydride (8.0 mg, 0.334 mmol) at room temperature. The resulting mixture was stirred at room temperature for 1 h. Then, tert-butyl 3-(7-(2-((tert-butoxycarbonyl)amino)-3-cyano-7-fluorobenzo[b]thiophen-4-yl)-6-chloro-2,8-difluoroquinazolin-4-yl)-3,8-diazabicyclo[3.2.1]octane-8-carboxylate (117 mg, 0.167 mmol) was added to the reaction mixture and the mixture was stirred at room temperature for 1 h. After completion, the reaction mixture was diluted with EtOAc (50 mL) and washed with saturated NaCl (15 mL×3) aqueous solution. The organic layer was dried over anhydrous Na₂SO₄, filtered and the filtrate was concentrated to give the residue. The residue was purified by Prep-TLC (Petroleum ether:EtOAc=:1) to give the title product (100 mg, 75.8%). MS (ES, m/e) [M+1]⁺ 795.6.

### Step 3: 4-(2-((2-oxabicyclo[2.1.1]hexan-4-yl)methoxy)-4-(3,8-diazabicyclo [3.2.1]octan-3-yl)-6-chloro-8-fluoroquinazolin-7-yl)-2-amino-7-fluorobenzo[b] thiophene-3-carbonitrile

To a solution of tert-butyl 3-(2-((2-oxabicyclo[2.1.1]hexan-4-yl)methoxy)-7-(2-((tert-butoxycarbonyl)amino)-3-cyano-7-fluorobenzo[b]thiophen-4-yl)-6-chloro-8-fluoroquinazolin-4-yl)-3,8-diazabicyclo[3.2.1]octane-8-carboxylate (100 mg, 0.126 mmol) in DCM (5 mL) was added TFA (5 mL) at room temperature. The resulting mixture was stirred at room temperature for 3 h. After completion, the reaction mixture was concentrated to give the residue. The residue was purified by prep-HPLC to give the title product (60 mg) which was separated by chiral-HPLC (Prep-HPLC Equipment: Prep-HPLC-Gilson; Column: i-Cellulose-5, 21.2 mm×250 mm. 5 μm; Mobile Phase A: n-Hexane, Mobile Phase B: EtOH (0.2% 2M NH₃ in MeOH); Flow Rate: 20 mL/min; Temperature: 25° C.; Gradient: 40% B in 12 min; Retention time—isomer 1: 7.5 min; Retention time—isomer 2: 9.5 min) to give two isomers. Isomer 1 (20.47 mg, 27.3%): ¹H NMR (500 MHz, CD₃OD) δ 7.86 (s, 1H), 7.21-7.18 (m, 1H), 7.04-7.01 (m, 1H), 4.76-4.71 (m, 2H), 4.56-4.42 (m, 3H), 3.73 (s, 2H), 3.66-3.59 (m, 4H), 1.96-1.95 (m, 2H), 1.89-1.78 (m, 4H), 1.64-1.61 (m, 2H); MS (ESI, m/e) [M+1]¹ 595.4. Isomer 2 (18.65 mg, 24.9%): ¹H NMR (500 MHz, CD₃OD) δ 7.86 (s, 1H), 7.21-7.18 (m, 1H), 7.04-7.01 (m, 1H), 4.76-4.71 (m, 2H), 4.56-4.41 (m, 3H), 3.73 (s, 2H), 3.66-3.58 (m, 4H), 1.96-1.95 (m, 2H), 1.89-1.78 (m, 4H), 1.64-1.61 (m, 2H); MS (ESI, m/e) [M+1]+595.4.

### Example 3: 4-(4-(3,8-diazabicyclo[3.2.1]octan-3-yl)-8-fluoro-2-((3-methoxytetra hydrofuran-3-yl)methoxy)-6-(trifluoromethyl)quinazolin-7-yl)-2-amino-7-fluoro benzo[b]thiophene-3-carbonitrile

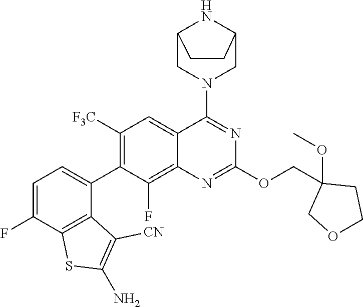 [SMILES: COC1(COc2nc(N3CC4CCC(C3)N4)c3cc(C(F)(F)F)c(-c4ccc(F)c5sc(N)c(C#N)c45)c(F)c3n2)CCOC1]

### Step 1: tert-butyl 3-(7-(2-((tert-butoxycarbonyl)amino)-3-cyano-7-fluorobenzo [b]thiophen-4-yl)-8-fluoro-2-((3-methoxytetrahydrofuran-3-yl)methoxy)-6-(trifluoro methyl)quinazolin-4-yl)-3,8-diazabicyclo[3.2.1]octane-8-carboxylate

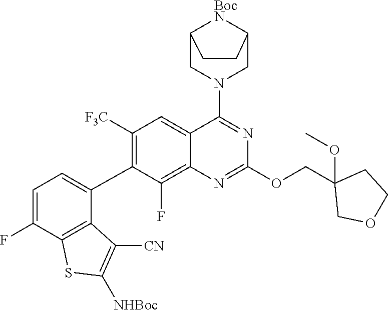 [SMILES: COC1(COc2nc(N3CC4CCC(C3)N4C(=O)OC(C)(C)C)c3cc(C(F)(F)F)c(-c4ccc(F)c5sc(NC(=O)OC(C)(C)C)c(C#N)c45)c(F)c3n2)CCOC1]

A solution of (3-methoxytetrahydrofuran-3-yl)methanol (120 mg, 0.91 mmol) in THE (20 mL) was cooled to 0° C. Then, NaH (36 mg, 0.91 mmol) was added one portion and the mixture was stirred at 0° C. for 30 min. Next, to the mixture was added tert-butyl 3-(7-(2-((tert-butoxycarbonyl)amino)-3-cyano-7-fluorobenzo[b] thiophen-4-yl)-2,8-difluoro-6-(trifluoromethyl)quinazolin-4-yl)-3,8-diazabicyclo[3.2.1]octane-8-carboxylate (240 mg, 0.32 mmol). The resulting mixture was stirred at 0° C. for 1 hour, then quenched with H₂O (10 mL), extracted with EtOAc (20 mL) three times, washed with brine (20 mL), dried over Na₂SO₄ and filtered. The filtrate was concentrated and purified by column chromatography to give the title product (144 mg, 52.17%). MS (ESI, m/e) [M+H]⁺ 847.5.

### Step 2: 4-(4-(3,8-diazabicyclo[3.2.1]octan-3-yl)-8-fluoro-2-((3-methoxytetrahydrofuran-3-yl)methoxy)-6-(trifluoromethyl)quinazolin-7-yl)-2-amino-7-fluorobenzo[b]thiophene-3-carbonitrile

To a solution of tert-butyl 3-(7-(2-((tert-butoxycarbonyl)amino)-3-cyano-7-fluorobenzo [b]thiophen-4-yl)-8-fluoro-2-((3-methoxytetrahydrofuran-3-yl)methoxy)-6-(trifluoro methyl)quinazolin-4-yl)-3,8-diazabicyclo[3.2.1]octane-8-carboxylate (140 mg, 0.16 mmol) in DCM (10 mL) was added TFA (2 mL). The mixture was stirred at room temperature for 2-3 hours and the resulting mixture was concentrated to dryness. Then, the residue was diluted with DCM/MeOH=10/1 (5 mL) and subsequentially added 5 drops of 7 M NH₃ in MeOH solution. The resulting solution was concentrated to dryness and purified by column chromatography to give the title product (85 mg, 77.3%). MS (ESI, m/e) [M+H]⁺ 647.5. The product was separated by Chiral-HPLC twice (First time: Prep-HPLC Equipment: Prep-HPLC-Gilson; Column: Amylose-1, 21.2 mm×250 mm, 5 μm; Mobile Phase A: n-Hexane. Mobile Phase B: EtOH (0.2% 2M NH₃ in MeOH); Flow Rate: 20 mL/min; Temperature: 25° C.; Gradient: 60% B in 18 min; Retention time—mixture 1: 5.5 min; Retention time—mixture 2: 14.0 min; Second time (only to separate mixture 2): Prep-HPLC Equipment: Prep-HPLC-Gilson; Column: Cellulose-C. 20 mm×250 mm, 5 μm; Mobile Phase A: n-Hexane, Mobile Phase B: EtOH (0.2% 2M NH₃ in MeOH); Flow Rate: 20 mL/min; Temperature: 25° C.; Gradient: 20% B in 40 min; Retention time—isomer 3: 28 min; Retention time—isomer 4: 36 min) to give 3 isomers. Mixture 1 (mixture of two stereoisomers): ¹H NMR (500 MHz, CD₃OD) δ 8.12 (s, 1H), 7.22-7.19 (m, 1H), 7.02-6.99 (m, 1H), 4.75-4.70 (m, 1H), 4.57-4.55 (m, 2H). 4.46-4.44 (m, 1H), 3.98-3.83 (m, 4H), 3.70-3.64 (m, 4H), 3.36 (s, 3H), 2.21-2.17 (m, 1H), 2.07-2.02 (m, 1H), 1.82 (s, 4H); MS (ESI, m/e) [M+H]⁺ 647.5. Isomer 3: ¹H NMR (500 MHz, CD₃OD) δ 8.12 (s, 1H), 7.22-7.19 (m, 1H), 7.02-6.99 (m, 1H), 4.75-4.45 (m, 4H), 3.96-3.64 (m, 8H), 3.36 (s, 3H). 2.21-2.17 (m, 1H), 2.07-2.02 (m, 1H), 1.83 (s, 4H); MS (ESI, m/e) [M+H]⁺ 647.5. Isomer 4: ¹H NMR (500 MHz, CD₃OD) δ 8.12 (s, 1H), 7.22-7.19 (m, 1H), 7.02-6.99 (m, 1H), 4.72-4.45 (m, 4H), 3.97-3.64 (m, 8H), 3.36 (s, 3H), 2.21-2.18 (m, 1H), 2.08-2.02 (m, 1H), 1.82 (s, 4H); MS (ESI, m/e) [M+H]⁺ 647.5.

### Example 4: 4-(4-(3,8-diazabicyclo[3.2.1]octan-3-yl)-8-fluoro-2-((4-methoxy tetrahydro-2H-pyran-4-yl)methoxy)-6-(trifluoromethyl)quinazol in-7-yl)-2-amino-7-fluorobenzo[b]thiophene-3 carbonitrile

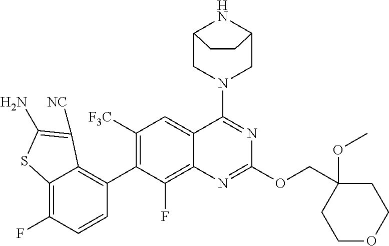 [SMILES: COC1(COc2nc(N3CC4CCC(C3)N4)c3cc(C(F)(F)F)c(-c4ccc(F)c5sc(N)c(C#N)c45)c(F)c3n2)CCOCC1]

Example 4 was prepared by similar procedure as described in Example 1 (step 6 and step 7) by replacing (1-(difluoromethyl)-2-oxabicyclo[2.1.1]hexan-4-yl)methanol with (4-methoxytetrahydro-2H-pyran-4-yl)methanol to give the title compound. It was separated by Chiral-HPLC (Prep-HPLC Equipment: Prep-HPLC-Gilson; Column: i-Cellulose-5, 21.2 mm×250 mm, 5 μm; Mobile Phase A: n-Hexane, Mobile Phase B: EtOH (0.2% 2M NH₃ in MeOH); Flow Rate: 18 mL/min; Temperature: 25+ C.; Gradient: 25% B in 15 min; Retention time—isomer 1: 10.5 min; Retention time—isomer 2: 12.5 min) to provide isomer 1 (11.2 mg) and isomer 2 (19.2 mg). Isomer 1: ¹H NMR (500 MHz, CD₃OD) δ 8.11 (s, 1H), 7.22-7.20 (m, 1H), 7.02-6.99 (m, 1H), 4.57-4.45 (m, 4H), 3.74-3.64 (m, 8H), 3.33 (s, 3H), 1.89-1.75 (m, 8H); MS (ESI, m/e) [M+H]⁺ 661.5. Isomer 2: ¹H NMR (500 MHz, CD₃OD) δ 8.11 (s, 1H), 7.22-7.20 (m, 1H), 7.02-7.01 (m, 1H), 4.50-4.45 (m, 4H), 3.74-3.64 (m, 8H), 3.33 (s, 3H), 1.89-1.75 (m, 8H); MS (ESI, m/e) [M+H]⁺ 661.4.

### Example 5: 4-(2-((2-oxabicyclo[2.1.1]hexan-4-yl)methoxy)-4-(3,8-diazabicyclo [3.2.1]octan-8-yl)-6-chloro-8-fluoroquinazolin-7-yl)-2-amino-7-fluorobenzo[b]thio phene-3-carbonitrile

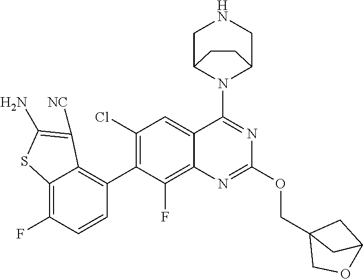 [SMILES: N#Cc1c(N)sc2c(F)ccc(-c3c(Cl)cc4c(N5C6CCC5CNC6)nc(OCC56COC(C5)C6)nc4c3F)c12]

Example 5 was prepared by similar procedure as described in Example 9 (step 1, step 2 and step 3) by replacing tert-butyl 8-(7-bromo-2,8-difluoro-6-(trifluoro methyl)quinazolin-4-yl)-3,8-diazabicyclo[3.2.1]octane-3-carboxylate with tert-butyl 8-(7-bromo-6-chloro-2,8-difluoroquinazolin-4-yl)-3,8-diazabicyclo[3.2.1]octane-3-carboxylate to give the title product. It was separated by Chiral-HPLC (Prep-HPLC Equipment: Prep-HPLC-Gilson; Column: IE, 20 mm×250 mm, 5 μm; Mobile Phase A: n-Hexane, Mobile Phase B: EtOH (0.2% 2M NH₃ in MeOH); Flow Rate: 18 mL/min; Temperature: 25° C.; Gradient: 60% B in 13 min; Retention time—isomer 1: 7.5 min; Retention time -isomer 2: 10 min) to give isomer 1 (1.03 mg) and isomer 2 (1.31 mg). Isomer 1: ¹H NMR (500 MHz, CD₃OD) δ 7.89 (s, 1H), 7.22-7.19 (m, 1H), 7.05-7.01 (m, 1H), 4.94-4.72 (m, 2H), 4.75-4.71 (m, 2H), 4.57-4.56 (m, 1H), 3.73 (s, 2H), 3.26-3.23 (m, 2H), 2.93-2.90 (m, 2H), 2.08-2.05 (m, 4H), 1.95-1.94 (m, 2H), 1.61-1.60 (m, 2H); MS (ESI, m/e) [M+H]⁺ 595.4. Isomer 2: ¹H NMR (500 MHz, CD₃OD) δ 7.89 (s, 1H). 7.22-7.19 (m, 1H), 7.05-7.01 (m, 1H). 4.94-4.72 (m, 2H), 4.75-4.71 (m, 2H), 4.57-4.56 (m, 1H), 3.73 (s, 2H), 3.25-3.23 (m, 2H), 2.92-2.90 (m, 2H), 2.07-2.05 (m, 4H), 1.95-1.94 (m, 2H), 1.61-1.60 (m, 2H); MS (ESI, m/e) [M+H]⁺ 595.3.

### Example 6: 4-(4-(3,8-diazabicyclo[3.2.1]octan-3-yl)-8-fluoro-2-((3-hydroxy tetrahydrofuran-3-yl)methoxy)-6-(trifluoromethyl)quinazolin-7-yl)-2-amino-7-fluoro benzo[b]thiophene-3-carbonitrile

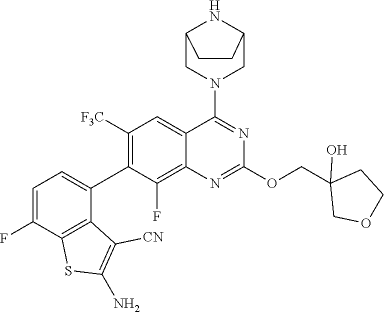 [SMILES: N#Cc1c(N)sc2c(F)ccc(-c3c(C(F)(F)F)cc4c(N5CC6CCC(C5)N6)nc(OCC5(O)CCOC5)nc4c3F)c12]

### Step 1: tert-butyl 3-(7-(2-((tert-butoxycarbonyl)amino)-3-cyano-7-fluorobenzo [b]thiophen-4-yl)-8-fluoro-2-((3-hydroxytetrahydrofuran-3-yl)methoxy)-6-(trifluoro methyl)quinazolin-4-yl)-3,8-diazabicyclo[3.2.1]octane-8-carboxylate

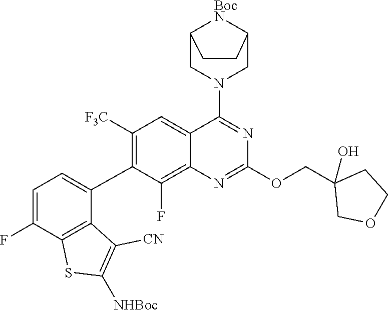 [SMILES: CC(C)(C)OC(=O)Nc1sc2c(F)ccc(-c3c(C(F)(F)F)cc4c(N5CC6CCC(C5)N6C(=O)OC(C)(C)C)nc(OCC5(O)CCOC5)nc4c3F)c2c1C#N]

To a solution of tert-butyl 3-(7-(2-((tert-butoxycarbonyl)amino)-3-cyano-7-fluoro benzo[b]thiophen-4-yl)-2,8-difluoro-6-(trifluoromethyl)quinazolin-4-yl)-3,8-diaza bicyclo[3.2.1]octane-8-carboxylate (30 mg, 0.041 mmol) and 3-(hydroxy methyl)tetra hydrofuran-3-ol (10 mg, 0.082 mmol) in THE (10 mL) was added NaH (4 mg, 0.9 mmol) at rt. The resulting mixture was stirred at rt for 2 hours. The reaction mixture was evaporated in vacuo. The residue was purified by pre-TLC (eluting with DCM/MeOH=20/1) to give the title product (12 mg, 35.2% yield). MS (ESI, m/e) [M+1]+833.6.

### Step 2: 4-(4-(3,8-diazabicyclo[3.2.1]octan-3-yl)-8-fluoro-2-((3-hydroxytetra hydrofuran-3-yl)methoxy)-6-(trifluoromethyl)quinazolin-7-yl)-2-amino-7-fluoro benzo[b]thiophene-3-carbonitrile

To a solution of tert-butyl 3-(7-(2-((tert-butoxycarbonyl)amino)-3-cyano-7-fluoro benzo[b]thiophen-4-yl)-8-fluoro-2-((3-hydroxytetrahydrofuran-3-yl)methoxy)-6-(tri fluoromethyl)quinazolin-4-yl)-3,8-diazabicyclo[3.2.1]octane-8-carboxylate (12 mg, 0.014 mmol) in DCM (2 mL) was added TFA (1 mL) at room temperature. The reaction mixture was stirred at room temperature for 1 h. The solvent was concentrated and purified by pre-HPLC to give the title product (2 mg, 27% yield, Formate). ¹H NMR (500 MHz, CD₃OD) δ 8.48 (s, 0.5H), 8.12 (s, 1H), 7.23-7.20 (m, 1H), 7.03-7.00 (m, 1H), 4.66-4.55 (m, 1H), 4.52-4.49 (m, 3H), 4.02-3.75 (m, 8H), 2.19-2.16 (m, 1H), 2.14-2.01 (m, 5H). MS (ESI, m/e) [M+1]+633.5.

### Example 7: 4-(2-((2-oxabicyclo[2.2.1]heptan-4-yl)methoxy)-4-(3,8-diazabicyclo[3.2.1]octan-3-yl)-8-fluoro-6-(trifluoromethyl)quinazolin-7-yl)-2-amino-7-fluorobenzo[b]thiophene-3-carbonitrile

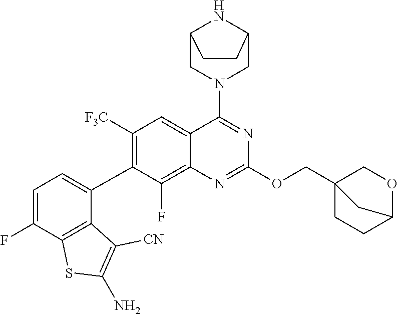 [SMILES: N#Cc1c(N)sc2c(F)ccc(-c3c(C(F)(F)F)cc4c(N5CC6CCC(C5)N6)nc(OCC56CCC(C5)OC6)nc4c3F)c12]

Example 7 was prepared by similar procedure as described in Example 3 by replacing (3-methoxytetrahydrofuran-3-yl)methanol with (5-oxabicyclo[2.1.1]hexan-1-yl)methanol to give the title product (2.09 mg). ¹H NMR (500 MHz, CD₃OD) δ 7.32 (s, 1H), 6.42 (s, 1H), 6.24-6.20 (m, 1H), 3.88-3.59 (m, 5H), 3.04-2.76 (m, 6H), 1.07-0.85 (m, 9H), 0.84-0.82 (m, 1H). MS (ESI, m/e) [M+H]⁺ 643.4.

### Example 8: methyl 3-(((7-(2-amino-3-cyano-7-fluorobenzo[h]thiophen-4-yl)-4-(3,8-diazabicyclo[3.2.1]octan-3-yl)-8-fluoro-6-(trifluoromethyl)quinazolin-2-yl) oxy)methyl)-3-methylazetidine-1-carboxylate

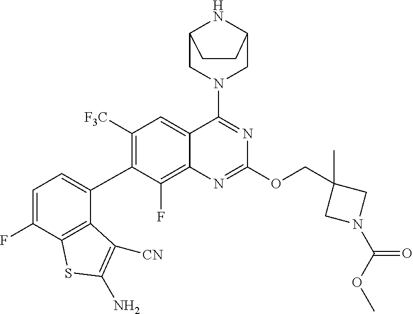 [SMILES: COC(=O)N1CC(C)(COc2nc(N3CC4CCC(C3)N4)c3cc(C(F)(F)F)c(-c4ccc(F)c5sc(N)c(C#N)c45)c(F)c3n2)C1]

### Step 1: methyl 3-(hydroxymethyl)-3-methylazetidine-1-carboxylate

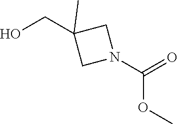

To a solution of (3-methylazetidin-3-yl)methanol hydrochloride (200 mg, 1.46 mmol) and DIPEA (565 mg, 4.38 mmol) in DCM (5 mL) was added methyl carbonochloridate (124 mg, 1.31 mmol) at 0° C. The reaction mixture was stirred at room temperature for 1 h. The solvent was added H₂O (20 mL). The organic layer was concentrated to give crude compound as colorless oil (232 mg, 100% yield). MS (ESI, m/e) [M+1]⁺160.1.

Example 8 was prepared by similar procedure as described in Example 6 by replacing 3-(hydroxy methyl)tetra hydrofuran-3-ol with methyl 3-(hydroxymethyl)-3-methylazetidine-1-carboxylate to give the title product (4 mg, 12% yield, Formate). ¹H NMR (500 MHz, CD₃OD) δ 8.48 (s, 0.5H), 8.12 (s, 1H), 7.23-7.20 (m, 1H), 7.03-7.00 (m, 1H), 4.65-4.60 (m, 1H), 4.56-4.53 (m, 1H), 4.50-4.48 (m, 2H), 4.10-3.80 (m, 4H), 3.73-3.44 (m, 7H), 2.02-1.96 (m, 4H), 1.42 (s, 3H). MS (ESI, m/e) [M+1]674.5.

### Example 9: 4-(2-((2-oxabicyclo[2.1.1]hexan-4-yl)methoxy)-4-(3,8-diaza bicyclo[3.2.1]octan-8-yl)-8-fluoro-6-(trifluoromethyl)quinazolin-7-yl)-2-amino-7-fluorobenzo[b]thiophene-3-carbonitrile

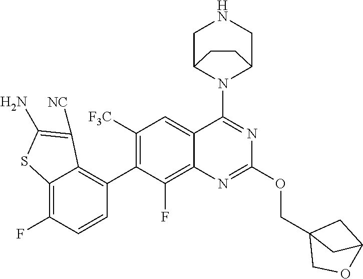 [SMILES: N#Cc1c(N)sc2c(F)ccc(-c3c(C(F)(F)F)cc4c(N5C6CCC5CNC6)nc(OCC56COC(C5)C6)nc4c3F)c12]

### Step 1: tert-butyl 8-(7-bromo-2-chloro-8-fluoro-6-(trifluoromethyl)quinazolin-4-yl)-3,8-diazabicyclo[3.2.1]octane-3-carboxylate

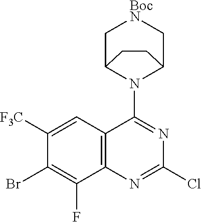 [SMILES: CC(C)(C)OC(=O)N1CC2CCC(C1)N2c1nc(Cl)nc2c(F)c(Br)c(C(F)(F)F)cc12]

To a solution of 7-bromo-2,4-dichloro-8-fluoro-6-(trifluoromethyl)quinazoline (10.0 g, 27.6 mmol) and DIPEA (7.10 g, 55.2 mmol) in DCM (200 mL) was added tert-butyl 3,8-diazabicyclo[3.2.1]octane-3-carboxylate (5.86 g, 27.6 mmol) at room temperature, The resulting mixture was stirred at room temperature for 2 hrs. After completion. The reaction mixture was diluted with water (100 mL), extracted with DCM (500 mL), and washed with saturated NaCl (100 mL×3). The organic layer was dried over anhydrous Na₂SO₄, filtered and the filtrate was concentrated to give the residue. The residue was purified by silica gel column chromatography, eluted with 0-40% ethyl acetate in petroleum ether to give the title product (10.0 g. 18.6 mmol).

### Step 2: tert-butyl 8-(7-bromo-2,8-difluoro-6-(trifluoromethyl)quinazolin-4-yl)-3,8-diazabicyclo[3.2.1]octane-3-carboxylate

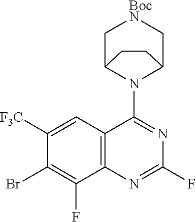 [SMILES: CC(C)(C)OC(=O)N1CC2CCC(C1)N2c1nc(F)nc2c(F)c(Br)c(C(F)(F)F)cc12]

To a solution of tert-butyl 8-(7-bromo-2-chloro-8-fluoro-6-(trifluoromethyl)quinazolin-4-yl)-3,8-diazabicyclo[3.2.1]octane-3-carboxylate (10.0 g, 18.6 mmol) in DMSO (300 mL) was added KF (5.8 g, 100 mmol) at room temperature. The resulting mixture was stirred at 100° C. for 16 hrs. After completion. The reaction mixture was diluted with EtOAc (500 mL), washed with saturated NaCl aqueous solution (100 mL×3). The organic layer was dried over anhydrous Na₂SO₄, filtered and the filtrate was concentrated to give the residue. The residue was purified by silica gel column chromatography, eluted with 0-80% ethyl acetate in petroleum ether to give the title product (6.5 g, 12.4 mmol).

### Step 3: tert-butyl 8-(2-((2-oxabicyclo[2.1.1]hexan-4-yl)methoxy)-7-bromo-8-fluoro-6-(trifluoromethyl)quinazolin-4-yl)-3,8-diazabicyclo[3.2.1]octane-3-carboxylate

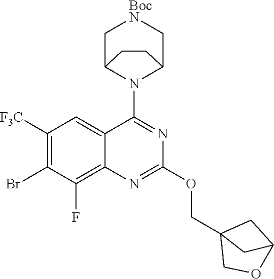 [SMILES: CC(C)(C)OC(=O)N1CC2CCC(C1)N2c1nc(OCC23COC(C2)C3)nc2c(F)c(Br)c(C(F)(F)F)cc12]

To a solution of (2-oxabicyclo[2.1.1]hexan-4-yl)methanol (114.1 mg, 1.0 mmol) in THE (5 mL) was added sodium hydride (80.0 mg, 2.0 mmol) at room temperature. The resulting mixture was stirred at room temperature for 1 h. Then, tert-butyl 8-(7-bromo-2,8-difluoro-6-(trifluoromethyl)quinazolin-4-yl)-3,8-diazabicyclo[3.2.1] octane-3-carboxylate (552 mg, 1.0 mmol) was added to the reaction mixture and stirred at room temperature for 1 h. After completion, the reaction mixture was quenched with water (10 mL), extracted with EtOAc (100 mL) and washed with saturated NaCl (15 mL×3) aqueous solution. The organic layer was dried over anhydrous Na₂SO₄, filtered and the filtrate was concentrated to give the residue. The residue was purified by silica gel column chromatography and eluted with 0-100% ethyl acetate in petroleum ether to give the title product (492.8 mg, 0.8 mmol). MS (ESI, m/e) [M+1]⁺ 617.1.

### Step 4: tert-butyl 8-(2-((2-oxabicyclo[2.1.1]hexan-4-yl)methoxy)-7-(2-((tert-butoxycarbonyl)amino)-3-cyano-7-fluorobenzo[b]thiophen-4-yl)-8-fluoro-6-(trifluoromethyl)quinazolin-4-yl)-3,8-diazabicyclo[3.2.1]octane-3-carboxylate

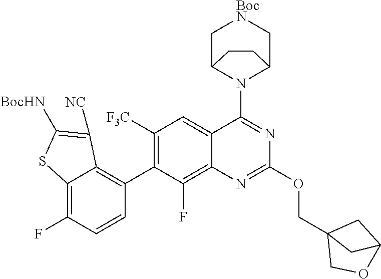

To a solution of tert-butyl 8-(2-((2-oxabicyclo[2.1.1]hexan-4-yl)methoxy)-7-bromo-8-fluoro-6-(trifluoromethyl)quinazolin-4-yl)-3,8-diazabicyclo[3.2.1]octane-3-carboxylate (308.5 mg, 0.5 mmol), tert-butyl (3-cyano-7-fluoro-4-(4,4,5,5-tetramethyl-1,3,2-dioxaborolan-2-yl)benzo[b]thiophen-2-yl)carbamate (418 mg, 1.0 mmol) and DPEPhosPdCl₂ (71.6 mg, 0.1 mmol) in toluene (15 mL) was added Cs₂CO₃ (325.8 mg. 1.0 mmol) at room temperature. The resulting mixture was stirred at 100° C. for 16 h. After completion, the reaction mixture was diluted with EtOAc (50 mL) and washed with saturated NaCl (25 mL×3) aqueous solution. The organic layer was dried over anhydrous Na₂SO₄, filtered and the filtrate was concentrated to give the residue. The residue was purified by Prep-TLC (pure EtOAc) to give the title product (162.6 mg. 0.20 mmol). MS (ESI, m/e) [M+1]⁺ 829.4.

### Step 5: 4-(2-((2-oxabicyclo[2.1.1]hexan-4-yl)methoxy)-4-(3,8-diaza bicyclo[3.2.1]octan-8-yl)-8-fluoro-6-(trifluoromethyl)quinazolin-7-yl)-2-amino-7-fluorobenzo[b]thiophene-3-carbonitrile

To a solution of tert-butyl 8-(2-((2-oxabicyclo[2.1.1]hexan-4-yl)methoxy)-7-(2-((tert-butoxycarbonyl)amino)-3-cyano-7-fluorobenzo[b]thiophen-4-yl)-8-fluoro-6-(trifluoromethyl)quinazolin-4-yl)-3,8-diazabicyclo[3.2.1]octane-3-carboxylate (162.6 mg, 0.20 mmol) in DCM (5 mL) was added TFA (5 mL) at room temperature. The resulting mixture was stirred at room temperature for 3 h. After completion, the reaction mixture was concentrated to give the residue. The residue was purified by HPLC to give the title product (80 mg) which was furtherly purified by Chiral-HPLC (Prep-HPLC Equipment: Prep-HPLC-Gilson; Column: IE, 20 mm×250 mm, 5 μm; Mobile Phase A: n-Hexane, Mobile Phase B: EtOH (0.2% 2M NH₃ in MeOH); Flow Rate: 18 mL/min; Temperature: 25° C.; Gradient: 40% B in 16 min; Retention time—isomer 1: 10 min; Retention time—isomer 2: 14.5 min) to give isomer 1 (40.5 mg) and isomer 2 (19.0 mg). Isomer 1: ¹H NMR (500 MHz, CD₃OD) δ 8.14 (s, 1H), 7.23-7.20 (m, 1H), 7.02-6.99 (m, 1H), 4.97-4.75 (m, 2H), 4.76-4.75 (m, 2H), 4.57-4.56 (m, 1H), 3.73 (s, 2H), 3.25-3.23 (m, 2H), 2.95-2.92 (m, 2H), 2.09-2.08 (m, 4H), 1.96-1.95 (m, 2H), 1.61-1.60 (m, 2H); MS (EST, m/e) [M+1]⁺ 629.4. Isomer 2: ¹H NMR (500 MHz, CD₃OD) δ 8.14 (s, 1H), 7.23-7.20 (m, 1H), 7.02-6.99 (m, 1H), 4.97-4.75 (m, 2H), 4.76-4.75 (m, 2H), 4.57-4.56 (m, 1H), 3.73 (s, 2H), 3.25-3.23 (m, 2H), 2.95-2.92 (m, 2H), 2.09-2.08 (m, 4H), 1.96-1.95 (m, 2H), 1.61-1.60 (m, 2H); MS (EST, m/e) [M+1] 629.3.

### Example 10: 3-(((7-(2-amino-3-cyano-7-fluorobenzo[b]thiophen-4-yl)-4-(3,8-diazabicyclo[3.2.1]octan-3-yl)-8-fluoro-6-(trifluoromethyl)quinazolin-2-yl)oxy) methyl)tetrahydrofuran-3-carbonitrile

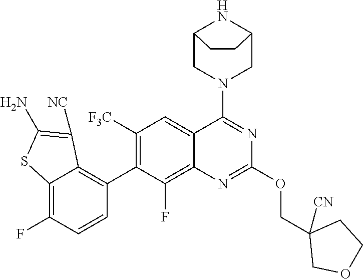 [SMILES: N#Cc1c(N)sc2c(F)ccc(-c3c(C(F)(F)F)cc4c(N5CC6CCC(C5)N6)nc(OCC5(C#N)CCOC5)nc4c3F)c12]

Example 10 was prepared by similar procedure as described in Example 1 (step 6 and step 7) by replacing (1-(difluoromethyl)-2-oxabicyclo[2.1.1]hexan-4-yl) methanol with 3-(hydroxymethyl)tetrahydrofuran-3-carbonitrile to give the title compound. It was separated by Chiral-HPLC (Prep-HPLC Equipment: Prep-HPLC-Gilson; Column: IE, 20 mm×250 mm, 5 μm; Mobile Phase A: n-Hexane. Mobile Phase B: EtOH (0.2% 2M NH₃ in MeOH); Flow Rate: 18 mL/min; Temperature: 25° C.; Gradient: 40% B in 18 min; Retention time—isomer 1: 9 min; Retention time -isomer 2: 11 min; Retention time—isomer 3: 13 min; Retention time—isomer 4: 17 min) to provide isomer 1 (30.5 mg), isomer 2 (27.2 mg), isomer 3 (26.5 mg) and isomer 4 (23.2 mg). Isomer 1: ¹H NMR (500 MHz, CD₃OD) δ 8.14 (s, 1H), 7.22-7.20 (m, 1H), 7.02-6.99 (m, 1H)—, 4.65-4.48 (m, 4H), 4.10-4.01 (m, 4H), 3.72-3.66 (m, 4H), 2.48-2.25 (m, 2H), 1.95-1.69 (m, 4H); MS (EST, m/e) [M+H]⁺ 642.3. Isomer 2: ¹H NMR (500 MHz, CD₃OD) δ 8.14 (s, 1H), 7.22-7.20 (m, 1H), 7.02-7.01 (m, 1H), 4.67-4.48 (m, 4H), 4.10-3.94 (m, 4H), 3.72-3.67 (m, 4H), 2.53-2.43 (m, 1H), 2.36-2.26 (m, 1H), 1.93-1.74 (m, 4H); MS (ESI, m/e) [M+H]⁺ 642.3. Isomer 3: ¹H NMR (500 MHz, CD₃OD) δ 8.14 (s, 1H), 7.22-7.20 (m, 1H), 7.02-6.99 (m, 1H), 4.67-4.49 (m, 4H), 4.10-3.94 (m, 4H), 3.72-3.59 (m, 4H), 2.51-2.43 (m, 1H), 2.37-2.24 (m, 1H), 1.93-1.74 (m, 4H); MS (ESI, m/e) [M+H]⁺ 642.3. Isomer 4: ¹H NMR (500 MHz, CD₃OD) δ 8.14 (s, 1H), 7.22-7.20 (m, 1H), 7.02-6.99 (m, 1H), 4.65-4.48 (m, 4H), 4.10-4.01 (m, 4H), 3.72-3.55 (m, 4H), 2.53-2.39 (m, 1H), 2.39-2.24 (m, 1H), 1.90-1.74 (m, 4H); MS (ESI, m/e) [M+H]⁺ 642.3.

### Example 11: 4-(4-(3,8-diazabicyclo[3.2.1]octan-3-yl)-8-fluoro-2-((1-(methoxy methyl)-2-oxabicyclo[2.1.1]hexan-4-yl)methoxy)-6-(trifluoromethyl)quinazolin-7-yl)-2-amino-7-fluorobenzo[b]thiophene-3-carbonitrile

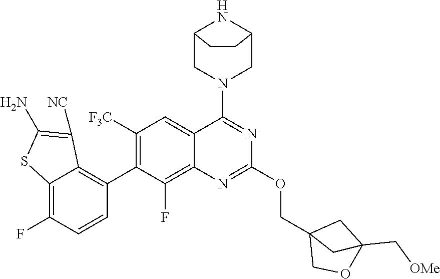 [SMILES: COCC12CC(COc3nc(N4CC5CCC(C4)N5)c4cc(C(F)(F)F)c(-c5ccc(F)c6sc(N)c(C#N)c56)c(F)c4n3)(CO1)C2]

### Step 1: (1-(methoxymethyl)-2-oxabicyclo[2.1.1]hexan-4-yl)methanol

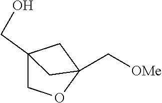 [SMILES: COCC12CC(CO)(CO1)C2]

To a solution of (1-(iodomethyl)-2-oxabicyclo[2.1.1]hexan-4-yl)methanol (2.54 g, 10.0 mmol) in MeOH (20 mL) was added sodium methanolate (5.40 g, 100.0 mmol) at room temperature. The resulting mixture was stirred at 70° C. for 16 h. After completion, the reaction mixture was diluted with water (30 mL), extracted with EtOAc (100 mL) and washed with saturated NaCl (50 mL×3) aqueous solution. The organic layer was dried over anhydrous Na₂SO₄, filtered and the filtrate was concentrated to give the crude product (500 mg).

Example 11 was prepared by similar procedure as described in Example 1 (step 6 and step 7) by replacing (I -(difluoromethyl)-2-oxabicyclo[2.1.1]hexan-4-yl) methanol with (1-(methoxymethyl)-2-oxabicyclo[2.1.1]hexan-4-yl)methanol to give the title product. It was separated by Chiral-HPLC (Prep-HPLC Equipment: Prep-HPLC-Gilson; Column: IE, 20 mm×250 mm, 5 μm; Mobile Phase A: n-Hexane, Mobile Phase B: EtOH (0.2% 2M NH₃ in MeOH); Flow Rate: 18 mL/min; Temperature: 25° C.; Gradient: 40% B in 22 min; Retention time—isomer 1: 9 min; Retention time—isomer 2: 19 min) to provide isomer 1 (1.79 mg) and isomer 2 (1.59 mg). Isomer 1: ¹H NMR (500 MHz, CD₃OD) δ 8.11 (s, 1H), 7.22-7.20 (m, 1H), 7.02-6.99 (m, 1H), 4.75-4.72 (m, 2H), 4.58-4.46 (m, 2H), 3.81 (s, 2H), 3.71-3.63 (m, 6H), 3.38 (s, 3H), 1.89-1.83 (m, 6H), 1.68-1.67 (m, 2H); MS (ESI, m/e) [M+H]⁺ 673.4. Isomer 2: ¹H NMR (500 MHz, CD₃OD) δ 8.11 (s, 1H), 7.22-7.20 (m, 1H), 7.02-6.99 (m, 1H), 4.75-4.72 (m, 2H), 4.58-4.46 (m, 2H), 3.81 (s, 2H), 3.71-3.63 (m, 6H), 3.38 (s, 3H), 1.89-1.83 (m, 6H), 1.68-1.67 (m, 2H); MS (ESI, m/e) [M+H]⁺ 673.4.

### Example 12: 4-(4-(3,8-diazabicyclo[3.2.1]octan-3-yl)-2-((1-ethyl-2-oxabicyclo [2.1.1]hexan-4-yl)methoxy)-8-fluoro-6-(trifluoromethyl)quinazolin-7-yl)-2-amino-7-fluorobenzo[h]thiophene-3-carbonitrile

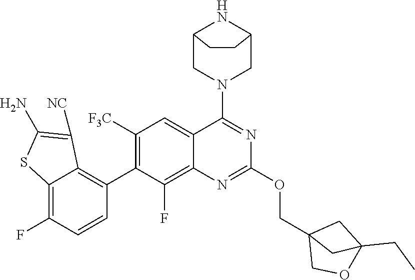 [SMILES: CCC12CC(COc3nc(N4CC5CCC(C4)N5)c4cc(C(F)(F)F)c(-c5ccc(F)c6sc(N)c(C#N)c56)c(F)c4n3)(CO1)C2]

Example 12 was prepared by similar procedure as described in Example 1 (step 6 and step 7) by replacing (1-(difluoromethyl)-2-oxabicyclo[2.1.1]hexan-4-yl) methanol with (1-ethyl-2-oxabicyclo[2.1.1]hexan-4-yl)methanol to give the title product (0.7 mg). ¹H NMR (500 MHz, CD₃OD) δ 8.12 (s, 1H), 7.21-7.20 (m, 1H), 7.04-7.00 (m, 1H), 4.80-4.49 (m, 4H), 4.18-4.01 (m, 2H), 3.93-3.76 (m, 3H), 3.70-3.64 (m, 1H), 2.18-1.97 (m, 5H), 1.96-1.87 (m, 1H), 1.85-1.72 (m, 4H), 1.61-1.55 (m, 3H). MS (ESI, m/e) [M+H]⁺ 657.5.

### Example 13: 4-(2-((1-acetyl-3-methylazetidin-3-yl)methoxy)-4-(3,8-diaza bicyclo[3.2.1]octan-3-yl)-8-fluoro-6-(trifluoromethyl)quinazolin-7-yl)-2-amino-7-fluorobenzo[b]thiophene-3-carbonitrile

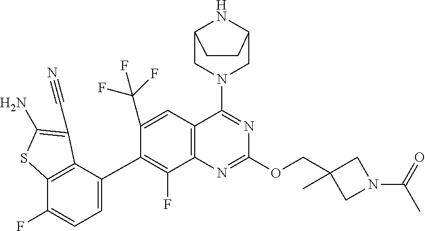 [SMILES: CC(=O)N1CC(C)(COc2nc(N3CC4CCC(C3)N4)c3cc(C(F)(F)F)c(-c4ccc(F)c5sc(N)c(C#N)c45)c(F)c3n2)C1]

### Step 1: 1-(3-(hydroxymethyl)-3-methylazetidin-1-yl)ethan-1-one

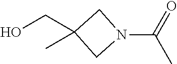

To a 25 mL round bottom flask was added (3-methylazetidin-3-yl)methanol hydrochloride (530 mg, 4.0 mmol), DCM (20 mL), pyridine (530 mg, 6.7 mmol) and acetyl chloride (220 mg, 2.8 mmol). The reaction was stirred at rt for 3 h. The reaction mixture was concentrated under vacuum. The residue was purified by flash (MeOH:DCM=15-20%) to give the title product (300 mg, 52.1% yield). MS (EST, m/e) [M+H]⁺ 144.3.

### Step 2: tert-butyl 3-(2-((1-acetyl-3-methylazetidin-3-yl)methoxy)-7-(2-((tert-butoxycarbonyl)amino)-3-cyano-7-fluorobenzo[b]thiophen-4-yl)-8-fluoro-6-(trifluoromethyl)quinazolin-4-yl)-3,8-diazabicyclo[3.2.1]octane-8-carboxylate

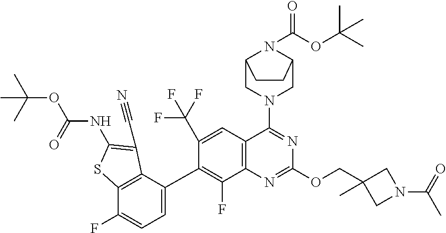 [SMILES: CC(=O)N1CC(C)(COc2nc(N3CC4CCC(C3)N4C(=O)OC(C)(C)C)c3cc(C(F)(F)F)c(-c4ccc(F)c5sc(NC(=O)OC(C)(C)C)c(C#N)c45)c(F)c3n2)C1]

To a 20 mL sealed tube was added 1-(3-(hydroxymethyl)-3-methylazetidin-1-yl)ethan-1-one (40 mg, 0.055 mmol), tert-butyl 3-(7-(2-((tert-butoxycarbonyl)amino)-3-cyano-7-fluorobenzo[b]thiophen-4-yl)-2,8-difluoro-6-(trifluoromethyl) quinazolin-4-yl)-3,8-diazabicyclo[3.2.1]octane-8-carboxylate (40 mg, 0.278 mmol), THE (3 mL) and NaH (5 mg, 0.125 mmol. 60%). The mixture was stirred at rt for 16 h. The reaction was quenched by saturated NH₄Cl (10 mL) aqueous solution and extracted by EtOAc (20 mL×3). The combined organic phase was dried by anhydrous Na₂SO₄ and concentrated under vacuum. The residue was purified by flash (MeOH:DCM=5˜10%) to give the title product (36 mg, 76.8% yield). MS (ESI, m/e) [M+H]⁺ 858.3.

### Step 3: 4-(2-((1-acetyl-3-methylazetidin-3-yl)methoxy)-4-(3,8-diazabicyclo [3.2.1]octan-3-yl)-8-fluoro-6-(trifluoromethyl)quinazolin-7-yl)-2-amino-7-fluoro benzo[b]thiophene-3-carbonitrile

To a 25 mL round bottom flask was added tert-butyl 3-(2((1-acetyl-3-methylazetidin-3-yl)methoxy)-7-(2-((tert-butoxycarbonyl)amino)-3-cyano-7-fluorobenzo[b]thiophen-4-yl)-8-fluoro-6-(trifluoromethyl)quinazolin-4-yl)-3,8-diazabicyclo[3.2.1]octane-8-carboxylate (36 mg, 0.042 mmol) and TFA (4 mL). The reaction was stinted at rt for 16 h. The reaction was concentrated under vacuum. The residue was dissolved in EtOAc (20 mL) and saturated Na₂CO₃ aqueous solution (10 mL) was added. The solution was stirred at rt for 10 min and extracted by EtOAc (20 mL×2). The combined organic phase was washed with brine (20 mL), dried over Na₂SO₄ and concentrated under vacuum. The residue was purified by Prep-HPLC (Column: XBridge Shield RP18 OBD Column; Mobile Phase A: Water (10 mmol/L NH₄HCO₃+0.1% NH₃•H₂O), Mobile Phase B: ACN; Gradient: 25% B to 65% B in 9 min) to give the title product (4.2 mg, 15.2% yield). ¹H NMR (500 MHz, CD₃OD) δ 8.12 (s, 1H), 7.23-7.20 (m, 1H), 7.03-7.00 (m, 1H), 4.69-4.44 (m, 4H), 4.27-4.16 (m, 1H), 4.10-3.75 (m, 6H), 3.69-3.68 (m, 1H). 2.03 (s, 4H), 1.88 (s, 3H), 1.45 (s, 3H). MS (ESI, m/e) [M+H]⁺ 658.3.

### Example 14: 4-(4-(3,8-diazabicyclo[3.2.1]octan-3-yl)-2-((3-(difluoromethyl) tetrahydrofuran-3-yl)methoxy)-8-fluoro-6-(trifluoromethyl)quinazolin-7-yl)-2-amino-7-fluorobenzo[b]thiophene-3-carbonitrile

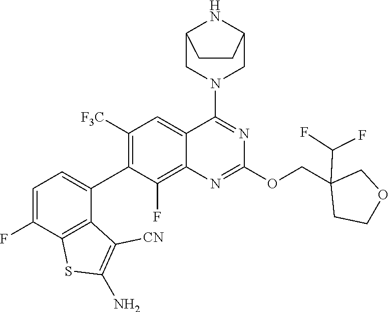 [SMILES: N#Cc1c(N)sc2c(F)ccc(-c3c(C(F)(F)F)cc4c(N5CC6CCC(C5)N6)nc(OCC5(C(F)F)CCOC5)nc4c3F)c12]

Example 14 was prepared by similar procedure as described in Example 3 by replacing (3-methoxytetrahydrofuran-3-yl)methanol with (3-(difluoromethyl) tetrahydrofuran-3-yl) methanol to give the title racemic product (84.39 mg) which was separated by Chiral-HPLC (Prep-HPLC Equipment: Prep-HPLC-Gilson; Column: IE, 20 mm×250 mm, 5 μm; Mobile Phase A: n-Hexane, Mobile Phase B: EtOH (0.2% 2M NH₃ in MeOH); Flow Rate: 18 mL/min; Temperature: 25° C.; Gradient: 30% B in 22 min; Retention time—isomer 1: 7.5 min; Retention time—isomer 2: 10 min; Retention time—isomer 3: 17 min; Retention time—isomer 4: 18 min) to obtain four isomers. Isomer 1: ¹H NMR (500 MHz, CD₃OD) δ 8.13 (s, 1H), 7.23-7.20 (m, 1H), 7.03-6.99 (m, 1H), 6.30-6.08 (m, 1H), 4.59-4.67 (m, 4H), 3.95-3.85 (m, 4H), 3.74-3.65 (m, 4H), 2.37-2.09 (m, 1H), 2.00-1.92 (m, 1H), 1.89-1.73 (m, 4H); MS (ESI, m/e) [M+H]⁺ 667.5. Isomer 2: ¹H NMR (500 MHz, CD₃OD) δ 8.13 (s, 1H), 7.23-7.20 (m, 1H), 7.03-6.99 (m, 1H), 6.30-6.08 (m, 1H), 4.69-4.40 (m, 4H), 4.01-3.81 (m, 4H), 3.74-3.65 (m, 4H), 2.22-2.17 (m, 1H), 1.97-1.93 (m, 1H), 1.91-1.73 (m, 4H); MS (ESI, m/e) [M+H]⁺ 667.4. Isomer 3: ¹H NMR (500 MHz, CD₃OD) δ 8.13 (s, 1H), 7.23-7.20 (m, 1H), 7.03-6.99 (m, 1H), 6.30-6.08 (m, 1H), 4.64-4.40 (m, 4H), 4.03-3.79 (m, 4H), 3.72-3.64 (m, 4H), 2.21-2.17 (m, 1H), 2.05-1.93 (m, 1H), 1.89-1.71 (m, 4H); MS (ESI, m/e) [M+H]⁺ 667.4. Isomer 4: ¹H NMR (500 MHz, CD₃OD) δ 8.13 (s, 1H), 7.23-7.20 (m, 1H), 7.02-6.99 (m, 1H), 6.30-6.08 (m, 1H), 4.66-4.39 (m, 4H), 4.04-3.81 (m, 4H), 3.69-3.63 (m, 4H), 2.29-2.12 (m, 1H), 2.04-1.93 (m, 1H), 1.82-1.78 (m, 4H); MS (ESI, m/e) [M+H]⁺ 667.4.

### Example 15: 4-(4-(3,8-diazabicyclo[3.2.1]octan-3-yl)-8-fluoro-2-((1-methyl-2-oxabicyclo[2.1.1]hexan-4-yl)methoxy)quinazolin-7-yl)-2-amino-7-fluorobenzo[b] thiophene-3-carbonitrile

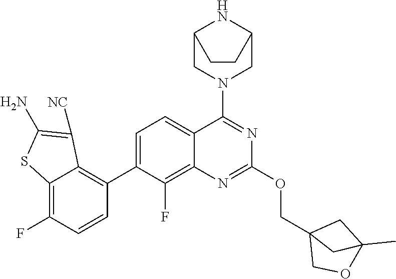 [SMILES: CC12CC(COc3nc(N4CC5CCC(C4)N5)c4ccc(-c5ccc(F)c6sc(N)c(C#N)c56)c(F)c4n3)(CO1)C2]

### Step 1: tert-butyl 3-(7-brom, diazabicyclo[3.2.1]octane-8-carboxylate

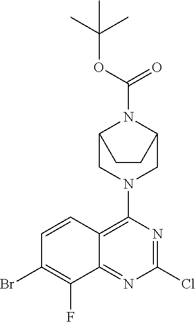 [SMILES: CC(C)(C)OC(=O)N1C2CCC1CN(c1nc(Cl)nc3c(F)c(Br)ccc13)C2]

To a solution of 7-bromo-2,4-dichloro-8-fluoroquinazoline (6.0 g, 20 mmol) and DIEA (5.16 g, 40 mmol) in DCM (100 mL) was added tert-butyl 3,8-diazabicyclo[3.2.1]octane-8-carboxylate (4.24 g, 20 mmol) at room temperature. The resulting mixture was stirred at room temperature for 2 hrs. After completion. The reaction mixture was diluted with DCM (200 mL), washed with saturated water (150 mL×3). The organic layer was dried over anhydrous Na₂SO₄, filtered and the filtrate was concentrated to give the residue. The residue was purified by silica gel (Petroleum ether:EtOAc=3:1) to give the title product (6.5 g, 69.1%). MS (ESI, m/e) [M+1]⁺ 471.4, 473.4.

### Step 2: tert-butyl 3-(7-bromo-2,8-difluoroquinazolin-4-yl)-3,8-diazabicyclo[3.2.1]octane-8-carboxylate

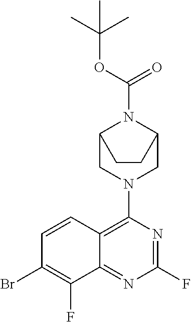 [SMILES: CC(C)(C)OC(=O)N1C2CCC1CN(c1nc(F)nc3c(F)c(Br)ccc13)C2]

The solution of tert-butyl (1R,5S)-3-(7-bromo-2-chloro-8-fluoroquinazolin-4-yl)-3,8-diazabicyclo[3.2.1]octane-8-carboxylate (4.73 g, 10 mmol) and KF (5.8 g, 100 mmol) in DMSO (40 mL) was stirred at 100C for 15 hrs. After completion. The reaction mixture was cooled to OC and decanted into ice-water, filtered to give the title product (2.5 g, 55.1%). ¹H NMR (500 MHz, DMSO) δ 7.89-7.87 (m, 1H), 7.65-7.62 (m, 1H), 4.40-4.38 (m, 2H), 4.26-4.23 (m, 2H), 3.62-3.60 (m, 2H), 1.81-1.77 (m, 2H), 1.65-1.63 (m, 2H), 1.46 (s, 9H). MS (ESI, m/e) [M+1]⁺ 455.3, 457.3

### Step 3: tert-butyl 3-(7-(2-((tert-butoxycarbonyl)amino)-3-cyano-7-fluorobenzo [b]thiophen-4-yl)-2,8-difluoroquinazolin-4-yl)-3,8-diazabicyclo[3.2.1]octane-8-carboxylate

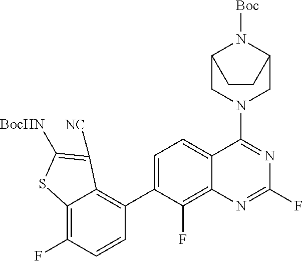 [SMILES: CC(C)(C)OC(=O)Nc1sc2c(F)ccc(-c3ccc4c(N5CC6CCC(C5)N6C(=O)OC(C)(C)C)nc(F)nc4c3F)c2c1C#N]

To a solution of tert-butyl 3-(7-bromo-2,8-difluoroquinazolin-4-yl)-3,8-diaza bicyclo[3.2.1]octane-8-carboxylate (400 mg, 0.87 mmol), tert-butyl (3-cyano-7-fluoro-4-(4,4,5,5-tetramethyl-1,3,2-dioxaborolan-2-yl)benzo[b]thiophen-2-yl)carbamate (365 mg, 0.87 mmol) and Pd(dtbpf)Cl₂ (228 mg, 0.35 mmol) in 1,4-dioxane (15 mL) and H₂O (3 mL) was added K₃PO₄ (556 mg, 2.62 mmol) at room temperature. Then, the reaction mixture was stirred at 90° C. for about 4 h. After completion, the reaction mixture was diluted with EtOAc (50 mL) and washed with saturated NaCl aqueous solution. The organic layer was dried over anhydrous Na₂SO₄ and filtered. The filtrate was concentrated to give the crude product. The residue was purified by silica gel column chromatography and eluted with 0-40% DCM in ethyl acetate to give the title product (350 mg, 0.53 mmol). MS (ESI, m/e) [M+1]⁺ 667.2.

### Step 4: tert-butyl 3-(7-(2-((tert-butoxycarbonyl)amino)-3-cyano-7-fluorobenzo [b]thiophen-4-yl)-8-fluoro-2-((1-methyl-2-oxabicyclo[2.1.1]hexan-4-yl)methoxy) quinazolin-4-yl)-3,8-diazabicyclo[3.2.1]octane-8-carboxylate

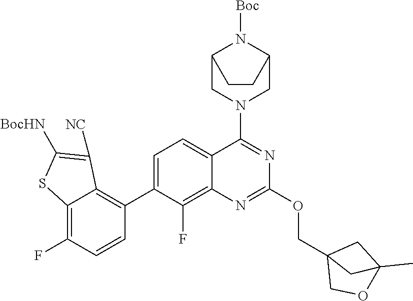 [SMILES: CC(C)(C)OC(=O)Nc1sc2c(F)ccc(-c3ccc4c(N5CC6CCC(C5)N6C(=O)OC(C)(C)C)nc(OCC56COC(C)(C5)C6)nc4c3F)c2c1C#N]

To a solution of (1-methyl-2-oxabicyclo[2.1.1]hexan-4-yl)methanol (19 mg, 0.15 mmol) in THE (2 mL) was added sodium hydride (6 mg, 0.15 mmol) at 0° C. The reaction mixture was stirred at 0° C. for 0.5 h. Then, tert-butyl 3-(7-(2-((tert-butoxy carbonyl)amino)-3-cyano-7-fluorobenzo[b]thiophen-4-yl)-2,8-difluoroquinazolin-4-yl)-3,8-diazabicyclo[3.2.1]octane-8-carboxylate (50 mg, 0.075 mmol) was added to the reaction mixture and the mixture was stirred at room temperature for 2 h. After completion, the reaction mixture was quenched with water (10 mL), extracted by EtOAc (30 mL×3) and washed with saturated NaCl aqueous solution. The organic layer was dried over anhydrous Na₂SO₄, filtered and the filtrate was concentrated to give the residue. The residue was purified by silica gel column chromatography and eluted with 0-3% MeOH in DCM to give the title product (45 mg, 0.058 mmol). MS (ESI, m/e) [M+1]+775.3.

### Step 5: 4-(4-(3,8-diazabicyclo[3.2.1]octan-3-yl)-8-fluoro-2-((1-methyl-2-oxabicyclo[2.1.1]hexan-4-yl)methoxy)quinazolin-7-yl)-2-amino-7-fluorobenzo[b] thiophene-3-carbonitrile

To a solution of tert-butyl 3-(7-(2-((tert-butoxycarbonyl)amino)-3-cyano-7-fluorobenzo[b]thiophen-4-yl)-8-fluoro-2-((1-methyl-2-oxabicyclo[2.1.1]hexan-4-yl)methoxy)quinazolin-4-yl)-3,8-diazabicyclo[3.2.1]octane-8-carboxylate (45 mg, 0.058 mmol) in DCM (2 mL) was added TFA (2 mL) at room temperature. The resulting mixture was stirred at room temperature for 3 h. After completion, the reaction mixture was concentrated to give the residue which was dissolved in 3 mL DCM and the pH was adjusted to about 10 using a solution of 7M NH₃ in MeOH. The residue was purified pre-HPLC to give the title product (12 mg, 0.021 mmol, Formate). ¹H NMR (500 MHz, CD₃OD) δ 8.53 (s, 0.5H), 7.81-7.80 (m, 1H), 7.26-7.23 (m, 2H), 7.04-7.00 (m, 1H), 4.73-4.48 (m, 4H), 3.92 (s, 2H), 3.80 (s, 2H), 3.72-3.63 (m, 2H), 2.11-1.91 (m, 4H), 1.81 (s, 2H), 1.64-1.62 (m, 2H), 1.43 (s, 3H). 8 MS (ESI, m/e) [M+1]+575.3.

### Example 16: 4-(4-(3-oxa-7,9-diazabicyclo[3.3.1]nonan-9-yl)-8-fluoro-2-((1-methyl-2-oxabicyclo[2.1.1]hexan-4-yl)methoxy)-6-(trifluoromethyl)quinazolin-7-yl)-2-amino-7-fluorobenzo[b]thiophene-3-carbonitrile

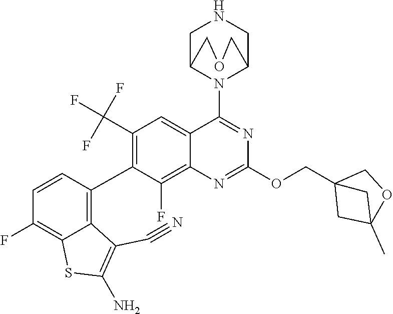 [SMILES: CC12CC(COc3nc(N4C5CNCC4COC5)c4cc(C(F)(F)F)c(-c5ccc(F)c6sc(N)c(C#N)c56)c(F)c4n3)(CO1)C2]

### Step 1: tert-butyl 9-(7-bromo-2-chloro-8-fluoro-6-(trifluoromethyl)quinazolin-4-yl)-3-oxa-7,9-diazabicyclo[3.3.1]nonane-7-carboxylate

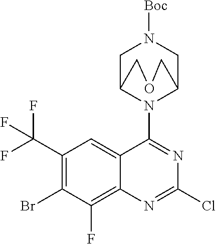 [SMILES: CC(C)(C)OC(=O)N1CC2COCC(C1)N2c1nc(Cl)nc2c(F)c(Br)c(C(F)(F)F)cc12]

To a mixture of 7-bromo-2,4-dichloro-8-fluoro-6-(trifluoromethyl)quinazoline (3.0 g, 8.2 mmol) and DIPEA (1.71 g, 13.14 mmol) in DCM (50 mL) was cooled to 0° C. Then, tert-butyl 3-oxa-7,9-diazabicyclo[3.3.1]nonane-7-carboxylate was added. The mixture was stirred for 2 h at 0° C., diluted with DCM (100 mL), washed with brine (20 mL), repeated operation three times and filtered. The filtrate was concentrated and purified by chromatography to give the title product (3.79 g, 83.1%). MS (ESI, m/e) [M+1]+556.4.

### Step 2: tert-butyl 9-(7-bromo-2,8-difluoro-6-(trifluoromethyl)quinazolin-4-yl)-3-oxa-7,9-diazabicyclo[3.3.1]nonane-7-carboxylate

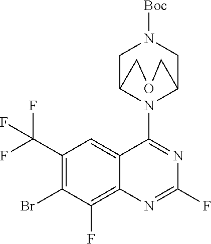 [SMILES: CC(C)(C)OC(=O)N1CC2COCC(C1)N2c1nc(F)nc2c(F)c(Br)c(C(F)(F)F)cc12]

To a solution of tert-butyl 9-(7-bromo-2-chloro-8-fluoro-6-(trifluoromethyl) quinazolin-4-yl)-3-oxa-7,9-diazabicyclo[3.3.1]nonane-7-carboxylate (3.6 g, 6.5 mmol) in DMSO (50 mL) was added KF (2.28 g, 39.1 mmol). The mixture was stirred at 90° C. for 2-3 h. The resulting mixture was cooled to rt, diluted with EtOAc (50 mL), washed with brine (20 mL) and the operation was repeated three times. The organic phase was filtered, and the filtrate was concentrated and purified by chromatography to give the title product (2.91 g. 83.1%). MS (ESI, m/e) [M+1]⁺ 539.7.

### Step 3: tert-butyl 9-(7-bromo-8-fluoro-2-((1-methyl-2-oxabicyclo[2.1.1]hexan-4-yl)methoxy)-6-(trifluoromethyl)quinazolin-4-yl)-3-oxa-7,9-diazabicyclo[3.3.1]nonane -7-carboxylate

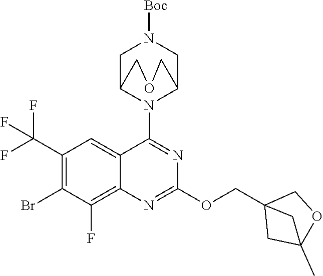 [SMILES: CC(C)(C)OC(=O)N1CC2COCC(C1)N2c1nc(OCC23COC(C)(C2)C3)nc2c(F)c(Br)c(C(F)(F)F)cc12]

A solution of (1-methyl-2-oxabicyclo[2.1.1]hexan-4-yl)methanol (95 mg, 0.74 mmol) in THE (30 mL) was cooled to 0° C. NaH (29 mg, 0.74 mmol) was added in one portion to the solution and the mixture was stirred at rt for 30 min. Then, tert-butyl 9-(7-bromo-2,8-difluoro-6-(trifluoromethyl)quinazolin-4-yl)-3-oxa-7,9-diazabicyclo[3.3.1]nonane-7-carboxylate (398 mg, 0.71 mmol) was added into the mixture. The resulting mixture was stirred at rt for 4-5 hours, then quenched with H₂O (20 mL), extracted with EtOAc (30 mL) for three times, washed with brine (20 mL) for three times, dried over anhydrous Na₂SO₄ and filtered. The filtrate was concentrated and purified by column chromatography to give the title product (327 mg, 71.08%). MS (EST, m/e) [M+H]⁺ 647.9.

### Step 4: tert-butyl 9-(7-(2-((tert-butoxycarbonyl)amino)-3-cyano-7-fluorobenzo [b]thiophen-4-yl)-8-fluoro-2-((1-methyl-2-oxabicyclo[2.1.1]hexan-4-yl)methoxy)-6-(trifluoromethyl)quinazolin-4-yl)-3-oxa-7,9-diazabicyclo[3.3.1]nonane-7-carboxylate

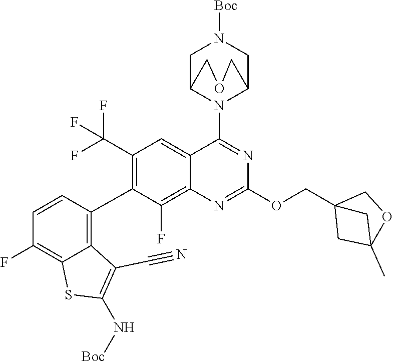 [SMILES: CC(C)(C)OC(=O)Nc1sc2c(F)ccc(-c3c(C(F)(F)F)cc4c(N5C6COCC5CN(C(=O)OC(C)(C)C)C6)nc(OCC56COC(C)(C5)C6)nc4c3F)c2c1C#N]

A mixture of tert-butyl 9-(7-bromo-8-fluoro-2-((1-methyl-2-oxabicyclo[2.1.1] hexan-4-yl)methoxy)-6-(trifluoromethyl)quinazolin-4-yl)-3-oxa-7,9-diazabicyclo[3.3.1]nonane-7-carboxylate (40 mg, 0.061 mmol), tert-butyl (3-cyano-7-fluoro-4-(4,4,5,5-tetramethyl-1,3,2-dioxaborolan-2-yl)benzo[b]thiophen-2-yl)carbamate (104 mg, 0.024 mmol), Cs₂CO₃ (60 mg, 0.18 mmol) and DPEphosPdCl₂ (10 mg, 0.012) in toluene (10 mL) was degassed with N₂ for 3 times and stirred at 100° C. for 16 hours. The resulting mixture was filtered, and the filtrate was concentrated. The residue was purified by column chromatography to give the title product (15 mg, 28.8%), MS (EST, m/e) [M+H]⁺ 859.2.

### Step 5: 4-(4-(3-oxa-7,9-diazabicyclo[3.3.1]nonan-9-yl)-8-fluoro-2-((1-methyl-2-oxabicyclo[2.1.1]hexan-4-yl)methoxy)-6-(trifluoromethyl)quinazolin-7-yl)-2-amino-7-fluorobenzo[b]thiophene-3-carbonitrile

To a solution of tert-butyl 9-(7-(2-((tert-butoxycarbonyl)amino)-3-cyano-7-fluorobenzo[b]thiophen-4-yl)-8-fluoro-2-((1-methyl-2-oxabicyclo[2.1.1]hexan-4-yl)methoxy)-6-(trifluoromethyl quinazolin-4-yl)-3-oxa-7,9-diazabicyclo[3.3.1] nonane-7-carboxylate (50 mg) in DCM (10 mL) was added TFA (2 mL). The mixture was stirred at room temperature for 2-3 hours. The resulting mixture was concentrated to dryness, then diluted with DCM/MeOH=10/1 (5 mL), added 5 drops of 7 M NH₃ in MeOH solution. Next, the new solution was concentrated to dryness and purified by column chromatography to give the title product (21 mg, 55.2%). ¹H NMR (500 MHz, CD₃OD) δ 7.96 (s, 1H), 7.22-7.20 (m, 1H), 7.03-6.99 (m, 1H), 4.74-4.72 (m, 2H), 4.46 (s, 2H), 4.31-4.10 (m, 4H), 3.79 (s, 2H), 3.53-3.37 (m, 4H), 1.81-1.80 (m, 2H), 1.69-1.53 (m, 2H), 1.42 (s, 3H). MS (ESI, m/e) [M+H]⁺ 659.8.

### Example 17: 4-(4-(3,8-diazabicyclo[3.2.1]octan-3-yl)-2-(3-(cyanomethyl)-3-methylazetidin-1-yl)-8-fluoro-6-trifluoromethyl)quinazolin-7-yl)-2-amino-7-fluoro benzo[b]thiophene-3-carbonitrile

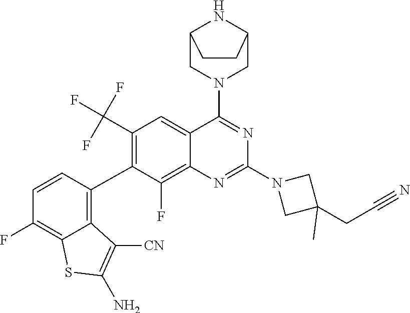 [SMILES: CC1(CC#N)CN(c2nc(N3CC4CCC(C3)N4)c3cc(C(F)(F)F)c(-c4ccc(F)c5sc(N)c(C#N)c45)c(F)c3n2)C1]

### Step 1: tert-butyl 3-(7-(2-((tert-butoxycarbonyl)amino)-7-fluorobenzo[b]thiophen-4-yl)-2,8-difluoro-6-(trifluoromethyl)quinazolin-4-yl)-3,8-diazabicyclo[3.2.1]octane-8-carboxylate

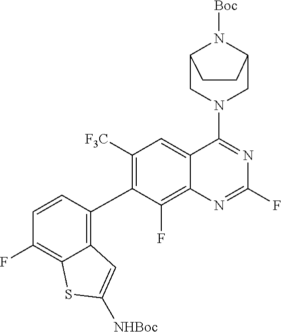 [SMILES: CC(C)(C)OC(=O)Nc1cc2c(-c3c(C(F)(F)F)cc4c(N5CC6CCC(C5)N6C(=O)OC(C)(C)C)nc(F)nc4c3F)ccc(F)c2s1]

Into a 1 L round-bottom flask, tert-butyl 3-(7-bromo-2,8-difluoro-6-(trifluoromethyl)quinazolin-4-yl)-3,8-diazabicyclo[3.2.1]octane-8-carboxylate (50 g, 0.097 mol), tert-butyl (4-(5,5-dimethyl-1,3,2-dioxaborinan-2-yl)-7-fluorobenzo[b]thiophen-2-yl)carbamate (47.2 g, 0.11 mol), Pd(dtbpf)Cl₂ (6.2 g, 0.096 mol), K₃PO₄ (36.5 g, 0.17 mol), dioxane (500 mL), H₂O (50 mL) were placed at room temperature. The resulting mixture was stirred for 2 h at 90° C. under N₂ atmosphere. The resulting mixture was extracted with EtOAc (2 L). The combined organic layers were dried over anhydrous Na₂SO₄. After filtration, the filtrate was concentrated under reduced pressure. The residue was purified by silica gel column chromatography, eluted with petroleum ether:EtOAc=100:0˜70:30 to give the title product (26 g, 38.2% yield). MS (ESI, m/e) [M+H]⁺=710.10.

### Step 2: tert-butyl 3-(7-(2-((tert-butoxycarbonyl)amino)-7-fluoro-3-iodobenzo[b]thiophen-4-yl)-2,8-difluoro-6-(trifluoromethyl)quinazolin-4-yl)-3,8-diazabicyclo[3.2.1]octane-8-carboxylate

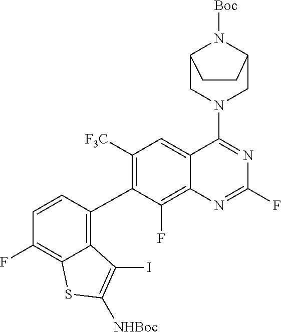 [SMILES: CC(C)(C)OC(=O)Nc1sc2c(F)ccc(-c3c(C(F)(F)F)cc4c(N5CC6CCC(C5)N6C(=O)OC(C)(C)C)nc(F)nc4c3F)c2c1I]

Into a 1 L round-bottom flask, tert-butyl 3-(7-(2-((tert-butoxycarbonyl)amino)-7-fluorobenzo[b]thiophen-4-yl)-2,8-difluoro-6-(trifluoromethyl)quinazolin-4-yl)-3,8-diazabicyclo[3.2.1]octane-8-carboxylate (39 g, 0.055 mol), CH₃CN (400 mL), NIS (16 g, 0.07 mol) were placed at room temperature. The resulting mixture was stirred for 2 h at room temperature. The resulting mixture was concentrated under vacuum. The residue was purified by silica gel column chromatography, eluted with petroleum ether:EtOAc=100: 0-70:30 to give the title product (20 g, 43.5% yield). MS (ESI, m/e) [M+H]⁺=836.00.

### Step 3: tert-butyl 3-(7-(2-amino-3-cyano-7-fluorobenzo[b]thiophen-4-yl)-2,8-difluoro-6-(trifluoromethyl)quinazolin-4-yl)-3,8-diazabicyclo[3.2.1]octane-8-carboxylate

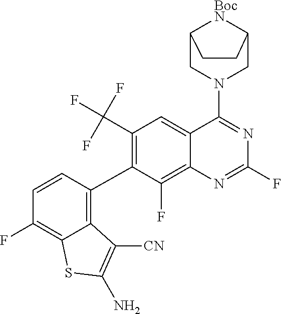 [SMILES: CC(C)(C)OC(=O)N1C2CCC1CN(c1nc(F)nc3c(F)c(-c4ccc(F)c5sc(N)c(C#N)c45)c(C(F)(F)F)cc13)C2]

To a 20-mL microwave tube was added tert-butyl 3-(7-(2-((tert-butoxy carbonyl)amino)-7-fluoro-3-iodobenzo[b]thiophen-4-yl)-2,8-difluoro-6-(trifluoro methyl)quinazolin-4-yl)-3,8-diazabicyclo[3.2.1]octane-8-carboxylate (100 mg, 0.12 mmol), Zn(CN)₂ (70 mg, 0.6 mmol), Pd(PPh₃)₄ (83 mg, 0.07 mmol) and DMF (10 mL) at room temperature under N₂ atmosphere. The resulting mixture was stirred for 10 min by microwave at 150° C. The resulting mixture was diluted with water (30 mL) and extracted by EtOAc (2×30 mL). The combined organic layers were washed with brine (3×30 mL) and dried over anhydrous Na₂SO₄. The organic layers were concentrated under reduced pressure. The residue was purified by TLC (Petroleum ether:EtOAc=1:1) to give the title product (25 mg, 32.9% yield). MS (ESI, m/e) [M+H]⁺=635.05.

### Step 4: tert-butyl 3-(7-(2-amino-3-cyano-7-fluorobenzo[b]thiophen-4-yl)-2-(3-(cyanomethyl)-3-methylazetidin-1-yl)-8-fluoro-6-(trifluoromethyl)quinazolin-4-yl)-3,8-diazabicyclo[3.2.1]octane-8-carboxylate

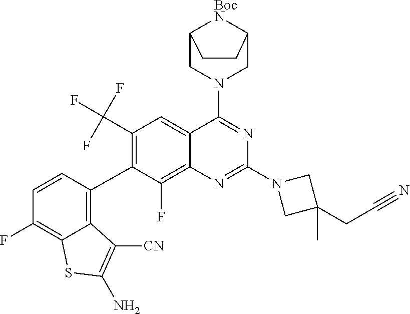 [SMILES: CC1(CC#N)CN(c2nc(N3CC4CCC(C3)N4C(=O)OC(C)(C)C)c3cc(C(F)(F)F)c(-c4ccc(F)c5sc(N)c(C#N)c45)c(F)c3n2)C1]

To a 10 mL vial was added tert-butyl 3-(7-(2-amino-3-cyano-7-fluorobenzo[b] thiophen-4-yl)-2,8-difluoro-6-(trifluoromethyl)quinazolin-4-yl)-3,8-diazabicyclo[3.2.1]octane-8-carboxylate (20 mg, 0.03 mmol), THE (1 mL), 2-(3-methylazetidin-3-yl)acetonitrile TFA salt (17 mg, 0.15 mmol) and DIEA (41 mg, 0.3 mmol) at rt. The mixture was stirred for 2 h at rt. The mixture was concentrated to give the title crude product (40 mg, crude). MS (ESI, m/e) [M+H]⁺ 725.15.

### Step 5: 4-(4-(3,8-diazabicyclo[3.2.1]octan-3-yl)-2-(3-(cyanomethyl)-3-methyl azetidin-1-yl)-8-fluoro-6-(trifluoromethyl)quinazolin-7-yl)-2-amino-7-fluorobenzo[b] thiophene-3-carbonitrile

To a 10 mL vial was added tert-butyl 3-(7-(2-amino-3-cyano-7-fluorobenzo[b] thiophen-4-yl)-2-(3-(cyanomethyl)-3-methylazetidin-1-yl)-8-fluoro-6-(trifluoro methyl)quinazolin-4-yl)-3,8-diazabicyclo[3.2.1]octane-8-carboxylate (20 mg, 0.03 mmol), DCM (1 mL) and TFA (0.2 mL) at rt. The mixture was stirred for 2 h at rt. The reaction mixture was concentrated under reduced pressure. The residue was purified by reverse phase flash with the following conditions (Column: XBridge Shield RP18 OBD Column, 30*150 mm, 5 μm; Mobile Phase A: Water (10 mmol/L NH₄HCO₃+0.1% NH₃—H₂O), Mobile Phase B: ACN; Flow rate: 60 mL/min; Gradient: 20% B to 50% B in 9 min, 50% B; Wavelength: 254 nm) to give the title product (5.1 mg, 29.6% yield, TFA salt). ¹H NMR (300 MHz, DMSO-d6) δ 9.14-9.13 (m, 1H), 8.94 (s, 1H), 8.06 (s, 2H), 7.90 (s, 1H), 7.22-7.08 (m, 2H), 4.50-4.09 (m, 6H), 3.99-3.89 (m, 4H), 2.96 (s, 2H), 1.94 (s, 4H), 1.41 (s, 3H). F NMR: 8-57.00, -74.23, -116.68, -124.64. MS (EST, m/e) [M+H]⁺ 625.10.

### Example 18: 4-(4 (3,8-diazabicyclo[3.2.1]octan-3-yl)-8-fluoro-2-((3-methyl oxetan-3-yl)methoxy)-6-(trifluoromethyl)quinazolin-7-yl)-2-amino-7-fluorobenzo [b]thiophene-3-carbonitrile

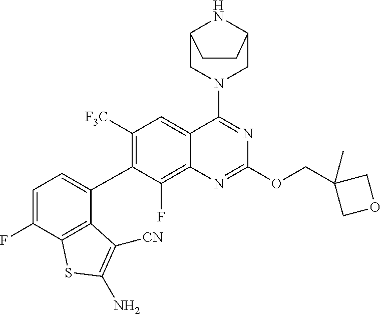 [SMILES: CC1(COc2nc(N3CC4CCC(C3)N4)c3cc(C(F)(F)F)c(-c4ccc(F)c5sc(N)c(C#N)c45)c(F)c3n2)COC1]

Example 18 was prepared by similar procedure as described in Example 3 by replacing (3-methoxytetrahydrofuran-3-yl)methanol with (3-methyloxetan-3-yl) methanol to give the title product (12.75 mg,). ¹H NMR (500 MHz, CD₃OD) δ 8.12 (s, 1H), 7.33-7.13 (m, 1H), 7.02-6.99 (m, 1H), 4.68-4.46 (m, 8H), 3.73-3.65 (m, 4H), 1.85 (s, 4H), 1.46 (s, 3H). MS (EST, m/e) [M+H]⁺ 617.5.

### Example 19: 4-(4-(3,8-diazabicyclo[3.2.1]octan-3-yl)-8-fluoro-2-((4-hydroxy tetrahydro-2H-pyran-4-yl)methoxy)-6-(trifluoromethyl)quinazolin-7-yl)-2-amino-7-fluorobenzo[b]thiophene-3-carbonitrile

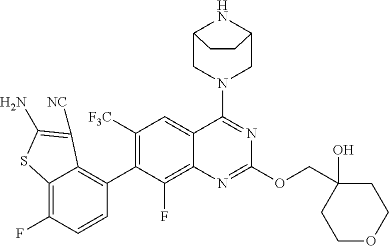 [SMILES: N#Cc1c(N)sc2c(F)ccc(-c3c(C(F)(F)F)cc4c(N5CC6CCC(C5)N6)nc(OCC5(O)CCOCC5)nc4c3F)c12]

Example 19 was prepared by similar procedure as described in Example 1 (step 6 and step 7) by replacing (1-(difluoromethyl)-2-oxabicyclo[2.1.1]hexan-4-yl) methanol with 4-(hydroxymethyl)tetrahydro-2H-pyran-4-ol to give the title product (6.4 mg). ¹H NMR (500 MHz, CD₃OD) δ 8.12 (s, 1H), 7.23-7.22 (m, 1H), 7.04-6.99 (m, 1H), 4.85-4.62 (m, 2H), 4.37-4.36 (m, 2H), 4.24-4.22 (m, 2H), 3.93-3.75 (m, 6H), 2.18-2.13 (m, 4H), 1.87-1.63 (m, 4H). MS (ESI, m/e) [M+H]⁺ 647.5.

### Example 20: methyl 3-(((7-(2-amino-3-cyano-7-fluorobenzo[b]thiophen-4-yl)-4-(3,8-diazabicyclo[3.2.1]octan-3-yl)-8-fluoro-6-(trifluoromethyl)quinazolin-2-yl)oxy) methyl)-3-methoxyazetidine-1-carboxylate

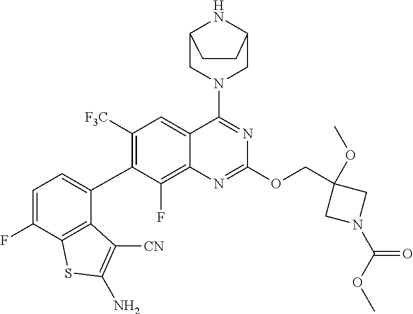 [SMILES: COC(=O)N1CC(COc2nc(N3CC4CCC(C3)N4)c3cc(C(F)(F)F)c(-c4ccc(F)c5sc(N)c(C#N)c45)c(F)c3n2)(OC)C1]

Example 20 was prepared by similar procedure as described in Example 8 by replacing (3-methylazetidin-3-yl)methanol hydrochloride with (3-methoxyazetidin-3-yl)methanol hydrochloride to give the title product (1 mg. 9% yield, Formate). ¹H NMR (500 MHz, CD₃OD) δ 8.51 (s, 1H), 8.17 (s, 1H). 7.29-7.21 (m, 1H), 7.03-7.00 (m, 1H), 4.81-4.76 (m, 2H), 4.72-4.61 (m, 2H), 4.12-4.00 (m, 5H), 3.87-3.80 (m, 2H), 3.75-3.68 (m, 3H), 3.51-3.41 (m, 4H), 2.05 (s, 4H). MS (ESI, m/e) [M+1]+690.5.

### Example 21: 4-(4-(3,8-diazabicyclo[3.2.1]octan-3-yl)-8-fluoro-2-((1-(hydroxy methyl)-2-oxabicyclo[2.1.1]hexan-4-yl)methoxy)-6-(trifluoromethyl)quinazolin-7-yl)-2-amino-7-fluorobenzo[b]thiophene-3-carbonitrile

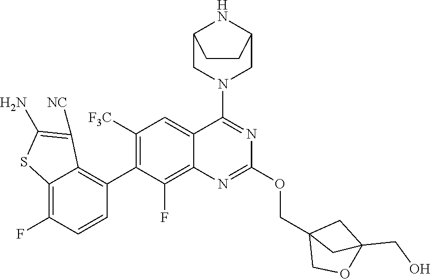 [SMILES: N#Cc1c(N)sc2c(F)ccc(-c3c(C(F)(F)F)cc4c(N5CC6CCC(C5)N6)nc(OCC56COC(CO)(C5)C6)nc4c3F)c12]

### Step 1: (4-(hydroxymethyl)-2-oxabicyclo[2.1.1]hexan-1-yl)methyl acetate

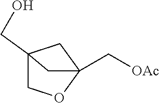 [SMILES: CC(=O)OCC12CC(CO)(CO1)C2]

To a solution of (1-(iodomethyl)-2-oxabicyclo[2.1.1]hexan-4-yl)methanol (254 mg, 1.0 mmol) in DMSO (20 mL) was added potassium acetate (980.0 mg, 10.0 mmol) at room temperature. The resulting mixture was stirred at 100° C. for 16 h. After completion, the reaction mixture was diluted with water (30 mL), extracted with EtOAc (100 mL) and washed with saturated NaCl (50 mL×3) aqueous solution. The organic layer was dried over anhydrous Na₂SO₄, filtered and the filtrate was concentrated to give the crude product (50 mg).

Example 21 was prepared by similar procedure as described in Example 1 (step 6 and step 7) by replacing (1-(difluoromethyl)-2-oxabicyclo[2.1.1]hexan-4-yl) methanol with ((4-(hydroxymethyl)-2-oxabicyclo[2.1.1]hexan-1-yl)methyl acetate to give the title product (0.8 mg, Formate). ¹H NMR (500 MHz, CD₃OD) δ 8.50 (s, 1H), 8.11 (s, 1H), 7.24-7.16 (m, 1H), 7.03-7.00 (m, 1H), 4.80-4.46 (m, 4H), 3.91-3.65 (m, 8H), 2.16-1.78 (m, 8H). MS (ESI, m/e) [M+H]⁺ 659.4

### Example 22: 4-(((7-(2-amino-3-cyano-7-fluorobenzo[b]thiophen-4-yl)-4-(3,8-diazabicyclo[3.2.1]octan-3-yl)-8-fluoro-6-(trifluoromethyl)quinazolin-2-yl)oxy) methyl)tetrahydro-2H-pyran-4-carbonitrile

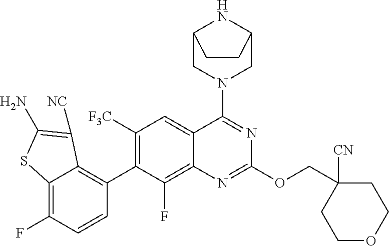 [SMILES: N#Cc1c(N)sc2c(F)ccc(-c3c(C(F)(F)F)cc4c(N5CC6CCC(C5)N6)nc(OCC5(C#N)CCOCC5)nc4c3F)c12]

Example 22 was prepared by similar procedure as described in Example 1 (step 6 and step 7) by replacing (1-(difluoromethyl)-2-oxabicyclo[2.1.1]hexan-4-yl) methanol with 4-(hydroxymethyl)tetrahydro-2H-pyran-4-carbonitrile to give the title product. It was separated by Chiral-HPLC (Prep-HPLC Equipment: Prep-HPLC-Gilson; Column: IE, 20 mm×250 mm, 5 μm; Mobile Phase A: n-Hexane, Mobile Phase B: EtOH (0.2% 2M NH₃ in MeOH); Flow Rate: 18 mL/min; Temperature: 25° C.; Gradient: 50% B in 14 min; Retention time—isomer 1: 7 min; Retention time -isomer 2: 11.5 min) to give isomer 1 (21.2 mg) and isomer 2 (20.2 mg). Isomer 1: ¹H NMR (500 MHz, CD₃OD) δ 8.13 (s, 1H) 7.22-7.20 (m, 1H), 7.02-6.99 (m, 1H), 4.57-4.45 (m, 4H), 4.01-3.99 (m, 2H), 3.74-3.64 (m, 6H), 2.06-2.03 (m, 2H), 1.87-1.82 (m, 6H); MS (ESI, m/e) [M+H]⁺ 656.5. Isomer 2: ¹H NMR (500 MHz, CD₃OD) δ 8.13 (s, 1H), 7.22-7.20 (m, 1H), 7.02-6.99 (m, 1H), 4.57-4.45 (m, 4H), 4.01-3.99 (m, 2H), 3.74-3.62 (m, 6H), 2.06-2.03 (m, 2H), 1.87-1.82 (m, 6H); MS (EST, m/e) [M+H]⁺ 656.5.

### Example 23: 4-(4-(3,8-diazabicyclo[3.2.1]octan-3-yl)-8-fluoro-2-((tetrahydro-2H-pyran-4-yl)methoxy)-6-(trifluoromethyl)quinazolin-7-yl)-2-amino-7-fluorobenzo[b]thiophene-3-carbonitrile

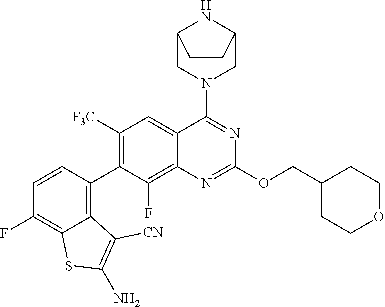 [SMILES: N#Cc1c(N)sc2c(F)ccc(-c3c(C(F)(F)F)cc4c(N5CC6CCC(C5)N6)nc(OCC5CCOCC5)nc4c3F)c12]

### Step 1: tert-butyl 3-(7-bromo-8-fluoro-2-(methylthio)-6-(trifluoromethyl) quinazolin-4-yl)-3,8-diazabicyclo[3.2.1]octane-8-carboxylate

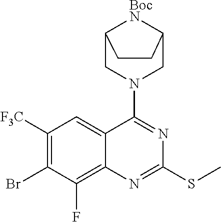 [SMILES: CSc1nc(N2CC3CCC(C2)N3C(=O)OC(C)(C)C)c2cc(C(F)(F)F)c(Br)c(F)c2n1]

The mixture of tert-butyl 3-(7-bromo-2,8-difluoro-6-(trifluoromethyl)quinazolin-4-yl)-3,8-diazabicyclo[3.2.1]octane-8-carboxylate (14.8 g, 28.3 mmol) and sodium methanethiolate (1.96 g. 28.4 mmol) in THE (500 mL) was stirred at room temperature for 15 h. The reaction mixture was diluted with EtOAc (50 mL) and washed with saturated NaCl (15 mL×3) aqueous solution. The organic layer was dried over anhydrous Na₂SO₄, filtered and the filtrate was concentrated to give the residue. The residue was purified by combi-flash (Petroleum ether:EtOAc=10:1) to give the title product (10.0 g, 64.1%). MS (EST, m/e) [M+1]+551.4.

### Step 2: tert-butyl 3-(7-(2-((tert-butoxycarbonyl)amino)-3-cyano-7-fluorobenzo[h] thiophen-4-yl)-8-fluoro-2-(methylthio)-6-(trifluoromethyl)quinazolin-4-yl)-3,8-diaza bicyclo[3.2.1]octane-8-carboxylate

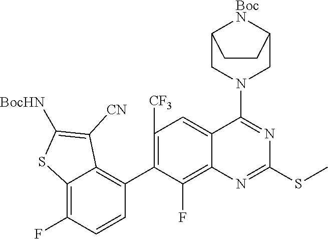 [SMILES: CSc1nc(N2CC3CCC(C2)N3C(=O)OC(C)(C)C)c2cc(C(F)(F)F)c(-c3ccc(F)c4sc(NC(=O)OC(C)(C)C)c(C#N)c34)c(F)c2n1]

The mixture of tert-butyl 3-(7-bromo-8-fluoro-2-(methylthio)-6-(trifluoromethyl) quinazolin-4-yl)-3,8-diazabicyclo[3.2.1]octane-8-carboxylate (5.5 g mg, 10 mmol), tert-butyl (3-cyano-7-fluoro-4-(4,4,5,5-tetramethyl-1,3,2-dioxaborolan-2-yl)benzo[b] thiophen-2-yl)carbamate (8.36 g, 20 mmol). Pd(dtbpf)Cl₂ (3.26 g, 5 mmol) and K₃PO₄ (6.36 g, 30 mmol) in dioxane/water (550 mL/55 mL) was stirred at 80° C. for 0.5 h under N₂ atmosphere. After completion, the reaction mixture was diluted with EtOAc (50 mL) and washed with saturated NaCl (15 mL×3) aqueous solution. The organic layer was dried over anhydrous Na₂SO₄, filtered and the filtrate was concentrated to give the residue. The residue was purified by combi-flash (Petroleum ether:EtOAc=3:1) to give the title product (2.9 g, 38.1%). MS (EST, m/e) [M+1]⁺ 763.5.

### Step 3: tert-butyl 3-(7-(2-((tert-butoxycarbonyl)amino)-3-cyano-7-fluorobenzo[b] thiophen-4-yl)-8-fluoro-2-(methylsulfonyl)-6-(trifluoromethyl)quinazolin-4-yl)-3,8-diazabicyclo[3.2.1]octane-8-carboxylate

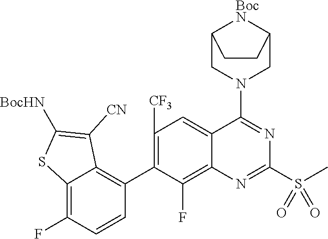 [SMILES: CC(C)(C)OC(=O)Nc1sc2c(F)ccc(-c3c(C(F)(F)F)cc4c(N5CC6CCC(C5)N6C(=O)OC(C)(C)C)nc(S(C)(=O)=O)nc4c3F)c2c1C#N]

The mixture of tert-butyl 3-(7-(2-((tert-butoxycarbonyl)amino)-3-cyano-7-fluoro benzo[b]thiophen-4-yl)-8-fluoro-2-(methylthio)-6-(trifluoromethyl)quinazolin-4-yl)-3,8-diazabicyclo[3.2.1]octane-8-carboxylate (1.24 g, 1.63 mmol) and m-CPBA (561 mg, 3.26 mmol) in DCM (10 mL) was stirred at room temperature for 3 h. The reaction mixture was diluted with EtOAc (50 mL) and washed with saturated NaHCO₃ aqueous solution (15 mL×3). The organic layer was dried over anhydrous Na₂SO₄, filtered and the filtrate was concentrated to give the residue. The residue was directly used in the next step without further purification. MS (EST, m/e) [M+1]⁺ 795.6.

### Step 4: tert-butyl 3-(7-(2-((tert-butoxycarbonyl)amino)-3-cyano-7-fluorobenzo[h] thiophen-4-yl)-8-fluoro-2-((tetrahydro-2H-pyran-4-yl)methoxy)-6-(trifluoromethyl) quinazolin-4-yl)-3,8-diazabicyclo[3.2.1]octane-8-carboxylate

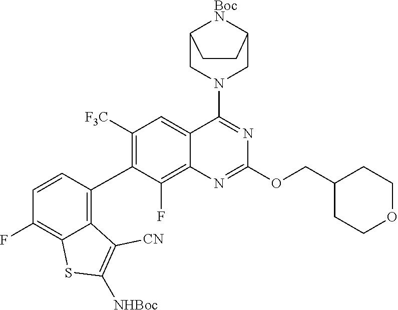 [SMILES: CC(C)(C)OC(=O)Nc1sc2c(F)ccc(-c3c(C(F)(F)F)cc4c(N5CC6CCC(C5)N6C(=O)OC(C)(C)C)nc(OCC5CCOCC5)nc4c3F)c2c1C#N]

To a solution of (tetrahydro-2H-pyran-4-yl)methanol (12 mg, 0.1 mmol) in THE (2 mL) was added LiHMDS (0.1 mL, 0.1 mmol) at 0° C. The reaction mixture was stirred at room temperature for 0.5 h. Then, tert-butyl 3-(7-(2-((tert-butoxycarbonyl) amino)-3-cyano-7-fluorobenzo[b]thiophen-4-yl)-8-fluoro-2-(methylsulfonyl)-6-(trifluoromethyl)quinazolin-4-yl)-3,8-diazabicyclo[3.2.1]octane-8-carboxylate (40 mg, 0.05 mmol) was added to the reaction mixture and the mixture was stirred at room temperature for 1 h. After completion, the reaction mixture was diluted with EtOAc (40 mL) and washed with saturated NaCl (15 mL×3) aqueous solution. The organic layer was dried over anhydrous Na₂SO₄, filtered and the filtrate was concentrated to give the residue. The residue was purified by Prep-TLC (Petroleum ether:EtOAc=2:1) to give the title product (35 mg, 85.4%). MS (ESI, m/e) [M+1]⁺ 831.6.

### Step 5: 4-(4-(3,8-diazabicyclo[3.2.1]octan-3-yl)-8-fluoro-2-(tetrahydro-2H-pyran-4-yl)methoxy)-6-(trifluoromethyl)quinazolin-7-yl)-2-amino-7-fluorobenzo [b]thiophene-3-carbonitrile

To a solution of tert-butyl 3-(7-(2-((tert-butoxycarbonyl)amino)-3-cyano-7-fluorobenzo[b]thiophen-4-yl)-8-fluoro-2-((tetrahydro-2H-pyran-4-yl)methoxy)-6-(trifluoromethyl)quinazolin-4-yl)-3,8-diazabicyclo[3.2.1]octane-8-carboxylate (35 mg, 0.042 mmol) in DCM (2 mL) was added TFA (2 mL) at room temperature. The resulting mixture was stirred at room temperature for 3 h. After completion, the reaction mixture was concentrated to give the residue. The residue was purified by prep-HPLC to give the title product (9.79 mg, 36.9%). ¹H NMR (500 MHz, CD₃OD) δ 8.10 (s. 1H), 7.28-7.15 (m, 1H), 7.03-6.99 (m, 1H), 4.66-4.52 (m, 2H), 4.34-4.32 (m, 2H), 4.00-3.91 (m, 4H). 3.82-3.72 (m, 2H), 3.53-3.44 (m, 2H), 2.14-2.11 (m, 1H), 2.04-1.94 (m, 4H). 1.79-1.77 (m, 2H), 1.56-1.37 (m, 2H). MS (EST, m/e) [M+1]⁺ 631.5.

### Example 24: 4-(4-(3,8-diazabicyclo[3.2.1]octan-3-yl)-8-fluoro-2-((1-(1-methyl-1H-imidazol-2-yl)azetidin-3-yl)methoxy)-6-(trifluoromethyl)quinazolin-7-yl)-2-amino-7-fluorobenzo[b]thiophene-3-carbonitrile

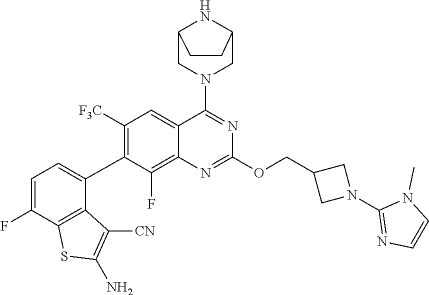 [SMILES: Cn1ccnc1N1CC(COc2nc(N3CC4CCC(C3)N4)c3cc(C(F)(F)F)c(-c4ccc(F)c5sc(N)c(C#N)c45)c(F)c3n2)C1]

### Step 1: (1-(1-methyl-1H-imidazol-2-yl)azetidin-3-yl)methanol

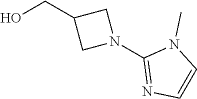

A solution of azetidin-3-ylmethanol hydrochloride (500 mg, 4.06 mmol), 2-iodo-1-methyl-1H-imidazole (527 mg, 4.87 mmol), CuI (78 mg, 0.41 mmol), (R)-proline (94 mg, 10.82 mmol) and Cs₂CO₃ (2.65 g, 8.12 mmol) in DMF (30 mL) was stirred at 100° C. for 3 h. The solvent was concentrated, and the residue was purified by column chromatography on silica gel (DCM:MeOH=15:1) to give the title product (220 mg, 32%). MS (EST, m/e) [M+1]⁺ 168.4.

Example 24 was prepared by similar procedure as described in Example 8 by replacing methyl 3-(hydroxymethyl)-3-methylazetidine-1-carboxylate with (1-(1-methyl-1H-imidazol-2-yl)azetidin-3-yl)methanol to give the title product (5 mg, 14% yield, Formate). ¹H NMR (500 MHz, CD₃OD) δ 8.49 (s, 1H), 8.12 (s, 1H), 7.20-7.22 (m, 1H), 7.06-7.00 (m, 1H), 6.74 (s, 1H), 6.69 (s, 1H). 4.52-4.73 (m, 4H), 4.32-4.26 (m, 2H), 4.12-4.05 (m, 2H), 3.94-3.89 (m, 2H), 3.81-3.72 (m, 2H), 3.44 (s, 3H), 3.30-3.15 (m, 1H), 1.95 (s, 4H). MS (EST, m/e) [M+1]⁺ 682.5.

### Example 25: 4-(4-(3,8-diazabicyclo[3.2.1]octan-3-yl)-8-fluoro-2-((3-fluorotetra hydrofuran-3-yl)methoxy)-6-(trifluoromethyl)quinazolin-7-yl)-2-amino-7-fluoro benzo[b]thiophene-3-carbonitrile

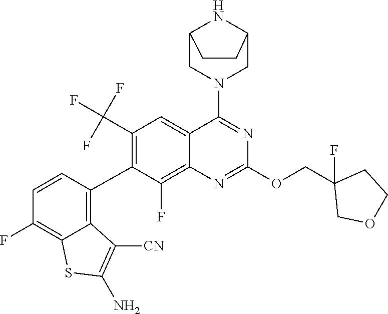 [SMILES: N#Cc1c(N)sc2c(F)ccc(-c3c(C(F)(F)F)cc4c(N5CC6CCC(C5)N6)nc(OCC5(F)CCOC5)nc4c3F)c12]

### Step 1: tert-butyl 3-(7-(2-((tert-butoxycarbonyl)amino)-3-cyano-7-fluorobenzo [b]thiophen-4-yl)-8-fluoro-2-((3-fluorotetrahydrofuran-3-yl)methoxy)-6-(trifluoro methyl)quinazolin-4-yl)-3,8-diazabicyclo[3.2.1]octane-8-carboxylate

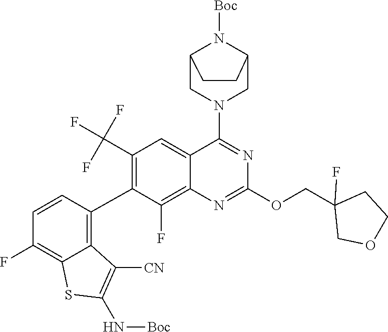 [SMILES: CC(C)(C)OC(=O)Nc1sc2c(F)ccc(-c3c(C(F)(F)F)cc4c(N5CC6CCC(C5)N6C(=O)OC(C)(C)C)nc(OCC5(F)CCOC5)nc4c3F)c2c1C#N]

To a stirred mixture of (3-fluorotetrahydrofuran-3-yl)methanol (8.2 mg, 0.07 mmol) and NaH (3.3 mg, 0.14 mmol) in THE (1 ml) was added tert-butyl 3-(7-(2-((tert-butoxycarbonyl)amino)-3-cyano-7-fluorobenzo[b]thiophen-4-yl)-2,8-difluoro-6-(trifluoromethyl)quinazolin-4-yl)-3,8-diazabicyclo[3.2.1]octane-8-carboxylate (50 mg, 0.07 mmol) in portions at 0′C. The resulting mixture was stirred for 1h at room temperature. The reaction was quenched with saturated NH₄Cl aqueous solution. The resulting mixture was extracted with EtOAc (3×30 mL). The combined organic layers were washed with brine (50 mL) and dried over anhydrous Na₂SO₄. After filtration, the filtrate was concentrated under reduced pressure. The residue was purified by Prep-TLC (Petroleum ether/EtOAc 1:1) to give the title product (30 mg, 69.47% yield). MS (ESI, m/e) [M+H]⁺ 835.35.

Step 2: 4-(4-(3,8-diazabicyclo[3.2.1]octan-3-yl)-8-fluoro-2-((3-fluorotetrahydro furan-3-yl)methoxy)-6-(trifluoromethyl)quinazolin-7-yl)-2-amino-7-fluorobenzo[b] thiophene-3-carbonitrile

A solution of tert-butyl 3-(7-(2-((tert-butoxycarbonyl)amino)-3-cyano-7-fluoro benzo[b]thiophen-4-yl)-8-fluoro-2-((3-fluorotetrahydrofuran-3-yl)methoxy)-6-(tri fluoromethyl)quinazolin-4-yl)-3,8-diazabicyclo[3.2.1]octane-8-carboxylate (30 mg, 0.04 mmol) and TFA (0.5 mL) in DCM (1.5 ml) was stinted for 2 h at room temperature. The resulting mixture was concentrated under reduced pressure. The residue was purified by Prep-HPLC (Column: Xselect CSH C18 OBD Column 30*150 mm 5 μm; Mobile Phase A: Water (0.1% TFA), Mobile Phase B: ACN; Flow rate: 60 mL/min; Gradient: 15% B to 45% B in 8 min, 45% B; Wavelength: 254 nm; RT1(min): 7) to give the title product (8.1 mg. 35.52% yield, TFA salt/Formate). ¹H NMR (300 MHz, CD₃OD) δ 8.47 (s, 0.5H), 8.13 (s, 1H), 7.21 (s, 1H), 7.02 (s, 1H), 4.76-4.57 (m, 4H), 4.26-3.68 (m, 8H), 2.45-2.16 (m, 2H), 2.05 (s, 4H). MS (ESI, m/e) [M+H]⁺ 635.40.

### Example 26: 4-(4-(3,8-diazabicyclo[3.2.1]octan-3-yl)-8-fluoro-6-(trifluoro methyl)-2-((3-(trifluoromethyl)tetrahydrofuran-3-yl)methoxy)quinazolin-7-yl)-2-amino-7-fluorobenzo[b]thiophene-3-carbonitrile

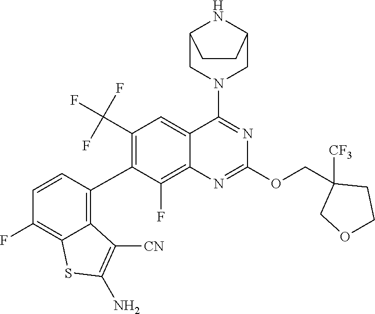 [SMILES: N#Cc1c(N)sc2c(F)ccc(-c3c(C(F)(F)F)cc4c(N5CC6CCC(C5)N6)nc(OCC5(C(F)(F)F)CCOC5)nc4c3F)c12]

### Step 1: diethyl 2-(2-(benzyloxy)ethyl)malonate

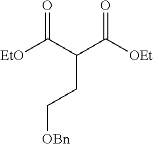 [SMILES: CCOC(=O)C(CCOCc1ccccc1)C(=O)OCC]

To a solution of ((2-bromoethoxy)methyl)benzene (40 g, 185.97 mmol) in THF (500 mL) was added NaH (8.93 g, 223.16 mmol, 60%) in portions at 0° C. After being stirred for 5 mins, diethyl malonate (60 g, 371.94 mmol) was added. The resulting mixture was stirred for 12 h at 0° C. The resulting mixture quenched with saturated NH₄Cl aqueous solution at 0° C. and extracted with EtOAc (3×200 mL). The combined organic layers were washed with brine (2×200 mL), dried over anhydrous Na₂SO₄. After filtration, the filtrate was concentrated under reduced pressure. The residue was purified by C18 column chromatography and eluted with H₂O: CH₃CN (100:0-20:80) to give the title product (44 g, 80.38% yield). MS (EST, m/e) [M+H]⁺ 295.10.

### Step 2: diethyl 2-(2-(benzyloxy)ethyl)-2-(trifluoromethyl)malonate

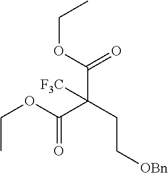 [SMILES: CCOC(=O)C(CCOCc1ccccc1)(C(=O)OCC)C(F)(F)F]

To a solution of diethyl 2-(2-(benzyloxy)ethyl)malonate (40 g, 135.89 mmol) in DMF (500 mL) was added NaH (6.52 g, 163.07 mmol, 60%) in portions at 0° C. After being stirred for 1 h at room temperature, 2,8-difluoro-5-(trifluoromethyl)-5H-dibenzo[b,d] thiophen-5-ium trifluoromethanesulfonate (87.24 g, 203.84 mmol) was added at −60° C. The resulting mixture was stirred for 12 h at room temperature. The resulting mixture was quenched with saturated NH₄Cl aqueous solution at 0° C. and extracted by EtOAc (3×200 mL). The combined organic layers were washed with brine (2×200 mL) and dried over anhydrous Na₂SO₄. After filtration, the filtrate was concentrated under reduced pressure. The residue was purified by silica gel column chromatography and eluted with petroleum ether:EtOAc (100: 0-80:20) to give the title product (10 g, 20.31% yield). MS (EST, m/e) [M+H]⁺ 363.10.

### Step 3: 2-(2-(benzyloxy)ethyl)-2-(trifluoromethyl)propane-1,3-diol

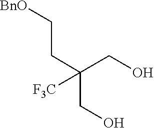 [SMILES: OCC(CO)(CCOCc1ccccc1)C(F)(F)F]

To a solution of diethyl 2-(2-(benzyloxy)ethyl)-2-(trifluoromethyl)malonate (9.5 g, 26.22 mmol) in THF (100 mL) was added LiAlH₄ (2.99 g, 78.65 mmol) in portions at 0° C. After being stirred for 1 h at room temperature, the resulting mixture was quenched with Na₂SO₄·10H₂O at 0° C. After filtration, the filtrate was concentrated under reduced pressure. The residue was purified by silica gel column chromatography and eluted with petroleum ether:EtOAc (100:0-60:40) to give the title product (2.1 g, 28.78% yield). MS (ESI, m/e) [M+H]⁺ 279.00.

### Step 4: 4-(benzyloxy)-2-(((tert-butyldiphenylsilyl)oxy)methyl)-2-(trifluoro methyl)butan-1-ol

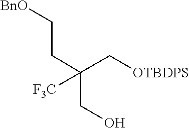

To a solution of 2-(2-(benzyloxy)ethyl)-2-(trifluoromethyl)propane-1,3-diol (2.1 g, 7.55 mmol) and 1H-imidazole (1.54 g, 22.64 mmol) in DCM (50 mL) was added TBDPSCl (2.28 g, 8.30 mmol) in portions at 0° C. After being stirred for 3 h at room temperature, the mixture was concentrated under reduced pressure. The residue was purified by silica gel column chromatography and eluted with petroleum ether:EtOAc (100:0-70:30) to give the title product (2.5 g, 64.12% yield). MS (ESI, m/e) [M+Na]⁺ 539.25.

### Step 5: 2-(((tert-butyldiphenylsilyl)oxy)methyl)-2-(trifluoromethyl)butane-1,4-diol

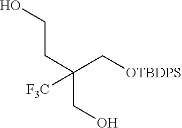

To a solution of 4-(benzyloxy)-2-(((tert-butyldiphenylsilyl)oxy)methyl)-2-(tri fluoromethyl)butan-1-ol (1.2 g, 2.323 mmol) in EtOH (50 mL) was Pd/C (1.2 g, 10%) at room temperature. After being stirred for 12 h at 80° C. under H₂ (g) atmosphere, the mixture was filtered, the filtrate was concentrated under reduced pressure. The residue was purified by silica gel column chromatography and eluted with petroleum ether:EtOAc (100:0-60:40) to give the title product (500 mg, 40.38% yield). MS (ESI, m/e) [M+H]⁺ 427.15.

### Step 6: tert-butyldiphenyl((3-(trifluoromethyl)tetrahydrofuran-3-yl)methoxy)silane

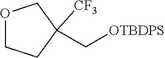

To a solution of 2-(((tert-butyldiphenylsilyl)oxy)methyl)-2-(trifluoromethyl) butane-1,4-diol (480 mg, 1.13 mmol) and TsCl (300.3 mg, 1.58 mmol) in THF (50 mL) was added n-BuLi (0.54 mL, 1.35 mmol, 2.5 M in hexane) at 0° C. After being stirred for 1.5 h at room temperature, n-BuLi (0.54 mL, 1.35 mmol, 2.5 M in hexane) was added at 0° C. The resulting mixture was stirred for 12 h at room temperature. The resulting mixture was quenched with saturated NH₄Cl aqueous solution at 0° C. and extracted by EtOAc (3×50 mL). The combined organic layers were washed with brine (2×20 mL) and dried over anhydrous Na₂SO₄. After filtration, the filtrate was concentrated under reduced pressure. The residue was purified by silica gel column chromatography and eluted with petroleum ether:EtOAc (100:0-90:10) to give the title product (190 mg, 41.33% yield).

### Step 7: (3-(trifluoromethyl)tetrahydrofuran-3-yl)methanol

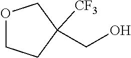 [SMILES: OCC1(C(F)(F)F)CCOC1]

To a solution of tert-butyldiphenyl((3-(trifluoromethyl)tetrahydrofuran-3-yl)methoxy)silane (90 mg, 0.22 mmol) in THE (50 mL) was added TBAF (0.55 mL, 0.55 mmol, 1 M in THF) at 0° C. After being stirred for 12 h at room temperature, the mixture was concentrated under reduced pressure. The residue was purified by silica gel column chromatography and eluted with petroleum ether:EtOAc (100:0-60:40) to give the title product (15 mg, 40.02%).

### Step 8: tert-butyl 3-(7-(2-((tert-butoxycarbonyl)amino)-3-cyano-7-fluorobenzo[b]thiophen-4-yl)-8-fluoro-6-(trifluoromethyl)-2-((3-(trifluoromethyl)tetrahydrofuran-3-yl)methoxy)quinazolin-4-yl)-3,8-diazabicyclo[3.2.1]octane-8-ca

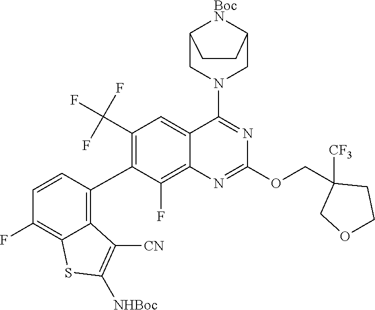 [SMILES: CC(C)(C)OC(=O)Nc1sc2c(F)ccc(-c3c(C(F)(F)F)cc4c(N5CC6CCC(C5)N6C(=O)OC(C)(C)C)nc(OCC5(C(F)(F)F)CCOC5)nc4c3F)c2c1C#N]

To a solution of tert-butyl 3-(7-(2-((tert-butoxycarbonyl)amino)-3-cyano-7-fluorobenzo[b] thiophen-4-yl)-2,8-difluoro-6-(trifluoromethyl)quinazolin-4-yl)-3,8-diazabicyclo[3.2.1]octane-8-carboxylate (20 mg, 0.03 mmol) and (3-(trifluoromethyl) tetrahydrofuran-3-yl)methanol (9.26 mg, 0.06 mmol) in THF (2 mL) was added NaH (2.18 mg, 0.06 mmol, 60%) at 0° C. After being stirred for 2 h at room temperature, the mixture was quenched with saturated NH₄Cl aqueous solution at 0° C. and extracted by EtOAc (3×20 mL). The combined organic layers were washed with brine (2×20 mL) and dried over anhydrous Na₂SO₄. After filtration, the filtrate was concentrated under reduced pressure. The residue was purified by Prep-TLC with petroleum ether:EtOAc (4:1) to give the title product (13 mg, 53.97% yield). MS (EST, m/e) [M+H]⁺ 885.25.

### Step 9: 4-(4-(3,8-diazabicyclo[3.2.1]octan-3-yl)-8-fluoro-6-(trifluoromethyl)-2-((3-(trifluoromethyl)tetrahydrofuran-3-yl)methoxy)quinazolin-7-yl)-2-amino-7-fluoro benzo[b] thiophene-3-carbonitrile

To a stirred solution of tert-butyl 3-(7-(2-((tert-butoxycarbonyl)amino)-3-cyano-7-fluorobenzo[b]thiophen-4-yl)-8-fluoro-6-(trifluoromethyl)-2-((3-(trifluoromethyl)tetrahydrofuran-3-yl)methoxy)quinazolin-4-yl)-3,8-diazabicyclo[3.2.1]octane-8-carboxylate (13 mg, 0.015 mmol) in DCM (0.6 ml) was added TFA (0.2 mL). The resulting mixture was stirred 1 h at room temperature and concentrated under reduced pressure. The crude product was purified by Prep-HPLC (Column: XBridge Shield RP18 OBD Column, 30*150 mm, 5 μm; Mobile Phase A: Water (0.05% TFA), Mobile Phase B: ACN; Flow rate: 60 mL/min; Gradient: 32% B to 62% B in 9 min, 62% B; Wavelength: 220 nm; RT1(min): 7) to give the title product (0.8 mg, 6.82% yield, TFA salt). ¹H NMR (300 MHz, CD₃OD) δ 8.13 (s, 1H), 7.24-7.20 (m, 1H), 7.09-6.98 (m, 1H), 4.81-4.54 (m, 4H), 4.23 (s, 2H), 4.04-3.83 (m, 6H), 2.39-2.05 (m, 6H). MS (EST, m/e) [M+H]⁺ 685.20.

### Example 27: 4-(4-(3,8-diazabicyclo[3.2.1]octan-3-yl)-8-fluoro-2-((4-hydroxy-4-methyltetrahydrofuran-3-yl)methoxy)-6-(trifluoromethyl)quinazolin-7-yl)-2-amino-7-fluorobenzo[b]thiophene-3-carbonitrile

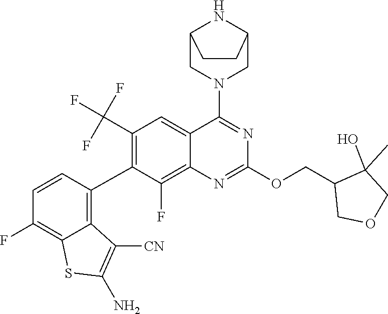 [SMILES: CC1(O)COCC1COc1nc(N2CC3CCC(C2)N3)c2cc(C(F)(F)F)c(-c3ccc(F)c4sc(N)c(C#N)c34)c(F)c2n1]

### Step 1: methyl 4-hydroxytetrahydrofuran-3-carboxylate

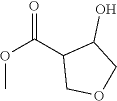

To a solution of methyl 4-oxooxolane-3-carboxylate (10 g, 69.38 mmol) in MeOH (1 L) was added NaBH₄ (5.2 g. 83.26 mmol) in portions at 0° C. The resulting mixture was stirred for 2 h at room temperature under nitrogen atmosphere. The mixture was quenched with saturated NH₄Cl aqueous solution at 0′C and extracted by DCM (5×500 mL). The resulting mixture was concentrated under vacuum. The crude product (5.7 g) was directly used in the next step without further purification.

### Step 2: methyl 4-((tert-butyldimethylsilyl)oxy)tetrahydrofuran-3-carboxylate

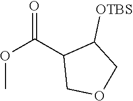 [SMILES: COC(=O)C1COCC1O[Si](C)(C)C(C)(C)C]

A solution of methyl 4-hydroxytetrahydrofuran-3-carboxylate (5.7 g, 39.00 mmol), TBSCl (7.64 g, 50.00 mmol) and imidazole (5.31 g, 78.00 mmol) in DMF (57 mL) was stirred overnight at room temperature under nitrogen atmosphere. The reaction was quenched with water (30 mL) at room temperature. The aqueous layer was extracted with EtOAc (3×30 mL). The combined organic layers were washed with brine and dried over anhydrous Na₂SO₄. After filtration, the filtrate was concentrated under reduced pressure. The residue was purified by silica gel column chromatography and eluted with petroleum ether:EtOAc (5%-10%) to give the title product (9.5 g, 94% yield) as colorless oil.

### Step 3: 4-(hydroxymethyl)tetrahydrofuran-3-ol

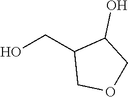

LiAlH₄ (2.4 g, 63.16 mmol) was suspended in THF (48 mL). A solution of methyl 4-((tert-butyldimethylsilyl)oxy)tetrahydrofuran-3-carboxylate (9.5 g, 36.48 mmol) in THF (57 mL) was slowly added dropwise at 0° C. and the mixture was stirred at 0° C. for 1 h. LiAlH₄ (2.4 g, 63.16 mmol) was added, and the mixture was stirred at room temperature for 2.5 h. The mixture was heated at refluxing for 1 h. The reaction solution was cooled to room temperature. Then, water (2.9 ml), 15% aqueous sodium hydroxide solution (2.9 ml) and water (5.8 ml) were added at 0° C. to quench the reaction. The resulting mixture was stirred for 15 min at room temperature and dried over anhydrous MgSO₄. The precipitated insoluble matter was filtered off through Celite. The filtrate was concentrated. The crude product (4.5 g) was directly used in the next step without further purification.

### Step 4: 4-(((tert-butyldimethylsilyl)oxy)methyl)tetrahydrofuran-3-ol

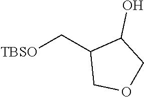 [SMILES: CC(C)(C)[Si](C)(C)OCC1COCC1O]

A solution of 4-(hydroxymethyl)tetrahydrofuran-3-ol (2 g, 16.93 mmol), TBSCl (1.54 g, 10.20 mmol) and imidazole (2.31 g, 33.86 mmol) in DMF (100 mL) was stirred overnight at room temperature. The reaction was quenched with water (50 mL) at room temperature. The aqueous layer was extracted with EtOAc (3×50 mL). The combined organic layers were washed with brine and dried over anhydrous Na₂SO₄. After filtration, the filtrate was concentrated under reduced pressure. The residue was purified by silica gel column chromatography and eluted with petroleum ether:EtOAc (5%-10%) to give the title product (1.7 g, 43% yield) as colorless oil. MS (ESI, m/e) [M+H]⁺ 233.15.

### Step 5: 4-(((tert-butyldimethylsilyl)oxy)methyl)dihydrofuran-3(2H)-one

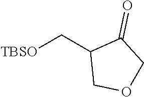 [SMILES: CC(C)(C)[Si](C)(C)OCC1COCC1=O]

A solution of 4-(((tert-butyldimethylsilyl)oxy)methyl)tetrahydrofuran-3-ol (1.7 g, 7.31 mmol) and DMP (6.21 g, 14.63 mmol) in DCM (16 mL) was stirred for 5 h at 40 t. The resulting mixture was filtered, and the filtered cake was washed with DCM (3×10 mL). The filtrate was concentrated under reduced pressure. The residue was purified by silica gel column chromatography and eluted with petroleum ether:EtOAc (5%-20%) to give the title product (1.3 g, 77% yield) as colorless oil. MS (ESI, m/e) [M+H]⁺ 231.15.

### Step 6: 4-(((tert-butyldimethylsilyl)oxy)methyl)-3-methyltetrahydrofuran-3-ol

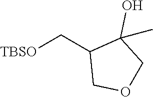 [SMILES: CC1(O)COCC1CO[Si](C)(C)C(C)(C)C]

To a solution of 4-(((tert-butyldimethylsilyl)oxy)methyl)dihydrofuran-3(2H)-one (1.2 g, 5.21 mmol) in THF (10 mL) was added MeMgBr (1.0 M solution, 0.87 mL) at -78° C. The mixture was stirred for 1 h at room temperature and quenched by saturated NH₄Cl aqueous solution. After removal of all volatiles under reduced pressure, the residue was diluted with DCM. The organic layer was washed twice with brine, dried over MgSO₄ and filtered. The filtrate was concentrated to give the residue which was purified by silica gel column chromatography and eluted with petroleum ether:EtOAc (5%-20%) to give the title product (1 g, 78% yield) as colorless oil. MS (ESI, m/e) [M+H]⁺ 247.15.

### Step 7: 4-(hydroxymethyl)-3-methyltetrahydrofuran-3-ol

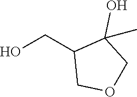

A solution of 4-(((tert-butyldimethylsilyl)oxy)methyl)-3-methyltetrahydro furan-3-ol (500 mg, 2.03 mmol) and TBAF (6 mL, 6.09 mmol, 1 M in THF) in THF (10 mL) was stirred for 2 h at room temperature under nitrogen atmosphere. The resulting mixture was diluted with water (5 mL). The aqueous layer was extracted with EtOAc (5×5 mL). The residue was purified by silica gel column chromatography and eluted with DCM/MeOH (0%-5%) to give the title product (10 mg, 3.73% yield) as colorless oil.

### Step 8: tert-butyl 3-(7-(2-((tert-butoxycarbonyl)amino)-3-cyano-7-fluorobenzo[b]thiophen-4-yl)-8-fluoro-2-((4-hydroxy-4-methyltetrahydrofuran-3-yl)methoxy)-6-(trifluoromethyl)quinazolin-4-yl)-3,8-diazabicyclo[3.2.1]octane-8-carboxylate

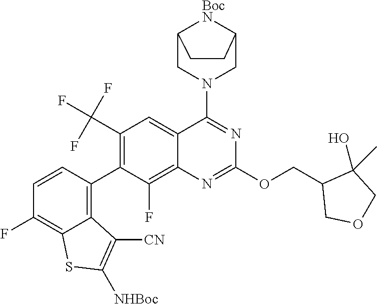 [SMILES: CC(C)(C)OC(=O)Nc1sc2c(F)ccc(-c3c(C(F)(F)F)cc4c(N5CC6CCC(C5)N6C(=O)OC(C)(C)C)nc(OCC5COCC5(C)O)nc4c3F)c2c1C#N]

To a solution of 4-(hydroxymethyl)-3-methyltetrahydrofuran-3-ol (10 mg, 0.08 mmol) and tert-butyl 3-(7-(2-((tert-butoxycarbonyl)amino)-3-cyano-7-fluorobenzo[b]thiophen-4-yl)-2,8-difluoro-6-(trifluoromethyl)quinazolin-4-yl)-3,8-diazabicyclo[3.2.1]octane-8-carboxylate (37.1 mg, 0.051 mmol) in THF (0.5 mL) was added NaH (2.42 mg, 0.101 mmol) in portions at 0° C. under nitrogen atmosphere. The resulting mixture was stirred for 2 h at room temperature under nitrogen atmosphere. The reaction was quenched with saturated NH₄Cl aqueous solution (2 mL) at 0° C. The aqueous layer was extracted with EtOAc (3×10 mL). The resulting mixture was concentrated under reduced pressure. The residue was purified by Prep-TLC (Petroleum ether:EtOAc=1:2) to give the title product (13 mg, 30% yield) as white solid. MS (ESI, m/e) [M+H]⁺ 847.30.

### Step 9: 4-(4-(3,8-diazabicyclo[3.2.1]octan-3-yl)-8-fluoro-2-((4-hydroxy-4-methyltetrahydrofuran-3-yl)methoxy)-6-(trifluoromethyl)quinazolin-7-yl)-2-amino-7-fluorobenzo[b]thiophene-3-carbonitrile

A solution of tert-butyl 3-(7-(2-((tert-butoxycarbonyl)amino)-3-cyano-7-fluorobenzo[b]thiophen-4-yl)-8-fluoro-2-((4-hydroxy-4-methyltetrahydrofuran-3-yl)methoxy)-6-(trifluoromethyl)quinazolin-4-yl)-3,8-diazabicyclo[3.2.1]octane-8-carboxylate (12 mg, 0.014 mmol) and TFA (0.2 mL) in DCM (1 mL) was stirred for 2 h at room temperature under nitrogen atmosphere. The resulting mixture was concentrated under reduced pressure. The residue was purified by Prep-HPLC with the following conditions (Column: XBridge Shield RP18 OBD Column, 30*150 mm, 5 μm; Mobile Phase A: Water (0.05% TFA), Mobile Phase B: ACN; Flow rate: 60 mL/min; Gradient: 22% B to 52% B in 9 min) to give the title product (1 mg, 11% yield, TFA salt). ¹H NMR (300 MHz, CD₃OD) δ 8.11 (s, 1H), 7.24-7.19 (m, 1H), 7.05-6.99 (m, 1H), 4.79-4.60 (m, 3H), 4.54-4.42 (m, 1H), 4.23 (s, 2H), 4.14-4.11 (m, 1H), 3.98-3.80 (m, 3H). 3.79-3.66 (m, 2H), 2.64-2.48 (m, 1H), 2.13 (s, 4H), 1.44 (s, 3H). MS (ESI, m/e) [M+H]⁺ 647.25.

### Example 28: 4-(4-(3,8-diazabicyclo[3.2.1]octan-3-yl)-8-fluoro-2-((3-methoxyoxetan-3-yl)methoxy)-6-(trifluoromethyl)quinazolin-7-yl)-2-amino-7-fluorobenzo[b]thiophene-3-carbonitrile

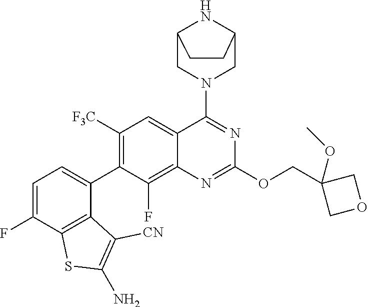 [SMILES: COC1(COc2nc(N3CC4CCC(C3)N4)c3cc(C(F)(F)F)c(-c4ccc(F)c5sc(N)c(C#N)c45)c(F)c3n2)COC1]

Example 28 was prepared by similar procedure as described in Example 3 by replacing (3-methoxytetrahydrofuran-3-yl)methanol with (3-methoxyoxetan-3-yl)methanol to give the title product (2.65 mg). ¹H NMR (500 MHz. CD₃OD) S 8.13 (s, 1H). 7.22-7.20 (m, 1H), 7.02-6.99 (m, 1H), 4.84-4.82 (m, 2H), 4.74-4.72 (m, 2H), 4.64-4.62 (m, 2H), 4.59-4.56 (m, 1H), 4.47-4.45 (m, 1H), 3.73-3.70 (m, 1H), 3.66-3.64 (m, 3H), 3.40 (s, 3H), 1.87-1.77 (m, 4H). MS (EST, m/e) [M+H]⁺ 633.4.

### Example 29: 4-(4-(3,8-diazabicyclo[3.2.1]octan-3-yl)-8-fluoro-2-((1-methyl-2-oxabicyclo[2.1.1]hexan-4-yl)methoxy)-6-(trifluoromethyl)quinazolin-7-yl)naphthalen-2-ol

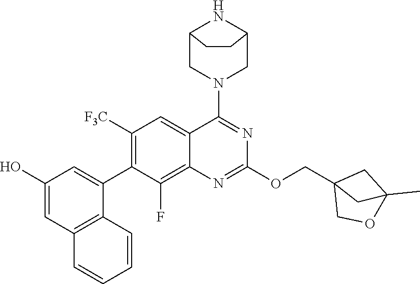 [SMILES: CC12CC(COc3nc(N4CC5CCC(C4)N5)c4cc(C(F)(F)F)c(-c5cc(O)cc6ccccc56)c(F)c4n3)(CO1)C2]

### Step 1: tert-butyl 3-(8-fluoro-7-(3-methoxymethoxy)naphthalen-1-yl)-2-((1-methyl-2-oxabicyclo[2.1.1]hexan-4-yl)methoxy)-6-(trifluoromethyl) quinazolin-4-yl)-3,8-diazabicyclo[3.2.1]octane-8-carboxylate

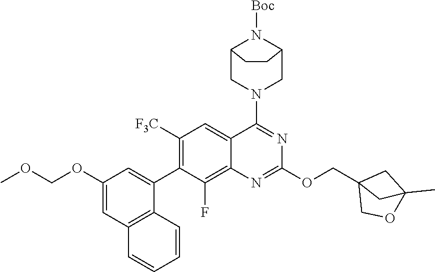

To a mixture of tert-butyl 3-(7-bromo-8-fluoro-2-((1-methyl-2-oxabicyclo [2.1.1]hexan-4-yl)methoxy)-6-(trifluoromethyl)quinazolin-4-yl)-3,8-diaza bicyclo [3.2.1]octane-8-carboxylate (50 mg, 0.08 mmol). 2-(3-(methoxy methoxy) naphthalen-1-yl)-4,4,5,5-tetramethyl-1,3,2-dioxaborolane (49 mg, 0.16 mmol), DPEPhosPdCl₂ (11 mg, 0.016 mmol) and Cs₂CO₃ (77 mg, 0.24 mmol) was added toluene (5 mL). The solution was degassed with N₂ for 3 times. The mixture was stirred for 3 h at 100° C. The cooled resulting mixture was filtered, and the filtrate was concentrated. The residue was purified by chromatography column on silica to give the title product (44 mg). MS (ESI, m/e) [M+H]⁺ 739.5.

### Step 2: 4-(4-(3,8-diazabicyclo[3.2.1]octan-3-yl)-8-fluoro-2-((1-methyl-2-oxabicyclo[2.1.1]hexan-4-yl)methoxy)-6-(trifluoromethyl)quinazolin-7-yl) naphthalen-2-ol

To a solution of tert-butyl 3-(8-fluoro-7-(3-(methoxymethoxy)naphthalen-1-yl)-2-((1-methyl-2-oxabicyclo[2.1.1]hexan-4-yl)methoxy)-6-(trifluoromethyl) quinazolin-4-yl)-3,8-diazabicyclo[3.2.1]octane-8-carboxylate (44 mg, 0.06 mmol) in DCM (10 mL) was added TFA (2 mL). The mixture was stirred at room temperature for 2 to 3 h. The resulting mixture was concentrated to dryness, then diluted with DCM/MeOH=10/1 (5 mL). Next, 5 drops of 7 M NH₃/MeOH solution was added to the mixture. The solution was furtherly concentrated to dryness and purified by chromatography to give the title product (2.64 mg). NMR (500 MHz, CD₃OD) δ8.21 (s, 1H), 7.74-7.72 (m, 1H). 7.41-7.37 (m, 1H), 7.25-7.24 (m, 111). 7.17-7.16 (m, 2H), 7.04-7.02 (m, 1H), 4.75-4.70 (m, 2H), 4.57-4.53 (m, 2H), 3.79 (s, 2H), 3.72-3.69 (m, 4H), 1.87-1.81 (m, 6H), 1.62-1.61 (m, 2H), 1.42 (s, 3H). MS (EST, m/e) [M+H]⁺ 595.5.

### Example 30: 4-(((7-(2-amino-3-cyano-7-fluorobenzo[b]thiophen-4-yl)-4-(3,8-diazabicyclo[3.2.1]octan-3-yl)-8-fluoro-6-(trifluoromethyl)quinazolin-2-yl)oxy) methyl)-2-oxabicyclo[2.1.1]hexane-1-carbonitrile

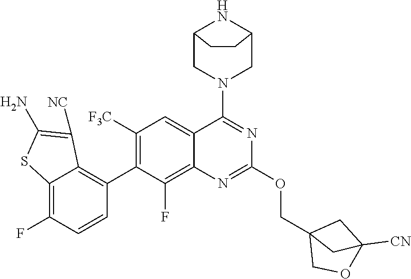 [SMILES: N#Cc1c(N)sc2c(F)ccc(-c3c(C(F)(F)F)cc4c(N5CC6CCC(C5)N6)nc(OCC56COC(C#N)(C5)C6)nc4c3F)c12]

### Step 1: 1-(iodomethyl)-4-(((4-methoxybenzyl)oxy)methyl)-2-oxabicyclo [2.1.1] hexane

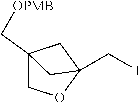

To a solution of (1-(iodomethyl)-2-oxabicyclo[2.1.1]hexan-4-yl)methanol (2.54 g, 10.0 mmol) in THE (20 mL) was added NaH (1.20 g, 30.0 mmol) at room temperature. The resulting mixture was stirred at room temperature for 1 h. Then 1-(chloromethyl)-4-methoxybenzene was added to the reaction mixture and stinted at room temperature for 1 h. After completion, the reaction mixture was quenched with water (30 mL), extracted with EtOAc (100 mL) and washed with saturated NaCl (50 mL×3) aqueous solution. The organic layer was dried over anhydrous Na₂SO₄, filtered and the filtrate was concentrated to give the residue. The residue was purified by silica gel column chromatography and eluted with 0-30% ethyl acetate in petroleum ether to give the title product (1.87 g, 5.0 mmol).

### Step 2: (4-(((4-methoxybenzyl)oxy)methyl)-2-oxabicyclo[2.1.1]hexan-1-yl)methanol

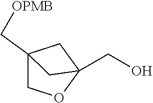

To a solution of 1-(iodomethyl)-4-(((4-methoxybenzyl)oxy)methyl)-2-oxa bicyclo[2.1.1]hexane (1.87 g, 5.0 mmol) in DMSO (30 mL) was added KOAc (2.45 g, 25 mmol) at room temperature. The resulting mixture was stirred at 110° C. for 16 h. After completion, the reaction mixture was diluted with EtOAc (200 mL) and washed with saturated NaCl (50 mL×3) aqueous solution. The organic layer was dried over anhydrous Na₂SO₄, filtered and the filtrate was concentrated to give the residue. The residue was added sodium ethanolate (680 mg, 10 mmol) and EtOH (10 mL). Then the reaction mixture was stirred for 1 h at room temperature. After completion, the reaction mixture was directly concentrated to give the residue. The residue was purified by silica gel column chromatography and eluted with 0-80% ethyl acetate in petroleum ether to give the title product (528.2 mg, 2.0 mmol).

### Step 3: 4-(((4-methoxybenzyl)oxy)methyl)-2-oxabicyclo[2.1.1]hexane-1-carbaldehyde

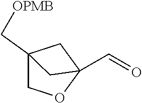

To a solution of (4-(((4-methoxybenzyl)oxy)methyl)-2-oxabicyclo[2.1.1]hexan-1-yl)methanol (528.2 mg, 2.0 mmol) in DCM (10 mL) was added DMP (848 mg, 2.0 mmol) at room temperature. The resulting mixture was stirred at room temperature for 1 h. After completion, the reaction mixture was diluted with EtOAc (100 mL) and washed with saturated NaCl (50 mL×3) aqueous solution. The organic layer was dried over anhydrous Na₂SO₄, filtered and the filtrate was concentrated to give the crude title product (1.0 g crude).

### Step 4: 4-(((4-methoxybenzyl)oxy)methyl)-2-oxabicyclo[2.1.1]hexane-1-carbonitrile

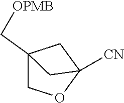

To a solution of 4-(((4-methoxybenzyl)oxy)methyl)-2-oxabicyclo[2.1.1]hexane-1-carbaldehyde (100 mg crude) in pyridine (2 mL) was added hydroxylamine hydrochloride (87.0 mg, 1.26 mmol) at room temperature. The resulting mixture was stirred at room temperature for 16 h. After completion, the reaction mixture was directly concentrated to give the crude product (150 mg crude). Then triethylamine (0.15 ml, 1.1 mmol), methanesulfonyl chloride (0.06, 0.41 mmol) and DCM (5 mL) was added to the crude product. The resulting mixture was stirred at room temperature for 16 h. After completion, the residue was purified by silica gel chromatography and eluted with 0-60% ethyl acetate in petroleum ether to give the title product (60.0 mg, 0.23 mol).

### Step 5: 4-(hydroxymethyl)-2-oxabicyclo[2.1.1]hexane-1-carbonitrile

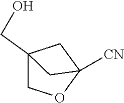

To a solution of 4-(hydroxymethyl)-2-oxabicyclo[2.1.1]hexane-1-carbonitrile (60.0 mg, 0.23 mol) in DCM (5 mL) and water (1 mL) was added DDQ (90.8 mg, 0.4 mmol) at room temperature. The resulting mixture was stirred at room temperature for 2 h. After completion, the reaction mixture was diluted with water (10 mL), extracted with DCM (10 mL) and washed with saturated NaCl (10 mL×3) aqueous solution. The organic layer was dried over anhydrous Na₂SO₄, filtered and the filtrate was concentrated to give the residue. The residue was purified by silica gel chromatography and eluted with 0-60% ethyl acetate in petroleum ether to give the title product (20.0 mg, 0.14 mmol).

Example 30 was prepared by similar procedure as described in Example 1 by replacing (1-(difluoromethyl)-2-oxabicyclo[2.1.1]hexan-4-yl)methanol with 4-(hydroxymethyl)-2-oxabicyclo[2.1.1]hexane-1-carbonitrile to give the title product (4.4 mg). ¹H NMR (500 MHz, CD₃OD) δ 8.12 (s, 1H), 7.22-7.20 (m, 1H), 7.02-6.99 (m, 1H), 4.85-4.55 (m, 3H), 3.97 (s, 2H), 3.85-3.75 (m, 5H), 2.39 (s, 2H), 2.02-1.99 (m, 6H). 8 MS (ESI, m/e) [M+1]⁺ 654.5.

### Example 31: 4-(4-(3,8-diazabicyclo[3.2.1]octan-3-yl)-2-((1-(cyclopropanecarbonyl)-3-methylazetidin-3-yl)methoxy)-8-fluoro-6-(trifluoromethyl)quinazolin-7-yl)-2-amino-7-fluorobenzo[b]thiophene-3-carbonitrile

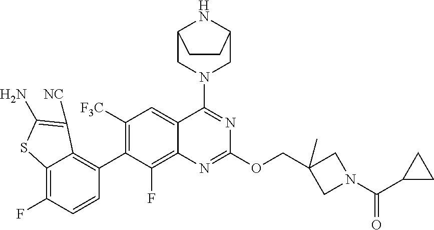 [SMILES: CC1(COc2nc(N3CC4CCC(C3)N4)c3cc(C(F)(F)F)c(-c4ccc(F)c5sc(N)c(C#N)c45)c(F)c3n2)CN(C(=O)C2CC2)C1]

Example 31 was prepared by similar procedure as described in Example 13 by replacing acetyl chloride with cyclopropanecarbonyl chloride to give the title product (4.4 mg). ¹H NMR (500 MHz, CD₃OD) δ 8.12 (s, 1H), 7.23-7.20 (m, 1H), 7.03-7.00 (m, 1H), 4.68-4.47 (m, 4H), 4.39-4.35 (m, 1H), 4.05-3.97 (m, 4H). 3.83-3.69 (m, 3H), 2.00 (s, 4H), 1.61-1.56 (m, 3H), 1.47 (s, 3H), 0.86-0.79 (m, 4H). MS (ESI, m/e) [M+1]⁺ 684.5.

### Example 32: 4-(4-(3,8-diazabicyclo[3.2.1]octan-3-yl)-2-((3-ethyloxetan-3-yl)methoxy)-8-fluoro-6-(trifluoromethyl)quinazolin-7-yl)-2-amino-7-fluorobenzo[b]thiophen-3-carbonitrile

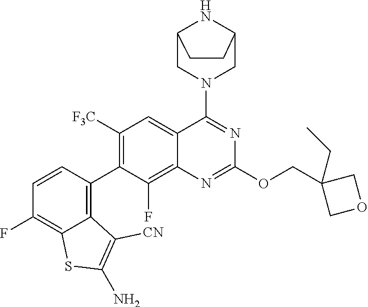 [SMILES: CCC1(COc2nc(N3CC4CCC(C3)N4)c3cc(C(F)(F)F)c(-c4ccc(F)c5sc(N)c(C#N)c45)c(F)c3n2)COC1]

### Step 1: tert-butyl 3-(7-(2-((tert-butoxycarbonyl)amino)-3-cyano-7-fluorobenzo [b]thiophen-4-yl)-2-((3-ethyloxetan-3-yl)methoxy)-8-fluoro-6-(trifluoromethyl) quinazolin-4-yl)-3,8-diazabicyclo[3.2.1]octane-8-carboxylate

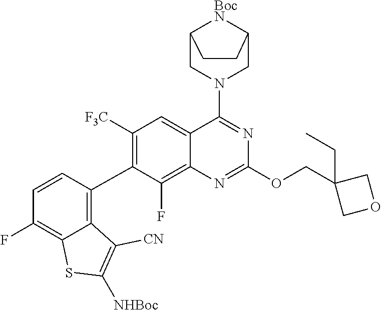 [SMILES: CCC1(COc2nc(N3CC4CCC(C3)N4C(=O)OC(C)(C)C)c3cc(C(F)(F)F)c(-c4ccc(F)c5sc(NC(=O)OC(C)(C)C)c(C#N)c45)c(F)c3n2)COC1]

To a solution of (3-ethyloxetan-3-yl)methanol (12 mg, 0.1 mmol) in THE (2 mL) was added LiHMDS (0.1 mL, 0.1 mmol) at 0° C. The resulting mixture was stirred at room temperature for 0.5 h. Then, tert-butyl 3-(7-(2-((tert-butoxycarbonyl)amino)-3-cyano-7-fluorobenzo[b]thiophen-4-yl)-8-fluoro-2-(methylsulfonyl)-6-(trifluoromethyl)quinazolin-4-yl)-3,8-diazabicyclo[3.2.1]octane-8-carboxylate (40 mg, 0.05 mmol) was added to the reaction mixture and stirred at room temperature for 1 h. After completion, the reaction mixture was diluted with EtOAc (40 mL) and washed with saturated NaCl (15 mL×3) aqueous solution. The organic layer was dried over anhydrous Na₂SO₄, filtered and the filtrate was concentrated to give the residue. The residue was purified by Prep-TLC (Petroleum ether:EtOAc=2:1) to give the title product (35 mg, 84.4%). MS (ESI, m/e) [M+1]⁺ 831.5.

### Step 2: 4-(4-(3,8-diazabicyclo[3.2.1]octan-3-yl)-2-((3-ethyloxetan-3-yl)methoxy)-8-fluoro-6-(trifluoromethyl)quinazolin-7-yl)-2-amino-7-fluorobenzo[b]thiophene-3-carbonitrile

To a solution of tert-butyl 3-(7-(2-((tert-butoxycarbonyl)amino)-3-cyano-7-fluorobenzo[b]thiophen-4-yl)-2-((3-ethyloxetan-3-yl)methoxy)-8-fluoro-6-(trifluoro methyl)quinazolin-4-yl)-3,8-diazabicyclo[3.2.1]octane-8-carboxylate (70 mg, 0.0842 mmol) in DCM (10 mL) was added TFA (1 mL) at room temperature. The resulting mixture was stirred at room temperature for 3 h. After completion, the reaction mixture was concentrated to give the residue. The residue was purified by prep-HPLC to give the title product (7.5 mg. 14.2%). ¹H NMR (500 MHz, CD₃OD) δ 8.12 (s, 1H), 7.23-7.20 (m, 1H), 7.04-6.96 (m, 1H), 4.64-4.60 (m, 4H), 4.60-4.54 (m, 1H), 4.49-4.46 (m, 3H), 3.69-3.59 (m, 4H), 1.93-1.84 (m, 6H), 1.01-0.98 (m, 3H). MS (ESI, m/e) [M+1]+631.5.

### Example 33: 4-(2-((2-oxaspiro[3.3]heptan-6-yl)methoxy)-4-(3,8-diazabicyclo [3.2.1]octan-3-yl)-8-fluoro-6-(trifluoromethyl)quinazolin-7-yl)-2-amino-7-fluoro benzo[b]thiophene-3-carbonitrile

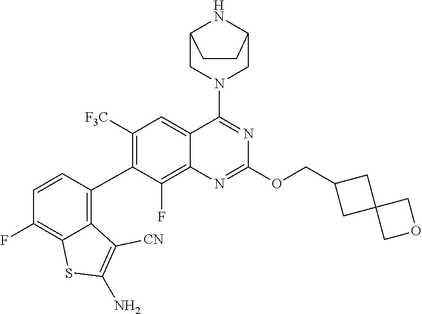 [SMILES: N#Cc1c(N)sc2c(F)ccc(-c3c(C(F)(F)F)cc4c(N5CC6CCC(C5)N6)nc(OCC5CC6(COC6)C5)nc4c3F)c12]

Example 33 was prepared by similar procedure as described in Example 32 by replacing (3-ethyloxetan-3-yl)methanol with (2-oxaspiro[3.3]heptan-6-yl)methanol to give the title product (11.5 mg, 21.7%). ¹H NMR (500 MHz. CD₃OD) δ 8.10 (s, 1H), 7.22-7.19 (m, 1H). 7.02-6.98 (m, 1H), 4.72 (s, 2H), 4.66 (s, 2H), 4.55-4.53 (m, 1H), 4.45-4.42 (m, 1H), 4.37-4.36 (m, 2H), 3.71-3.62 (m, 4H), 2.64-2.58 (m, 1H), 2.45-2.40 (m, 2H), 2.19-2.15 (m, 2H), 1.89-1.76 (m, 4H). MS (ES, m/e) [M+1]643.4.

### Example 34: 4-(4-(3,8-diazabicyclo[3.2.1]octan-3-yl)-2-((3-(1,1-difluoroethyl) oxetan-3-7-yl)-2-amino-7-fluoro benzo[b]thiophene-3-carbonitrile

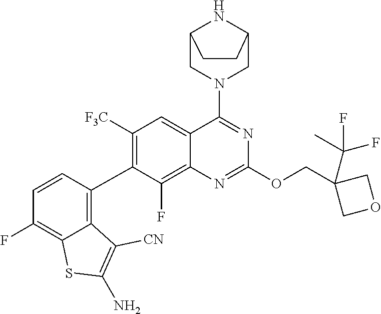 [SMILES: CC(F)(F)C1(COc2nc(N3CC4CCC(C3)N4)c3cc(C(F)(F)F)c(-c4ccc(F)c5sc(N)c(C#N)c45)c(F)c3n2)COC1]

Example 34 was prepared by similar procedure as described in Example 32 by replacing (3-ethyloxetan-3-yl)methanol with (3-(1,1-difluoroethyl)oxetan-3-yl) methanol to give the title product (9.5 mg, 17.7%). ¹H NMR (500 MHz, CD₃OD) δ 8.13 (s, 1H), 7.23-7.20 (m, 1H), 7.03-6.99 (m, 1H), 4.80 (s, 2H), 4.78-4.77 (m, 2H), 4.71-4.70 (m, 2H), 4.59-4.56 (m, 1H), 4.48-4.46 (m, 1H), 3.73-3.65 (m, 4H), 1.83-1.71 (m, 7H). MS (ESI, m/e) [M+1]⁺ 667.4.

### Example 35: 4-(4-(3,8-diazabicyclo[3.2.1]octan-3-yl)-2-((4-(cyanomethyl)tetra hydro-2H-pyran-4-yl)methoxy)-8-fluoro-6-(trifluoromethyl)quinazolin-7-yl)-2-amino -7-fluorobenzo[b]thiophene-3-carbonitrile

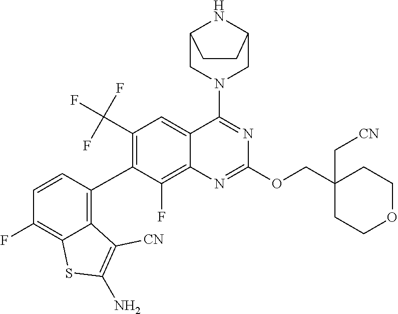 [SMILES: N#CCC1(COc2nc(N3CC4CCC(C3)N4)c3cc(C(F)(F)F)c(-c4ccc(F)c5sc(N)c(C#N)c45)c(F)c3n2)CCOCC1]

Example 35 was prepared by similar procedure as described in Example 37 by replacing methyl tetrahydrofuran-3-carboxylate with ethyl tetrahydro-2H-pyran-4-carboxylate to give the title product (4.5 mg. 19.56% yield). ¹H NMR (300 MHz, CD₃OD) δ 8.13 (s, 1H), 7.26-7.19 (m, 1H), 7.06-6.97 (m, 1H), 4.60-4.46 (m, 4H), 3.87-3.73 (m, 4H), 3.72-3.57 (m, 4H), 2.84 (s, 2H), 1.95-1.76 (m, 6H), 1.75-1.66 (m, 2H). MS (ESI, m/e) [M+H]⁺ 670.30.

### Example 36: 4-(2-((1-acetyl-3-fluoroazetidin-3-yl)methoxy)-4-(−3,8-diaza bicyclo[3.2.1]octan-3-yl)-8-fluoro-6-(trifluoromethyl)quinazolin-7-yl)-2-amino-7-fluorobenzo[b]thiophene-3-carbonitrile

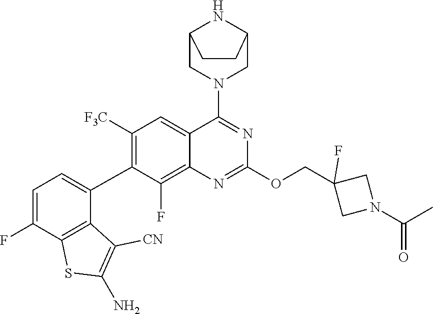 [SMILES: CC(=O)N1CC(F)(COc2nc(N3CC4CCC(C3)N4)c3cc(C(F)(F)F)c(-c4ccc(F)c5sc(N)c(C#N)c45)c(F)c3n2)C1]

### Step 1: (3-fluoroazetidin-3-yl)methanol trifluoroacetate

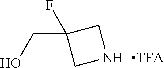

To a solution of tert-butyl 3-fluoro-3-(hydroxymethyl)azetidine-1-carboxylate (648 mg, 33 mmol) in DCM (10 ml) was added TFA (5 ml) at rt. The reaction mixture was stirred at rt for 1h. The solvent was evaporated to dryness and directly used in next step.

Example 36 was prepared by similar procedure as described in Example 8 by replacing (3-methylazetidin-3-yl)methanol hydrochloride with (3-fluoroazetidin-3-yl)methanol trifluoroacetate to give the title product (10 mg, Formate). ¹H NMR (500 MHz, CD₃OD) δ 8.45 (s, 0.5H), 8.13 (s, 1H), 7.23-7.20 (m, 1H), 7.04-7.00 (m, 1H), 4.82-4.81 (m, 2H), 4.71-4.69 (m, 1H), 4.59-4.50 (m, 2H), 4.42-4.38 (m, 1H), 4.29-4.24 (m, 1H), 4.17-4.10 (m, 1H), 4.08-4.02 (m, 2H), 3.87-3.75 (m, 2H), 2.08-2.00 (m, 4H), 1.92 (s, 3H). MS (ESI, mile) [M+1]⁺ 662.5.

### Example 37: 4-(4-(3,8-diazabicyclo[3.2.1]octan-3-yl)-2-((3-(cyanomethyl)tetra hydrofuran-3-yl)methoxy)-8-fluoro-6-(trifluoromethyl)quinazolin-7-yl)-2-amino-7-fluorobenzo[h]thiophene-3-carbonitrile

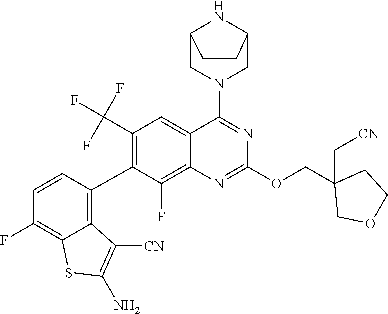 [SMILES: N#CCC1(COc2nc(N3CC4CCC(C3)N4)c3cc(C(F)(F)F)c(-c4ccc(F)c5sc(N)c(C#N)c45)c(F)c3n2)CCOC1]

### Step 1: methyl 3-(cyanomethyl)tetrahydrofuran-3-carboxylate

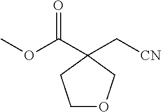

To a solution of LDA (4.23 ml, 8.46 mmol) in THE (8 mL) was added HMPA (5.9 g, 32.92 mmol) at −78° C. and the solution turned red. To the solution was added a solution of methyl tetrahydrofuran-3-carboxylate (1 g, 7.7 mmol) in THE (2 mL) at −78° C. The mixture was stirred for 20 minutes at −78° C. Then 2-bromoacetonitrile (1.85 g, 15.42 mmol) was added and the reaction was warmed to room temperature. The solution was stirred for another 16 hours. The reaction was quenched by saturated NH₄Cl (aq.) (20 mL) at room temperature. The resulting mixture was extracted with EtOAc (100 mL×3). The combined organic layers were washed with brine (100 mL) and dried over anhydrous Na₂SO₄. After filtration, the filtrate was concentrated under reduced pressure. The residue was purified by silica gel column chromatography and eluted with Petroleum ether/EtOAc (100:0-85:15) to give the title product (600 mg. 46% yield).

### Step 2: 2-(3-(hydroxymethyl)tetrahydrofuran-3-1)acetonitrile

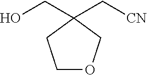

To a stirred mixture of methyl 3-(cyanomethyl)tetrahydrofuran-3-carboxylate (250 mg, 1.48 mmol) in THF (3 mL) was added LiBH₄ (74 mg, 3.40 mmol) in portions at 0° C. The resulting mixture was stirred for additional 3 h at 80° C. The reaction was quenched by saturated NH₄Cl aqueous solution at room temperature. The resulting mixture was extracted with EtOAc (50 mL×3). The combined organic layers were washed with brine (50 mL) and dried over anhydrous Na₂SO₄. After filtration, the filtrate was concentrated under reduced pressure. The residue was purified by silica gel column chromatography and eluted with petroleum ether:EtOAc (100:0-68:32) to give the title product (89 mg, 43% yield).

### Step 3: tert-butyl 3-(7-(2-((tert-butoxycarbonyl)amino)-3-cyano-7-fluorobenzo [b]thiophen-4-yl)-2-((3-(cyanomethyl)tetrahydrofuran-3-yl)methoxy)-8-fluoro-6-trifluoromethyl)quinazolin-4-yl)-3,8-diazabicyclo[3.2.1]octane-8-carboxylate

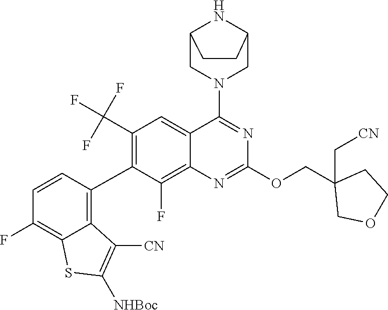 [SMILES: CC(C)(C)OC(=O)Nc1sc2c(F)ccc(-c3c(C(F)(F)F)cc4c(N5CC6CCC(C5)N6)nc(OCC5(CC#N)CCOC5)nc4c3F)c2c1C#N]

To a stirred solution of 2-(3-(hydroxymethyl)tetrahydrofuran-3-yl)acetonitrile (15 mg, 0.11 mmol) and tert-butyl 3-(7-(2-((tert-butoxycarbonyl)amino)-3-cyano-7-fluorobenzo[b]thiophen-4-yl)-2,8-difluoro-6-(trifluoromethyl)quinazolin-4-yl)-3,8-diazabicyclo[3.2.1]octane-8-carboxylate (78.0 mg, 0.11 mmol) in THF (2 mL) was added NaH (5.1 mg, 0.21 mmol, 60%) in portions at 0° C. under nitrogen atmosphere. The resulting mixture was stirred for additional 1 h at room temperature. The reaction was quenched by saturated NH₄Cl aqueous solution (20 mL) at room temperature. The resulting mixture was extracted with EtOAc (40 mL×3). The combined organic layers were washed with brine (10 mL) and dried over anhydrous Na₂SO₄. After filtration, the filtrate was concentrated under reduced pressure. The residue was purified by Prep-TLC (Petroleum ether:EtOAc=1:2) to give the title product (70 mg, 77% yield). MS (ESI, m/e) [M+H]⁺ 856.40.

### Step 4: 4-(4-(3,8-diazabicyclo[3.2.1]octan-3-yl)-2-((3-(cyanomethyl) tetrahydro furan-3-yl)methoxy)-8-fluoro-6-(trifluoromethyl)quinazolin-7-yl)-2-amino-7-fluoro benzo[b]thiophene-3-carbonitrile

Into the 8 mL vial were added tert-butyl 3-(7-(2-(tert-butoxycarbonyl)amino)-3-cyan-7-fluorobenzo [b]thiophen-4-yl)-2-((3-(cyanomethyl)tetrahydrofuran-3-yl)methoxy)-8-fluoro-6-(trifluoromethyl)quinazolin-4-yl)-3,8-diazabicyclo[3.2.1]octane-8-carboxylate (65 mg, 0.076 mmol), DCM (1.5 mL) and TFA (0.5 mL) at room temperature. The resulting mixture was stirred for additional 2 h at room temperature. The resulting mixture was concentrated under reduced pressure. The mixture was basified to pH 9 with saturated NaHCO₃ aqueous solution. The resulting mixture was extracted with EtOAc (3×20 mL). The combined organic layers were washed with brine (10 mL) and dried over anhydrous Na₂SO₄. After filtration, the filtrate was concentrated under reduced pressure. The residue was purified by Prep-HPLC (Column: Xselect CSH C₁₋₈ OBD Column 30*150 mm 5 μm; Mobile Phase A: Water (0.1% formic acid), Mobile Phase B: CH₃CN; Flow rate: 60 mL/min; Gradient: 15% B to 45% B in 8 min) to give the title product (10.2 mg, 20.48% yield, Formate). ¹H NMR (300 MHz, DMSO-d) δ 8.22 (s, 1H), 8.10 (s, 3H), 7.29-7.25 (m, 1H), 7.17-7.11 (m, 1H), 4.44-4.30 (m, 4H), 3.87-3.82 (m, 3H), 3.78-3.72 (m, 1H), 3.69-3.54 (m, 5H), 2.94-2.82 (m, 2H), 2.00-1.93 (m, 1H), 1.93-1.81 (m, 1H), 1.72-1.52 (m, 4H). MS (ESI, m/e) [M+H]⁺ 656.10.

### Example 38: 4-(4-(3,8-diazabicyclo[3.2.1]octan-3-yl)-8-fluoro-2-((3-methyl-1-(methylsulfonyl)azetidin-3-yl)methoxy)-6-(trifluoromethyl)quinazolin-7-yl)-2-amino-7-fluorobenzo[b]thiophene-3-carbonitrile

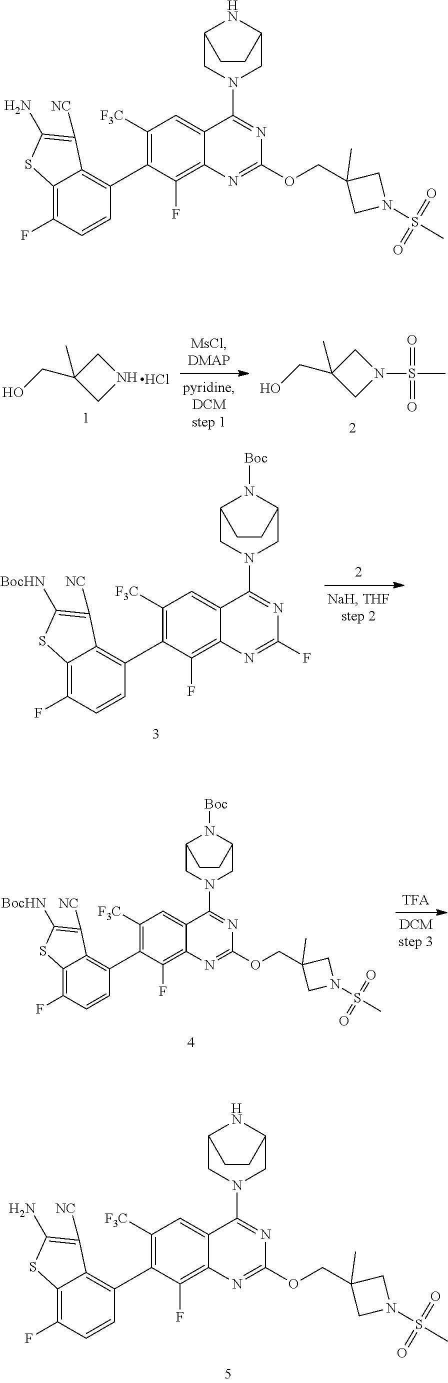

### Step 1: (3-methyl-1-(methylsulfonyl)azetidin-3-yl)methanol

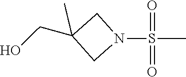

To a solution of (3-methylazetidin-3-yl)methanol hydrochloride (400 mg, 2.91 mmol) in DCM (10 mL) was added dropwise methane sulfonyl chloride (0.23 mL, 2.91 mmol), anhydrous pyridine (0.47 mL, 5.82 mmol) and DMAP (356 mg, 2.91 mmol) at 0° C. Then, the reaction mixture was warmed to rt for 16 h. After completion, the reaction mixture was concentrated to give the crude product. The residue was purified by silica gel column chromatography and eluted with 50% ethyl acetate in petroleum ether to 2% methanol in DCM to give the crude title product (360 mg, 1.0 mmol, purity=50%).

Example 38 was prepared by similar procedure as described in Example 13 by replacing 1-(3-(hydroxymethyl)-3-methylazetidin-1-yl)ethan-1-one with (3-methyl-1-(methylsulfonyl)azetidin-3-yl)methanol to give the title product (34 mg). ¹H NMR (500 MHz, CD₃OD) δ 8.12 (s, 1H), 7.23-7.20 (m, 1H), 7.03-7.00 (m, 1H), 4.66-4.46 (m, 4H), 4.00-3.98 (m, 4H), 3.83-3.75 (m, 2H), 3.68-3.67 (m, 2H), 3.01 (s, 3H), 2.00 (s, 4H), 1.46 (s, 3H). MS (ESI, m/e) [M+1]⁺ 694.5.

### Example 39: 4-(2-((1-acetyl-3-methoxyazetidin-3-yl)methoxy)-4-(3,8-diazabicyclo [3.2.1]octan-3-yl)-8-fluoro-6-(trifluoromethyl)quinazolin-7-yl)-2-amino-7-fluorobenzo[b]thiophene-3-carbonitrile

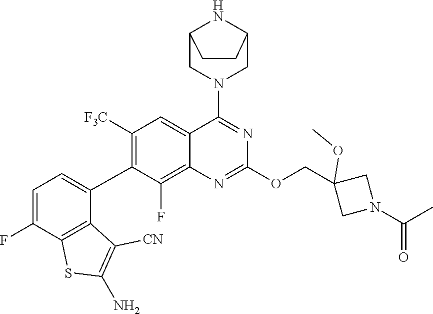 [SMILES: COC1(COc2nc(N3CC4CCC(C3)N4)c3cc(C(F)(F)F)c(-c4ccc(F)c5sc(N)c(C#N)c45)c(F)c3n2)CN(C(C)=O)C1]

Example 39 was prepared by similar procedure as described in Example 3 by replacing (3-methoxytetrahydrofuran-3-yl)methanol with 1-(3-(hydroxy methyl)-3-methoxyazetidin-1-yl)ethan-1-one to give the title product (28.74 mg). ¹H NMR (500 MHz, CD₃OD) δ 8.13 (s, 1H), 7.22-7.20 (m, 1H), 7.03-6.99 (m, 1H), 4.78-4.71 (m, 2H), 4.59-4.56 (m, 1H), 4.49-4.47 (m, 1H), 4.31-4.22 (m, 2H), 4.04-3.97 (m, 2H), 3.73-3.65 (m, 4H), 3.40 (s, 3H), 1.90 (s, 3H), 1.90-1.78 (m, 4H). MS (EST, m/e) [M+H]⁺ 674.4.

### Example 40: 4-(2-((2-acetyl-2-azabicyclo[2.1.1]hexan-4-yl)methoxy)-4-(3,8-diazabicyclo[3.2.1]octan-3-yl)-8-fluoro-6-(trifluoromethyl)quinazolin-7-yl)-2-amino-7-fluorobenzo[b]thiophene-3-carbonitrile

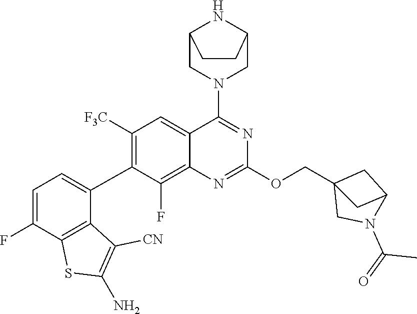 [SMILES: CC(=O)N1CC2(COc3nc(N4CC5CCC(C4)N5)c4cc(C(F)(F)F)c(-c5ccc(F)c6sc(N)c(C#N)c56)c(F)c4n3)CC1C2]

Example 40 was prepared by similar procedure as described in Example 3 by replacing (3-methoxytetrahydrofuran-3-yl)methanol with 1-(4-(hydroxy methyl)-2-azabicyclo[2.1.1]hexan-2-yl)ethan-1-one to give the title product (19.66 mg). ¹H NMR (500 MHz, CD₃OD) δ 8.12 (s, 1H), 7.22-7.20 (m, 1H), 7.03-6.99 (m, 1H), 4.76-4.68 (m, 3H), 4.59-4.48 (m, 2H), 3.75-3.66 (m, 4H), 3.59 (s, 1H), 3.43 (s, 1H), 2.13-2.04 (m, 5H), 1.92-1.87 (m, 4H), 1.64-1.57 (m, 2H). MS (EST, m/e) [M+H]⁺ 671.0.

### Example 41: 4-(2-((2-oxabicyclo[2.1.1]hexan-4-yl)methoxy)-8-fluoro-4-((1S,4R)-2-((2-methoxyethyl)amino)-7-azabicyclo[2.2.1]heptan-7-yl)-6-(trifluoromethyl) quinazolin-7-yl)-2-amino-7-fluorobenzo[b]thiophene-3-carbonitrile

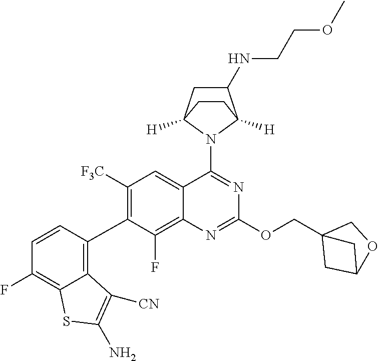 [SMILES: COCCNC1C[C@H]2CC[C@@H]1N2c1nc(OCC23COC(C2)C3)nc2c(F)c(-c3ccc(F)c4sc(N)c(C#N)c34)c(C(F)(F)F)cc12]

### Step 1: tert-butyl (1S,4R)-2-((2-methoxyethyl)amino)-7-azabicyclo[2.2.1]heptane-7-carboxylate

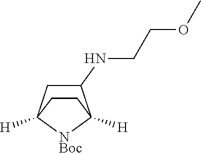 [SMILES: COCCNC1C[C@H]2CC[C@@H]1N2C(=O)OC(C)(C)C]

The solution of tert-butyl (1S,4R)-2-oxo-7-azabicyclo[2.2.1]heptane-7-carboxylate (2.11 g, 10 mmol) and 2-methoxyethan-1-amine (750 mg, 10 mmol) in methanol (40 mL) was stirred at 50° C. for 15 h. Then, NaBH₄ (1520 mg, 40 mmol) was added at 0° C. and the reaction mixture was stinted at 25° C. for 3 h. After completion, the reaction mixture was diluted with EtOAc (50 mL) and washed with saturated NaCl (15 mL×3) aqueous solution. The organic layer was dried over anhydrous Na₂SO₄, filtered and the filtrate was concentrated to give the residue. The residue was purified by silica gel (Petroleum ether:EtOAc=1:1) to give the title product (2.3 g, 85.2%). MS (ESI, m/e) [M+1]+271.5.

### Step 2: tert-butyl (1S,4R)-2-(((benzyloxy)carbonyl)(2-methoxyethyl)amino)-7-azabicyclo[2.2.1]heptane-7-carboxylate

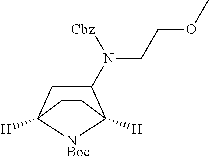 [SMILES: COCCN(C(=O)OCc1ccccc1)C1C[C@H]2CC[C@@H]1N2C(=O)OC(C)(C)C]

The mixture of tert-butyl (1S,4R)-2-((2-methoxyethyl)amino)-7-azabicyclo[2.2.1]heptane-7-carboxylate (1.68 g. 6.0 mmol). CbzCl (1.02 g, 6.0 mmol) and DIPEA (1.55 g, 12.0 mmol) in DCM (20 mL) was stirred at 25° C. for 3 h. After completion, the reaction mixture was diluted with EtOAc (40 mL) and washed with saturated NaCl (15 mL×3) aqueous solution. The organic layer was dried over anhydrous Na₂SO₄, filtered and the filtrate was concentrated to give the residue. The residue was purified by silica gel (Petroleum ether:EtOAc=5:1) to give the title product (1.6 g, 66.1%). MS (ESI, m/e) [M+1]⁺405.5.

### Step 3: benzyl ((1S,4R)-7-azabicyclo[2.2.1]heptan-2-yl)(2-methoxyethyl)carbamate hydrogen chloride salt

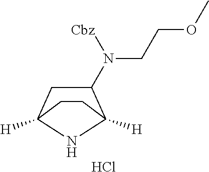 [SMILES: COCCN(C(=O)OCc1ccccc1)C1C[C@H]2CC[C@@H]1N2 ; Cl]

The solution of tert-butyl (1S,4R)-2-(((benzyloxy)carbonyl)(2-methoxyethyl)amino)-7-azabicyclo[2.2.1]heptane-7-carboxylate (606 mg, 1.5 mmol) in HCl/dioxane solution (4N, 10 mL) was stirred at room temperature for 4 h. After completion, the reaction mixture was concentrated. The residue was washed with ethyl ether and directly used in next step without further purification. MS (ESI, m/e) [M+1]+305.4.

### Step 4: benzyl ((1S,4R)-7-(7-bromo-2-chloro-8-fluoro-6-(trifluoromethyl) quinazolin-4-yl)-7-azabicyclo[2.2.1]heptan-2-yl)(2-methoxyethyl)carbamate

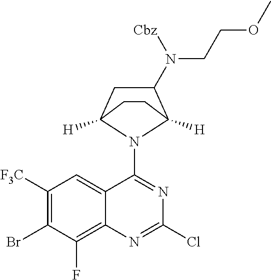 [SMILES: COCCN(C(=O)OCc1ccccc1)C1C[C@H]2CC[C@@H]1N2c1nc(Cl)nc2c(F)c(Br)c(C(F)(F)F)cc12]

The mixture of 7-bromo-2,4-dichloro-8-fluoro-6-(trifluoromethyl) quinazoline (364 mg, 1.0 mmol), benzyl ((1S,4R)-7-azabicyclo[2.2.1]heptan-2-yl)(2-methoxyethyl)carbamate hydrogen chloride salt (340 mg, 1.0 mmol) and DIPEA (516 mg, 4.0 mmol) in DCM (10 mL) was stirred at 25° C. for 1 h under N₂ atmosphere. After completion, the reaction mixture was diluted with DCM (50 mL) and washed with water (15 mL×3). The organic layer was dried over anhydrous Na₂SO₄, filtered and the filtrate was concentrated to give the residue. The residue was purified by silica gel (Petroleum ether:EtOAc=3:1) to give the title product (444 mg, 70.5%). MS (ESI, m/e) [M+1]+631.4.

### Step 5: benzyl ((1S,4R)-7-(2-((2-oxabicyclo[2.1.1]hexan-4-yl)methoxy)-7-bromo -8-fluoro-6-(trifluoromethyl)quinazolin-4-yl)-7-azabicyclo[2.2.1]heptan-2-yl)(2-methoxyethyl)carbamate

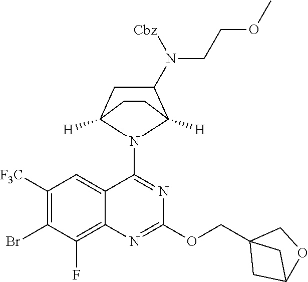 [SMILES: COCCN(C(=O)OCc1ccccc1)C1C[C@H]2CC[C@@H]1N2c1nc(OCC23COC(C2)C3)nc2c(F)c(Br)c(C(F)(F)F)cc12]

The solution of benzyl ((1S,4R)-7-(7-bromo-2-chloro-8-fluoro-6-(trifluoro methyl)quinazolin-4-yl)-7-azabicyclo[2.2.1]heptan-2-yl)(2-methoxyethyl)carbamate (444 mg, 0.704 mmol), (2-oxabicyclo[2.1.1]hexan-4-yl)methanol (160 mg, 1.4 mmol) and KF (204 mg, 3.5 mmol) in DMSO (15 mL) was stirred at 100° C. for 4 h. After completion, the reaction mixture was diluted with EtOAc (50 mL) and washed with water (15 mL×3). The organic layer was dried over anhydrous Na₂SO₄, filtered and the filtrate was concentrated to give the residue. The residue was purified by silica gel (Petroleum ether:EtOAc=3:1) to give the title product (290 mg, 58.0%). MS (ESI, m/e) [M+1]⁺ 709.5, 711.5.

### Step 6: benzyl ((1S,4R)-7-(2-((2-oxabicyclo[2.1.1]hexan-4-yl)methoxy)-7-(2-((tert-butoxycarbonyl)amino)-3-cyano-7-fluorobenzo[b]thiophen-4-yl)-8-fluoro-6-(trifluoromethyl)quinazolin-4-yl)-7-azabicyclo[2.2.1]heptan-2-yl)(2-methoylethyl)carbamate

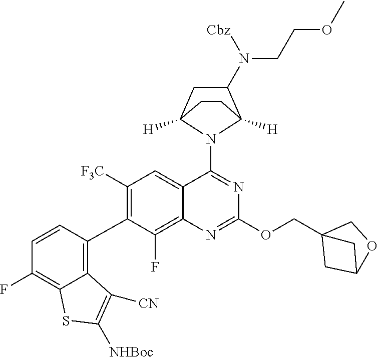 [SMILES: COCCN(C(=O)OCc1ccccc1)C1C[C@H]2CC[C@@H]1N2c1nc(OCC23COC(C2)C3)nc2c(F)c(-c3ccc(F)c4sc(NC(=O)OC(C)(C)C)c(C#N)c34)c(C(F)(F)F)cc12]

The mixture of benzyl ((1S,4R)-7-(2-((2-oxabicyclo[2.1.1]hexan-4-yl)methoxy)-7-bromo-8-fluoro-6-(trifluoromethyl)quinazolin-4-yl)-7-azabicyclo[2.2.1]heptan-2-yl)(2-methoxyethyl)carbamate (213 mg, 0.3 mmol), tert-butyl (3-cyano-7-fluoro-4-(4,4,5,5-tetramethyl-1,3,2-dioxaborolan-2-yl)benzo[b]thiophen-2-yl)carbamate (502 mg, 1.2 mmol), DPEphosPdCl₂ (107 mg, 0.15 mmol) and Cs₂CO₃ (293 mg, 0.9 mmol) in toluene (50 mL) was stirred at 80° C. for 4 h. After completion, the reaction mixture was concentrated and purified by silica gel (Petroleum ether:EtOAc=3:1) to give the titled product (110 mg, 40%). MS (ESI, m/e) [M+1]⁺ 921.5.

### Step 7: tert-butyl (4-(2-((2-oxabicyclo[2.1.1]hexan-4-yl)methoxy)-8-fluoro-4-((1S,4R)-2-((2-methoxyethyl)amino)-7-azabicyclo[2.2.1]heptan-7-yl)-6-(trifluoro methyl)quinazolin-7-yl)-3-cyano-7-fluorobenzo[b]thiophen-2-yl)carbamate

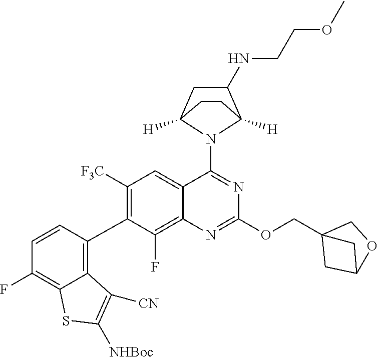 [SMILES: COCCNC1C[C@H]2CC[C@@H]1N2c1nc(OCC23COC(C2)C3)nc2c(F)c(-c3ccc(F)c4sc(NC(=O)OC(C)(C)C)c(C#N)c34)c(C(F)(F)F)cc12]

The mixture of benzyl ((1S,4R)-7-(2-((2-oxabicyclo[2.1.1]hexan-4-yl)methoxy)-7-(2-((tert-butoxycarbonyl)amino)-3-cyano-7-fluorobenzo[b]thiophen-4-yl)-8-fluoro-6-(trifluoromethyl)quinazol in-4-yl)-7-azabicyclo[2.2.1]heptan-2-yl)(2-methoxyethyl)carbamate (100 mg, 0.109 mmol), and 10% Pd/C (100 mg) in methanol (10 mL) was stirred at 20° C. for 4 h under H₂ atmosphere. After completion, the reaction mixture was filtered and directly used in next step without more purification. MS (EST, m/e) [M+1]+787.5.

### Step 8: 4-(2-((2-oxabicyclo[2.1.1]hexan-4-yl)methoxy)-8-fluoro-4-((1S,4R)-2-((2-methoxyethyl)amino)-7-azabicyclo[2.2.1]heptan-7-yl)-6-(trifluoromethyl)quinazolin-7-yl)-2-amino-7-fluorobenzo[b]thiophene-3-carbonitrile

To a solution of tert-butyl (4-(2-((2-oxabicyclo[2.1.1]hexan-4-yl)methoxy)-8-fluoro-4-((1S,4R)-2-((2-methoxyethyl)amino)-7-azabicyclo [2.2.1]heptan-7-yl)-6-(trifluoromethyl)quinazolin-7-yl)-3-cyano-7-fluorobenzo [h]thiophen-2-yl)carbamate (70 mg, 0.089 mmol) in DCM (4 mL) was added TFA (4 mL) at room temperature. The resulting mixture was stirred at room temperature for 3 h. After completion, the reaction mixture was concentrated to give the residue. The residue was purified by prep-HPLC to give the title product (2.0 mg, 3.3%). ¹H NMR (500 MHz, CD₃OD) δ 8.28 (s, 1H), 7.24-7.20 (m, 1H), 7.04-7.00 (m, 1H), 5.10-5.06 (m, 1H), 5.03-4.96 (m, 1H), 4.83-4.79 (m, 2H), 4.57 (s, 2H), 3.73 (s, 3H), 3.65-3.62 (m, 2H), 3.44-3.40 (m, 4H), 2.25-1.86 (m, 6H), 1.70-1.53 (m, 4H). MS (EST, m/e) [M+1]⁺ 687.4.

### Example 42: 4-(4-(3,8-diazabicyclo[3.2.1]octan-3-yl)-8-fluoro-2-((1-methyl-2-oxabicyclo[2.1.1]octane-8-carboxylate

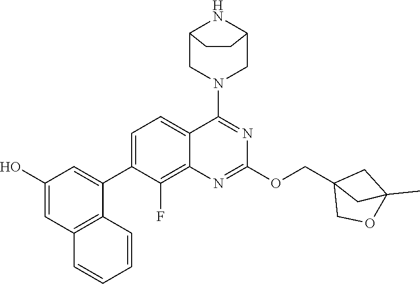 [SMILES: CC12CC(COc3nc(N4CC5CCC(C4)N5)c4ccc(-c5cc(O)cc6ccccc56)c(F)c4n3)(CO1)C2]

### Step 1: tert-butyl 3-(7-bromo-8-fluoro-2-((1-methyl-2-oxabicyclo[2.1.1]hexan-4-yl)methoxy)quinazolin-4-yl)-3,8-diazabicyclo[3.2.1]octane-8-carboxylate

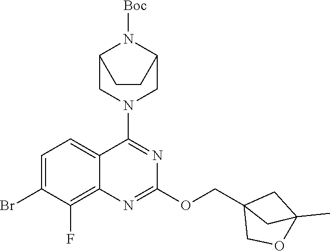 [SMILES: CC(C)(C)OC(=O)N1C2CCC1CN(c1nc(OCC34COC(C)(C3)C4)nc3c(F)c(Br)ccc13)C2]

To a solution of (1-methyl-2-oxabicyclo[2.1.1]hexan-4-yl)methanol (85 mg, 0.66 mmol) in THE (5 mL) was added sodium hydride (27 mg, 0.66 mmol) at 0° C. The reaction mixture was stirred at 0° C. for 0.5 h. Then, tert-butyl 3-(7-bromo-2,8-difluoroquinazolin-4-yl)-3,8-diazabicyclo[3.2.1]octane-8-carboxylate (200 mg, 0.44 mmol) was added to the reaction mixture and stirred at room temperature for 2 h. After completion, the reaction mixture was quenched with water (10 mL), extracted with EtOAc (30 mL×3) and washed with saturated NaCl aqueous solution. The organic layer was dried over anhydrous Na₂SO₄, filtered and the filtrate was concentrated to give the crude title product (240 mg, 0.43 mmol). MS (ESI, m/e) [M+1]+563.1.

### Step 2: tert-butyl 3-(8-fluoro-7-(3-(methoxymethoxy)naphthalen-1-yl)-2-((1-methyl-2-oxabicyclo[2.1.1]hexan-4-yl)methoxy)quinazolin-4-yl)-3,8-diazabicyclo[3.2.1]octane-8-carboxylate

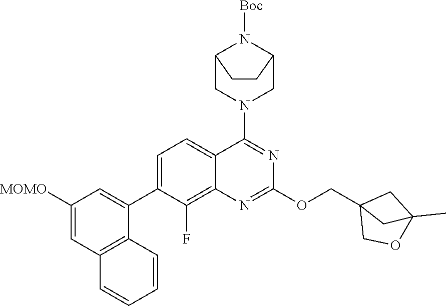

To a solution of tert-butyl 3-(7-bromo-8-fluoro-2-((1-methyl-2-oxabicyclo [2.1.1]hexan-4-yl)methoxy)quinazolin-4-yl)-3,8-diazabicyclo[3.2.1]octane-8-carboxylate (100 mg, 0.18 mmol), 2-(3-(methoxymethoxy)naphthalen-1-yl)-4,4,5,5-tetramethyl-1,3,2-dioxaborolane (110 mg, 0.36 mmol) and PdCl₂(dtbpf) (46 mg, 0.07 mmol) in 1,4-dioxane (3 mL) and H₂O (0.6 mL) was added K₃PO₄ (115 mg, 0.54 mmol) at room temperature. Then, the reaction mixture was stirred at 90° C. for 2 h. After completion, the reaction mixture was quenched with water (30 mL), extracted with EtOAc (30 mL×3) and washed with saturated NaCl aqueous solution. The organic layer was dried over anhydrous Na₂SO₄, filtered and the filtrate was concentrated to give the residue. The residue was purified by silica gel column chromatography and eluted with 0% to 60% ethyl acetate in petroleum ether to give the title product (90 mg, 0.13 mmol). MS (ESI, m/e) [M+1]⁺ 671.3.

### Step 3: 4-(4-(3,8-diazabicyclo[3.2.1]octan-3-yl)-8-fluoro-2-((1-methyl-2-oxa bicyclo[2.1.1]hexan-4-yl)methoxy)quinazolin-7-yl)naphthalen-2-ol

To a solution of tert-butyl 3-(8-fluoro-7-(3-(methoxymethoxy)naphthalen-1-yl)-2-((1-methyl-2-oxabicyclo[2.1.1]hexan-4-yl)methoxy)quinazolin-4-yl)-3,8-diaza bicyclo[3.2.1]octane-8-carboxylate (70 mg, 0.10 mmol) in DCM (24 mL) was added TFA (4.5 mL) at 0′C. The resulting mixture was stirred at room temperature for 2 h. After completion, the reaction mixture was concentrated to give the residue. It was dissolved into 3 mL DCM and adjusted to PH=−10 using 7 N NH₃ in MeOH. After concentration, the residue was purified by pre-HPLC to give the title product (3 mg, Formate). ¹H NMR (500 MHz, CD₃OD) δ 8.50 (s, 0.5H), 7.87-7.73 (m, 2H), 7.46-7.38 (m, 2H), 7.36-7.28 (m, 1H), 7.26-7.17 (m, 2H), 7.09 (s, 1H), 4.74 (s, 2H), 4.63-4.55 (m, 2H), 3.96 (s, 2H), 3.80 (s, 2H), 3.70-3.67 (m, 2H), 2.06-2.04 (m, 4H), 1.82-1.80 (m, 2H), 1.65-1.62 (m, 2H), 1.43 (s, 3H). MS (ESI, m/e) [M+1]+527.4.

### Example 43: 4-(4-(3,8-diazabicyclo[3.2.1]octan-3-yl)-2-((4-(difluoromethyl) tetrahydro-2H-pyran-4-yl)methoxy)-8-fluoro-6-(trifluoromethyl) quinazolin-7-yl)-2-amino-7-fluorobenzo[h]thiophene-3-carbonitrile

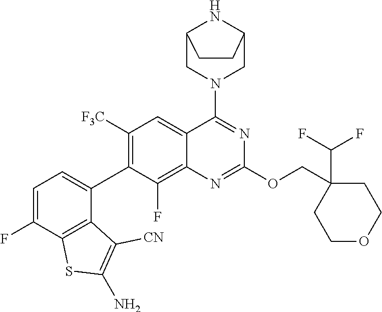 [SMILES: N#Cc1c(N)sc2c(F)ccc(-c3c(C(F)(F)F)cc4c(N5CC6CCC(C5)N6)nc(OCC5(C(F)F)CCOCC5)nc4c3F)c12]

Example 43 was prepared by similar procedure as described in Example 6 by replacing 3-(hydroxymethyl)tetra hydrofuran-3-ol with (4-(difluoromethyl) tetrahydro -2H-pyran-4-yl)methanol to give the title product (12 mg, 26.7% yield). ¹H NMR (500 MHz, CD₃OD) δ 8.12 (s, 1H), 7.25-7.21 (m, 1H), 7.04-7.01 (m, 1H), 6.08-5.86 (m, 1H), 4.85-4.62 (m, 4H), 4.20-4.12 (m, 2H), 3.90-3.69 (m, 6H), 2.14-2.03 (m, 4H), 1.90-1.85 (m, 2H), 1.72-1.69 (m, 2H). MS (ESI, m/e) [M+1]+681.6.

### Example 44: 4-(4-(3,8-diazabicyclo[3.2.1]octan-3-yl)-8-fluoro-2-((3-fluorooxetan-3-yl)methoxy)-6-(trifluoromethyl)quinazolin-7-yl)-2-amino-7-fluorobenzo [b]thiophene-3-carbonitrile

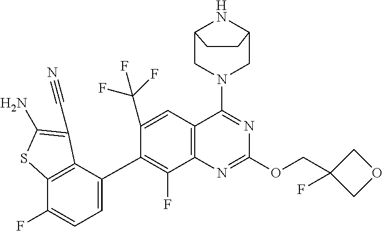 [SMILES: N#Cc1c(N)sc2c(F)ccc(-c3c(C(F)(F)F)cc4c(N5CC6CCC(C5)N6)nc(OCC5(F)COC5)nc4c3F)c12]

### Step 1: tert-butyl 3-(7-(2-((tert-butoxycarbonyl)amino)-3-cyano-7-fluorobenzo [b]thiophen-4-yl)-8-fluoro-2-((3-fluorooxetan-3-yl)methoxy)-6-(trifluoromethyl) quinazolin-4-yl)-3,8-diazabicyclo[3.2.1]octane-8-carboxylate

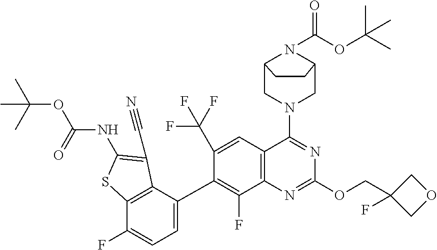 [SMILES: CC(C)(C)OC(=O)Nc1sc2c(F)ccc(-c3c(C(F)(F)F)cc4c(N5CC6CCC(C5)N6C(=O)OC(C)(C)C)nc(OCC5(F)COC5)nc4c3F)c2c1C#N]

To a 20 mL sealed tube was added (3-fluorooxetan-3-yl)methanol (70 mg, 0.660 mmol), tert-butyl 3-(7-(2-((tert-butoxycarbonyl)amino)-3-cyano-7-fluorobenzo[h] thiophen-4-yl)-2,8-difluoro-6-(trifluoromethyl)quinazolin-4-yl)-3,8-diazabicyclo [3.2.1]octane-8-carboxylate (50 mg, 0.068 mmol), THE (6 mL) and NaH (8 mg, 0.2 mmol, 60%). The mixture was stirred at rt for 16 h. The reaction was quenched by saturated NH₄Cl aqueous solution (20 mL) and extracted by EtOAc (40 mL×3). The combined organic phase was dried by anhydrous Na₂SO₄ and concentrated under vacuum to give the crude title product (40 mg, 89.6% yield). MS (ESI, m/e) [M+H]⁺ 821.5.

### Step 2: 4-(4-(3,8-diazabicyclo[3.2.1]octan-3-yl)-8-fluoro-2-((3-fluorooxetan-3-yl)methoxy)-6-(trifluoromethyl)quinazolin-7-yl)-2-amino-7-fluorobenzo[b]thiophene-3-carbonitrile

To a 25 mL round bottom flask was added ten-butyl 3-(7-(2-((tert-butoxy carbonyl)amino)-3-cyano-7-fluorobenzo[b]thiophen-4-yl)-8-fluoro-2-((3-fluorooxetan-3-yl)methoxy)-6-(trifluoromethyl)quinazolin-4-yl)-3,8-diazabicyclo[3.2.1] octane-8-carboxylate (40 mg, 0.049 mmol), TFA (4 mL) and DCM (2 mL). The reaction was stirred at rt for 16 h. The reaction was concentrated under vacuum. The residue was dissolved in EtOAc (40 mL) and saturated Na₂CO₃ aqueous solution (20 mL) was added. The solution was stirred at rt for 10 min and extracted by EtOAc (40 mL×2). The combined organic phase was washed with brine (20 mL), dried over anhydrous Na₂SO₄, and then concentrated under vacuum. The residue was purified by Prep-HPLC (Column: XBridge Shield RP18 OBD Column; Mobile Phase A: Water (10 mmol IL NH₄HCO₃+0.1% NH₃·H₂O), Mobile Phase B: CH₃CN; Gradient: 20% B to 75% B in 9 min) to give the title product (7 mg, 23.0% yield). ¹H NMR (500 MHz, CD₃OD) δ 8.13 (s, 1H). 7.25-7.19 (m, 1H), 7.03-7.00 (m, 1H), 4.78-4.58 (m, 7H), 3.99 (s, 2H), 3.86-3.77 (m, 3H), 2.00 (s, 4H). MS (ESI, m/e) [M+H]⁺ 621.3.

### Example 45: 4-(4-(3,8-diazabicyclo[3.2.1]octan-3-yl)-8-fluoro-2-((1-methoxy cyclopropyl)methoxy)-6-(trifluoromethyl)quinazolin-7-yl)-2-amino-7-fluorobenzo[b] thiophene-3-carbonitrile

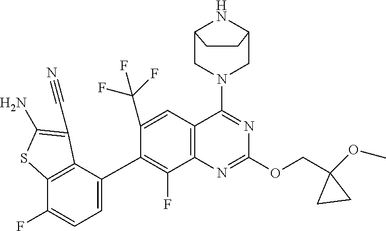 [SMILES: COC1(COc2nc(N3CC4CCC(C3)N4)c3cc(C(F)(F)F)c(-c4ccc(F)c5sc(N)c(C#N)c45)c(F)c3n2)CC1]

Example 45 was prepared by similar procedure as described in Example 44 by replacing (3-fluorooxetan-3-yl)methanol with (1-methoxycyclopropyl)methanol to give the title product (11.8 mg, 39.0% yield). ¹H NMR (500 MHz, CD₃OD) δ 8.11 (s, 1H), 7.23-7.22 (m, 1H), 7.04-7.00 (m, 1H), 4.68-4.59 (m, 4H), 4.23 (s, 2H), 3.92-3.83 (m, 2H), 3.41 (s, 3H), 2.14-2.13 (m, 4H), 0.92 (s, 2H), 0.80 (s. 2H). MS (ESI, m/e) [M+H]⁺ 617.4.

### Example 46: 4-(4-(3,8-diazabicyclo[3.2.1]octan-3-yl)-8-fluoro-2-((3-methyl-1-(oxazol-2-yl)azetidin-3-yl)methoxy)-6-(trifluoromethyl)quinazolin-7-yl)-2-amino-7-fluorobenzo[b]thiophene-3-carbonitrile

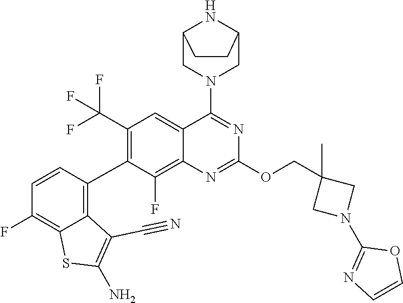 [SMILES: CC1(COc2nc(N3CC4CCC(C3)N4)c3cc(C(F)(F)F)c(-c4ccc(F)c5sc(N)c(C#N)c45)c(F)c3n2)CN(c2ncco2)C1]

Example 46 was prepared by similar procedure as described in Example 24 by replacing azetidin-3-ylmethanol hydrochloride and 2-iodo-1-methyl-1H-imidazole with (3-methylazetidin-3-yl)methanol hydrochloride and 2-iodooxazole to give the title product (12 mg, 26.7% yield). ¹H NMR (500 MHz, CD₃OD) δ 8.13 (s, 1H), 7.53 (s, 1H), 7.22-7.19 (m, 1H), 7.11-7.08 (m, 1H), 7.04-7.01 (m, 1H). 4.77-4.74 (m, 1H), 4.67-4.64 (m, 1H), 4.59-4.54 (m, 2H), 4.30-4.24 (m, 4H), 4.01-3.99 (m, 2H), 3.91-3.80 (m, 2H), 2.20-2.07 (m, 4H), 1.51 (s, 3H). MS (ESI, m/e) [M+1]⁺ 683.6.

### Example 47: 4-(4-(3,8-diazabicyclo[3.2.1]octan-3-yl)-2-((1-(cyclopropoxy methyl)-2-oxabicyclo[2.1.1]hexan-4-yl)methoxy)-8-fluoro-6-(trifluoromethyl) quinazolin-7-yl)-2-amino-7-fluorobenzo[b]thiophene-3-carbonitrile

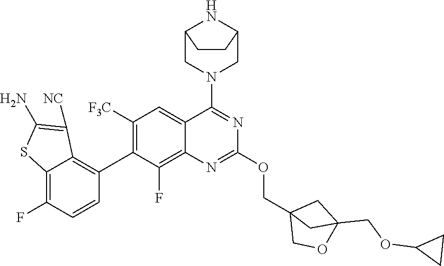 [SMILES: N#Cc1c(N)sc2c(F)ccc(-c3c(C(F)(F)F)cc4c(N5CC6CCC(C5)N6)nc(OCC56COC(COC7CC7)(C5)C6)nc4c3F)c12]

Example 47 was prepared by similar procedure as described in Example 11 by replacing sodium methanolate with sodium cyclopropanolate to give the title product (1 mg, 16.1% yield). ¹H NMR (500 MHz, CD₃OD) δ 8.12 (s, 1H), 7.28-7.13 (m, 1H), 7.02-6.99 (m, 1H), 4.81-4.69 (m, 2H), 4.63-4.54 (m, 2H), 3.94-3.88 (m, 2H), 3.82-3.80 (m, 2H), 3.79-3.76 (m, 1H), 3.75-3.73 (m, 2H), 3.44-3.39 (m, 2H), 2.03-1.92 (m, 4H), 1.91-1.84 (m, 2H), 1.72-1.60 (m, 2H), 0.60-0.50 (m, 2H), 0.50-0.41 (m, 2H). MS (ESI, m/e) [M+1]⁺ 699.6.

### Example 48: 4-(4-(3,8-diazabicyclo[3.2.1]octan-3-yl)-2-((3-(difluoromethyl) oxetan-3-yl)methoxy)-8-fluoro-6-(trifluoromethyl)quinazolin-7-yl)-2-amino-7-fluoro benzo[b]thiophene-3-carbonitrile

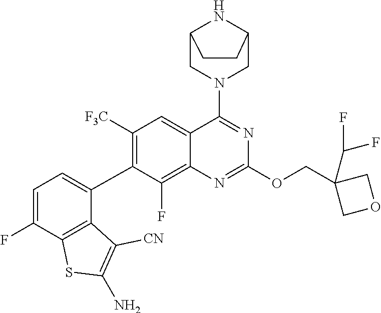 [SMILES: N#Cc1c(N)sc2c(F)ccc(-c3c(C(F)(F)F)cc4c(N5CC6CCC(C5)N6)nc(OCC5(C(F)F)COC5)nc4c3F)c12]

Example 48 was prepared by similar procedure as described in Example 32 by replacing (3-ethyloxetan-3-yl)methanol with (3-(difluoromethyl)oxetan-3-yl)methanol to give the title product (101 mg) which was furtherly separated by chiral-HPLC (Prep-HPLC Equipment: Prep-HPLC-Gilson; Column: IE, 20 mm×250 mm. 5 μm; Mobile Phase A: n-Hexane, Mobile Phase B: EtOH (0.2% 2M NH₃ in MeOH); Flow Rate: 18 mL/min; Temperature: 25° C.; Gradient: 40% B in 14 min; Retention time—isomer 1: 6.5 min; Retention time—isomer 2: 11.5 min) to give a couple of atropisomers. Isomer 1 (42.0 mg, 30.4%): ¹H NMR (500 MHz, CD₃OD) δ 8.13 (s, 1H). 7.23-7.20 (m, 1H), 7.03-6.99 (m, 1H). 6.37-6.15 (m, 1H), 4.81 (s, 2H), 4.78-4.77 (m, 2H), 4.70-4.65 (m, 2H), 4.59-4.57 (m, 1H), 4.50-4.47 (m, 1H), 3.74-3.66 (m, 4H), 1.90-1.81 (m, 4H); MS (EST, m/e) [M+1]⁺ 653.7. Isomer 2 (40.0 mg, 29.0%): ¹H NMR (500 MHz, CD₃OD) δ 8.13 (s, 1H). 7.23-7.20 (m, 1H), 7.03-6.99 (m, 1H), 6.38-6.15 (m, 1H), 4.80 (s, 2H), 4.78-4.77 (m, 2H), 4.70-4.65 (m, 2H), 4.58-4.56 (m, 1H), 4.49-4.47 (m, 1H), 3.73-3.65 (m, 4H), 1.90-1.81 (m, 4H); MS (ESI, m/e) [M+1]⁺ 653.6.

### Example 49: 3-(((7-(2-amino-3-cyano-7-fluorobenzo[b]thiophen-4-yl)-4-(3,8-diazabicyclo[3.2.1]octan-3-yl)-8-fluoro-6-(trifluoromethyl)quinazolin-2-yl)oxy) methyl)oxetane-3-carbonitrile

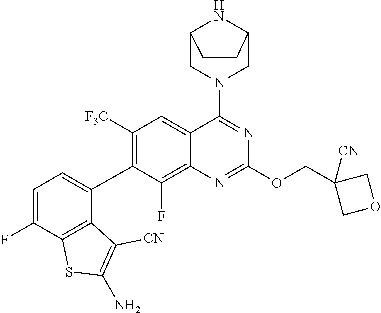 [SMILES: N#Cc1c(N)sc2c(F)ccc(-c3c(C(F)(F)F)cc4c(N5CC6CCC(C5)N6)nc(OCC5(C#N)COC5)nc4c3F)c12]

Example 49 was prepared by similar procedure as described in Example 3 by replacing (3-methoxytetrahydrofuran-3-yl)methanol with 3-(hydroxymethyl) oxetane-3-carbonitrile to give the title product (24.91 mg). ¹H NMR (500 MHz, CD₃OD) δ 8.14 (s, 1H), 7.23-7.20 (m, 1H), 7.03-6.99 (m, 111), 4.97-4.95 (m, 4H), 4.78-4.77 (m, 2H), 4.61-4.58 (m, 1H), 4.51-4.48 (m, 1H), 3.74-3.66 (m, 4H), 1.89-1.78 (m, 4H). MS (ESI, m/e) [M+H]⁺ 628.4.

### Example 50: 4-(4-(3,8-diazabicyclo[3.2.1]octan-3-yl)-8-fluoro-2-((4-(hydroxy methyl)tetrahydro-2H-pyran-4-yl)methoxy)-6-(trifluoromethyl) quinazolin-7-yl)-2-amino-7-fluorobenzo[b]thiophene-3-carbonitrile

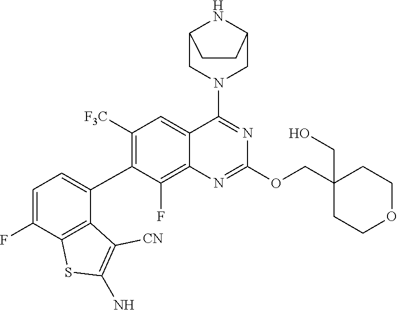 [SMILES: N#Cc1c(N)sc2c(F)ccc(-c3c(C(F)(F)F)cc4c(N5CC6CCC(C5)N6)nc(OCC5(CO)CCOCC5)nc4c3F)c12]

### Step 1: tert-butyl 3-(7-(2-((tert-butoxycarbonyl)amino)-3-cyano-7-fluorobenzo [b]thiophen-4-yl)-8-fluoro-2-((4-(hydroxymethyl)tetrahydro-2H-pyran-4-yl)methoxy)-6-(trifluoromethyl)quinazolin-4-yl)-3,8-diazabicyclo[3.2.1]octane-8-carboxylate

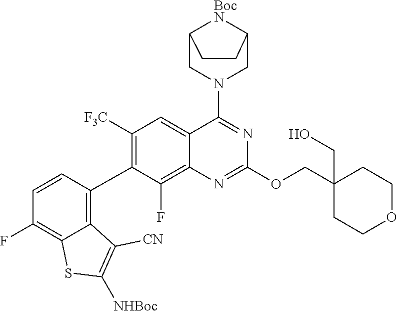 [SMILES: CC(C)(C)OC(=O)Nc1sc2c(F)ccc(-c3c(C(F)(F)F)cc4c(N5CC6CCC(C5)N6C(=O)OC(C)(C)C)nc(OCC5(CO)CCOCC5)nc4c3F)c2c1C#N]

To a solution of (tetrahydro-2H-pyran-4,4-diyl)dimethanol (44 mg, 0.3 mmol) in THE (10 mL) was added sodium hydride (15 mg, 0.60 mmol) at room temperature. The resulting mixture was stirred at room temperature for 1 h. Then, tert-butyl 3-(7-(2-((tert-butoxycarbonyl)amino)-3-cyano-7-fluorobenzo[b]thiophen-4-yl)-2,8-difluoro-6-(trifluoromethyl)quinazolin-4-yl)-3,8-diazabicyclo[3.2.1]octane-8-carboxylate (73.4 mg, 0.10 mmol) was added to the reaction mixture and stirred at room temperature for 1 h. After completion, the reaction mixture was diluted with EtOAc (50 mL) and washed with saturated NaCl (15 mL×3) aqueous solution. The organic layer was dried over anhydrous Na₂SO₄, filtered and the filtrate was concentrated to give the residue. The residue was purified by Prep-TLC (Petroleum ether:EtOAc=1:1) to give the title product (40 mg, 46.5%). MS (ESI, m/e) [M+1]+860.5.

### Step 2: 4-(4-(3,8-diazabicyclo[3.2.1]octan-3-yl)-8-fluoro-2-((4-(hydroxymethyl) tetrahydro-2H-pyran-4-yl)methoxy)-6-(trifluoromethyl) quinazolin-7-yl)-2-amino-7-fluorobenzo[b]thiophene-3-carbonitrile

To a solution of tert-butyl 3-(7-(2-((tert-butoxycarbonyl)amino)-3-cyano-7-fluorobenzo[b]thiophen-4-yl)-8-fluoro-2-((4-(hydroxymethyl)tetrahydro-2H-pyran-4-yl)methoxy)-6-(trifluoromethyl)quinazolin-4-yl)-3,8-diazabicyclo[3.2.1]octane-8-carboxylate (40 mg, 0.0465 mmol) in DCM (2 mL) was added TFA (2 mL) at room temperature. The resulting mixture was stirred at room temperature for 3 h. After completion, the reaction mixture was concentrated to give the residue. The residue was purified by prep-HPLC to give the title product (15.0 mg, 48.9%). ¹H NMR (500 MHz, CD₃OD) δ 8.11 (s, 1H), 7.24-7.19 (m, 1H), 7.04-7.00 (m, 1H), 4.71-4.68 (m, 1H), 4.60-4.58 (m, 1H). 4.51-4.45 (m, 2H), 4.06-4.02 (m, 2H), 3.90-3.70 (m, 6H), 3.64 (s, 2H), 2.09-2.01 (m, 4H), 1.69-1.58 (m, 4H). MS (ESI, m/e) [M+1]+661.5.

### Example 51: 4-(4-(3,8-diazabicyclo[3.2.1]octan-3-yl)-2-((3-(cyanomethyl) oxetan-3-yl)methoxy)-8-fluoro-6-(trifluoromethyl)quinazolin-7-yl)-2-amino-7-fluoro benzo[b]thiophene-3-carbonitrile

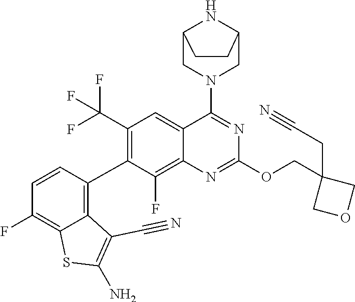 [SMILES: N#CCC1(COc2nc(N3CC4CCC(C3)N4)c3cc(C(F)(F)F)c(-c4ccc(F)c5sc(N)c(C#N)c45)c(F)c3n2)COC1]

Example 51 was prepared by similar procedure as described in Example 6 by replacing 3-(hydroxymethyl)tetra hydrofuran-3-ol with 2-(3-(hydroxymethyl)oxetan-3-yl)acetonitrile to give the title product (25 mg, 40.2% yield, Formate). ¹H NMR (500 MHz, CD₃OD) δ 8.47 (s, 1H), 8.13 (s, 1H), 7.23-7.20 (m, 1H), 7.04-7.00 (m, 1H), 4.79-4.75 (m, 2H), 4.70-4.68 (m, 3H), 4.63-4.61 (m, 3H), 4.01-3.95 (m, 2H), 3.85-3.77 (m, 2H), 3.06 (s, 2H), 2.04-1.95 (m, 4H). MS (ESI, m/e) [M+1]⁺ 642.5.

### Example 52: 4-(4-(3,8-diazabicyclo[3.2.1]octan-3-yl)-6-ethyl-8-fluoro-2-((1-methyl-2-oxabicyclo[2.1.1]hexan-4-yl)methoxy)quinazolin-7-yl)-2-amino-7-fluorobenzo[b]thiophene-3-carbonitrile

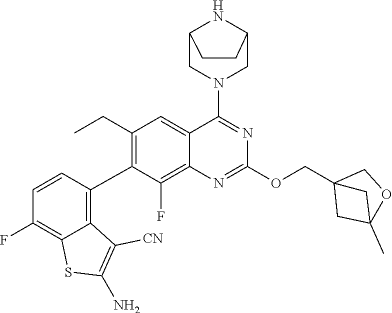 [SMILES: CCc1cc2c(N3CC4CCC(C3)N4)nc(OCC34COC(C)(C3)C4)nc2c(F)c1-c1ccc(F)c2sc(N)c(C#N)c12]

### Step 1: tert-butyl 3-(7-bromo-2-chloro-8-fluoro-6-iodoquinazolin-4-yl)-3,8-diazabicyclo[3.2.1]octane-8-carboxylate

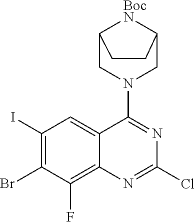 [SMILES: CC(C)(C)OC(=O)N1C2CCC1CN(c1nc(Cl)nc3c(F)c(Br)c(I)cc13)C2]

To a mixture of 7-bromo-2,4-dichloro-8-fluoro-6-iodoquinazoline (0.2 g, 0.47 mmol) and DIPEA (0.12 g. 0.94 mmol) in DCM (10 mL) was cooled to 0° C., tert-butyl 3,8-diazabicyclo[3.2.1]octane-8-carboxylate was added, the mixture was stirred for 2 h at 0° C., diluted with DCM (10 mL), repeated operation three times, washed with brine (10 mL), filtered, the filtrate was concentrated and purified by chromatography column on silica to give the title product (0.23 g, 82.1%). MS (ESI, m/e) [M+1]+598.4.

### Step 2: tert-butyl 3-(7-bromo-2,8-difluoro-6-iodoquinazolin-4-yl)-3,8-diazabicyclo[3.2.1]octane-8-carboxylate

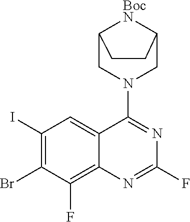 [SMILES: CC(C)(C)OC(=O)N1C2CCC1CN(c1nc(F)nc3c(F)c(Br)c(I)cc13)C2]

To a solution of tert-butyl 3-(7-bromo-2-chloro-8-fluoro-6-iodoquinazolin-4-yl)-3,8-diazabicyclo[3.2.1]octane-8-carboxylate (0.1 g, 0.16 mmol) in DMSO (5 mL) was added KF (0.039 g, 0.64 mmol), stirred at 90° C. for 5-6 h. The resulting mixture was cooled to rt, diluted with EtOAc (10 mL), repeated operation three times, washed with brine (10 mL), and filtered. The filtrate was concentrated and purified by chromatography column on silica to give the title product (0.08 g, 82.4%). MS (ESI, m/e) [M+1]+581.3.

### Step 3: tert-butyl 3-(7-bromo-2,8-difluoro-6-vinylquinazolin-4-yl)-3,8-diazabicyclo[3.2.1]octane-8-carboxylate

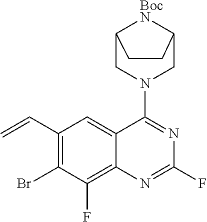 [SMILES: C=Cc1cc2c(N3CC4CCC(C3)N4C(=O)OC(C)(C)C)nc(F)nc2c(F)c1Br]

A mixture of tert-butyl 3-(7-bromo-2,8-difluoro-6-iodoquinazolin-4-yl)-3,8-diazabicyclo[3.2.1]octane-8-carboxylate (620 mg, 1.06 mmol), trifluoro(vinyl)-14-borane, potassium salt (428 mg, 3.2 mmol), NaHCO₃ (268 mg, 3.2 mmol), dioxane (20 mL) and H₂O (2 mL) was added Pd(dppf)Cl₂DCM (173 mg, 0.21 mmol), degassed with N₂ for 3 times and stirred at 70° C. for 2 hours, the resulting mixture was filtered, the filtrate was concentrated and purified by column chromatography to give the title product (450 mg, 87.8%). MS (ESI, m/e) [M+H]⁺ 481.1.

### Step 4: tert-butyl 3-(7-bromo-6-ethyl-2,8-difluoroquinazolin-4-yl)-3,8-diazabicyclo[3.2.1]octane-8-carboxylate

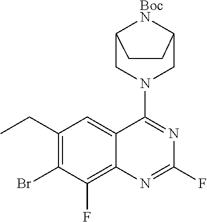 [SMILES: CCc1cc2c(N3CC4CCC(C3)N4C(=O)OC(C)(C)C)nc(F)nc2c(F)c1Br]

To a solution of tert-butyl 3-(7-bromo-2,8-difluoro-6-vinylquinazolin-4-yl)-3,8-diazabicyclo[3.2.1]octane-8-carboxylate (250 mg, 0.51 mmol) in EtOAc (15 mL) was added Pt/C (10%) (25 mg), the mixture was stirred at rt for 48 hours, the resulting mixture was filtered, the filtrate was concentrated and purified by column chromatography to give the title product (160 mg, 64.2%). MS (ESI, m/e) [M+H]⁺ 483.1.

### Step 5: tert-butyl 3-(7-bromo-6-ethyl-8-fluoro-2-((1-methyl-2-oxabicyclo[2.1.1]hexan-4-yl)methoxy)quinazolin-4-yl)-3,8-diazabicyclo[3.2.1]octane-8-carboxylate

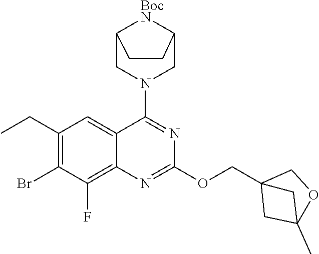 [SMILES: CCc1cc2c(N3CC4CCC(C3)N4C(=O)OC(C)(C)C)nc(OCC34COC(C)(C3)C4)nc2c(F)c1Br]

A solution of (1-methyl-2-oxabicyclo[2.1.1]hexan-4-yl)methanol (100 mg, 0.78 mmol) in THE (30 mL) was cooled to 0° C., NaH (29 mg, 0.74 mmol) was added one portion, stirred at 0° C. for 30 min, then added into tert-butyl 3-(7-bromo-6-ethyl-2,8-difluoroquinazolin-4-yl)-3,8-diazabicyclo[3.2.1]octane-8-carboxylate (160 mg, 0.33 mmol). The resulting mixture was stirred at rt for 4-5 hours, then quenched with H₂O (20 mL), extracted with EtOAc (30 mL) for three times, washed with brine (20 mL), dried over Na₂SO₄, filtered, the filtrate was concentrated and purified by column chromatography to give the title product (150 mg, 76.9%). MS (EST, m/e) [M+H]⁺ 591.3.

### Step 6: tert-butyl 3-(7-(2-((tert-butoxycarbonyl)amino)-3-cyano-7-fluorobenzo[b]thiophen-4-yl)-6-ethyl-8-fluoro-2-((1-methyl-2-oxabicyclo[2.1.1]hexan-4-yl)methoxy)quinazolin-4-yl)-3,8-diazabicyclo[3.2.1]octane-8-carboxylate

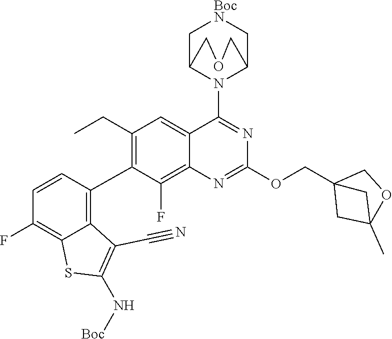 [SMILES: CCc1cc2c(N3C4COCC3CN(C(=O)OC(C)(C)C)C4)nc(OCC34COC(C)(C3)C4)nc2c(F)c1-c1ccc(F)c2sc(NC(=O)OC(C)(C)C)c(C#N)c12]

A mixture of tert-butyl 3-(7-bromo-6-ethyl-8-fluoro-2-((1-methyl-2-oxabicyclo[2.1.1]hexan-4-yl)methoxy)quinazolin-4-yl)-3,8-diazabicyclo[3.2.1]octane-8-carboxylate (50 mg, 0.084 mmol), tell-butyl (3-cyano-7-fluoro-4-(4,4,5,5-tetramethyl-1,3,2-dioxaborolan-2-yl)benzo[b]thiophen-2-yl)carbamate (140 mg, 0.034 mmol), Cs₂CO₃ (82 mg, 0.25 mmol), DPEphosPdCl₂ (12 mg, 0.017) was added toluene (10 mL), degassed with N₂ for 3 times and stirred at 100° C. for 5 hours, the resulting mixture was filtered, the filtrate was concentrated and purified by column chromatography to give the title product (25 mg, 36.7%), MS (EST, m/e) [M+H]⁺ 803.6.

### Step 7: 4-(4-(3,8-diazabicyclo[3.2.1]octan-3-yl)-6-ethyl-8-fluoro-2-((1-methyl-2-oxabicyclo[2.1.1]hexan-4-yl)methoxy)quinazolin-7-yl)-2-amino-7-fluorobenzo[b]thiophene-3-carbonitrile

To a solution of tert-butyl 3-(7-(2-((tart-butoxycarbonyl)amino)-3-cyano-7-fluorobenzo[b]thiophen-4-yl)-6-ethyl-8-fluoro-2-((1-methyl-2-oxabicyclo[2.1.1]hexan-4-yl)methoxy)quinazolin-4-yl)-3,8-diazabicyclo[3.2.1]octane-8-carboxylate (80 mg, 0.1 mmol) in DCM (5 mL) was added TFA (1 mL) at room temperature. The resulting mixture was stirred at room temperature for 2 hours. After completion. The reaction mixture was concentrated to give the residue. The residue was purified by prep-HPLC to give the title product (3.0 mg, 5.0%, Formate), ¹H NMR (500 MHz, CD₃OD) S 8.49 (s, 0.5H), 7.61 (s, 1H), 7.18-7.15 (m, 1H), 7.05-7.02 (m, 1H), 4.74-4.68 (m, 2H), 4.58-4.56 (m, 1H), 4.52-4.49 (m, 1H), 4.00 (s, 2H), 3.79 (s, 2H), 3.73-3.66 (m, 2H), 2.62-2.42 (m, 2H), 2.08-2.04 (m, 4H), 1.81-1.80 (m, 2H), 1.62-1.61 (m, 2H), 1.42 (s, 3H), 1.10-1.07 (m, 3H). MS (ESI, m/e) [M+1]+603.4.

### Example 53: 4-(4-(3,8-diazabicyclo[3.2.1]octan-3-yl)-8-fluoro-2-((3-(fluoro methyl)oxetan-3-yl)methoxy)-6-(trifluoromethyl)quinazolin-7-yl)-2-amino-7-fluoro benzo[b]thiophene-3-carbonitrile

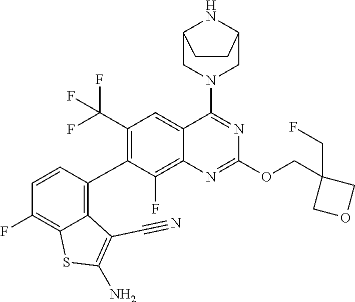 [SMILES: N#Cc1c(N)sc2c(F)ccc(-c3c(C(F)(F)F)cc4c(N5CC6CCC(C5)N6)nc(OCC5(CF)COC5)nc4c3F)c12]

Example 53 was prepared by similar procedure as described in Example 6 by replacing 3-(hydroxymethyl)tetra hydrofuran-3-ol with (3-(fluoromethyl)oxetan-3-yl)methanol to give the title product (17 mg, 36.2% yield). ¹H NMR (500 MHz, CD₃OD) δ 8.13 (s, 1H), 7.23-7.20 (m, 1H), 7.04-6.99 (m, IF), 4.83-4.81 (m, IF), 4.75-4.71 (m, 3H), 4.69-4.66 (m, 2H), 4.63-4.60 (m, 3H), 4.54-4.51 (m, 1H), 3.81-3.70 (m, 411), 1.97-1.83 (m, 4H). MS (ESI, m/e) [M+1]⁺ 635.5.

### Example 54: 4-(4-(3,8-diazabicyclo[3.2.1]octan-3-yl)-8-fluoro-2-((3-methyl-1-(1-methyl-1H-imidazol-2-yl)azetidin-3-yl)methoxy)-6-(trifluoromethyl)quinazolin-7-yl)-2-amino-7-fluorobenzo[b]thiophene-3-carbonitrile

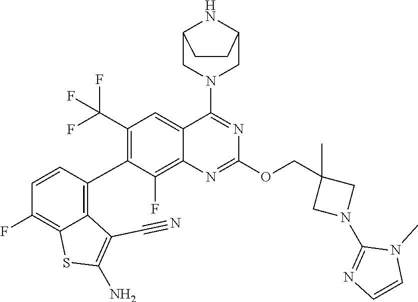 [SMILES: Cn1ccnc1N1CC(C)(COc2nc(N3CC4CCC(C3)N4)c3cc(C(F)(F)F)c(-c4ccc(F)c5sc(N)c(C#N)c45)c(F)c3n2)C1]

Example 54 was prepared by similar procedure as described in Example 46 by replacing azetidin-3-ylmethanol hydrochloride with (3-methylazetidin-3-yl)methanol hydrochloride to give the title product (4 mg, 16.7% yield). ¹H NMR (500 MHz, CD₃OD) δ 8.12 (s, 1H), 7.22-7.20 (m, 1H), 7.03-7.00 (m, 1H), 6.77-6.73 (m, 2H), 4.67-4.55 (m, 4H), 4.22-4.19 (m, 2H), 3.97-3.89 (m, 4H), 3.83-3.75 (m, 2H), 3.50 (s, 3H), 2.00-1.92 (m, 4H), 1.51 (s, 3H). MS (ESI, m/e) [M+1]⁺ 696.5.

### Example 55: 4-(4-(3,8-diazabicyclo[3.2.1]octan-3-yl)-8-fluoro-2-((6-methyl-2-oxaspiro[3.3]heptan-6-yl)methoxy)-6-(trifluoromethyl)quinazolin-7-yl)-2-amino-7-fluorobenzo[b]thiophene-3-carbonitrile

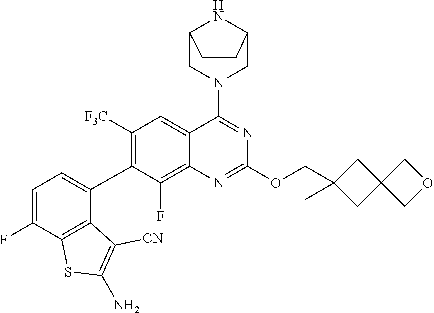 [SMILES: CC1(COc2nc(N3CC4CCC(C3)N4)c3cc(C(F)(F)F)c(-c4ccc(F)c5sc(N)c(C#N)c45)c(F)c3n2)CC2(COC2)C1]

Example 55 was prepared by similar procedure as described in Example 1 (step 6 and step 7) by replacing (1-(difluoromethyl)-2-oxabicyclo[2.1.1]hexan-4-yl) methanol with (6-methyl-2-oxaspiro[3.3]heptan-6-yl)methanol to give the title product (15 mg, 48.3%). ¹H NMR (500 MHz, CD₃OD) δ 8.10 (s, 1H), 7.21-7.20 (m, 1H), 7.03-6.99 (m, 1H), 4.74-4.71 (m, 4H), 4.59-4.57 (m, 1H). 4.49-4.46 (m, 1H), 4.22 (s, 2H). 3.79-3.67 (m, 4H), 2.40-2.37 (m, 2H), 2.07-2.05 (m, 2H), 1.89 (s. 4H), 1.19 (s, 3H). MS (ESI, m/e) [M+H]⁺ 657.5

### Example 56: 4-(4-(3,8-diazabicyclo[3.2.1]octan-3-yl)-8-fluoro-2-((1-(methoxymethyl)-3,3-dimethyl-2-oxabicyclo[2.1.1]hexan-4-yl)methoxy)-6-(trifluoromethyl)quinazolin-7-yl)-2-amino-7-fluorobenzo[b]thiophene-3-carbonitrile

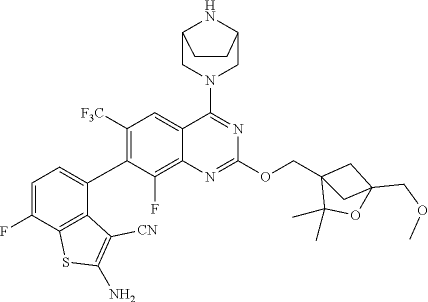 [SMILES: COCC12CC(COc3nc(N4CC5CCC(C4)N5)c4cc(C(F)(F)F)c(-c5ccc(F)c6sc(N)c(C#N)c56)c(F)c4n3)(C1)C(C)(C)O2]

### Step 1: methyl 1-(2-hydroxypropan-2-yl)-3-methylenecyclobutane-1-carboxylate

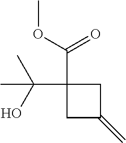

To a mixture of diisopropylamine (1.11 g, 0.011 mol) in THF (50 mL) was added dropwise n-BuLi (4.4 mL, 2.5 N, 0.011 mol) at -78° C. Then, it was warmed to 0 Ct for 30 mins. After being re-cooled to −78 t, methyl 3-methylenecyclobutane-1-carboxylate (1.27 g, 0.01 mol) was added into the reaction mixture and stirred at -78<C for 1 h. Then, acetone (1.17 g, 0.02 mol) was added dropwise into the reaction mixture and warmed to rt for 2 h. After completion, the reaction mixture was quenched by 50 mL H₂O, extracted with EtOAc (50 mL×3). The combined organic layers were washed with saturated NaCl aqueous solution (100 mL), dried over anhydrous Na₂SO₄, filtered and the filtrate was concentrated to give the crude title product (1.15 g, 6.25 mmol). ¹H NMR (500 MHz, CDCl₃) δ 4.84 (s, 2H), 3.77 (s, 3H), 3.11 (s, 1H), 3.07-3.04 (m, 2H), 2.93-2.90 (m, 2H), 1.24 (s, 6H).

### Step 2: methyl 1-(iodomethyl)-3,3-dimethyl-2-oxabicyclo[2.1.1]hexane-4-carboxylate

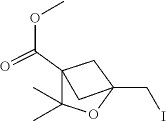

To a mixture of methyl 1-(2-hydroxypropan-2-yl)-3-methylenecyclobutane-1-carboxylate (650 mg, 3.53 mmol) in tert-butyl methyl ether (20 mL) and H₂O (10 mL) was added I₂ (1.79 g, 7.06 mmol) and NaHCO₃ (594 mg, 7.06 mmol) at rt. Then, it was stirred at rt for 4 h. After completion, the reaction mixture was diluted with tert-butyl methyl ether (100 mL) and quenched by saturated Na₂S₂O₃ aqueous solution (100 mL). The combined organic layers were washed with saturated NaCl aqueous solution (100 mL), dried over anhydrous Na₂SO₄, filtered and the filtrate was concentrated to give the crude title product (720 mg, 2.32 mmol). ¹H NMR (500 MHz, CDCl₃) δ 3.72 (s, 3H), 3.38 (s, 2H), 2.23-2.22 (m, 2H), 2.05-2.04 (m, 2H), 1.43 (s, 6H).

### Step 3: 1-(methoxymethyl)-3,3-dimethyl-2-oxabicyclo[2.1.1]hexane-4-carboxylic acid

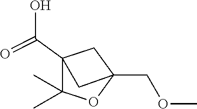

To a mixture of methyl 1-(iodomethyl)-3,3-dimethyl-2-oxabicyclo[2.1.1]hexane-4-carboxylate (600 mg, 1.94 mmol) in HPLC grade MeOH (30 mL) was added sodium methoxide (3.14 g, 58.2 mmol) at 0° C. Then, it was heated to reflux for 24 h. After completion, the reaction mixture was concentrated. The residue was diluted with H₂O (100 mL). The aqueous layer was extracted with tert-butyl methyl ether (50 mL×2). Then, the aqueous layer was adjusted to PH=1-2 using 1 N HCl aqueous solution, and extracted with DCM (70 mL×3). The combined organic layers were washed with saturated NaCl aqueous solution (100 mL), dried over anhydrous Na₂SO₄, filtered and the filtrate was concentrated to give the crude title product as a brown solid (243 mg, 1.22 mmol, purity %=75%). ¹H NMR (500 MHz, CDCl₃) δ 3.58 (s, 2H), 3.40 (s, 3H), 2.25-2.24 (m, 2H), 2.11-2.05 (m, 2H), 1.46 (s, 6H).

### Step 4: (1-(methoxymethyl)-3,3-dimethyl-2-oxabicyclo[2.1.1]hexane-4-yl)methanol

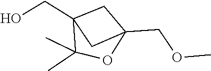

To a mixture of 1-(methoxymethyl)-3,3-dimethyl-2-oxabicyclo[2.1.1]hexane-4-carboxylic acid (190 mg, 0.95 mmol) in THF (5 mL) was added dropwise BH₃•THF (1.9 mL, 1 N, 1.90 mmol) at 0° C. Then, it was stirred at rt for 3 h. After completion, the reaction mixture was quenched by MeOH (10 mL) at 0° C. and concentrated to give the crude product. It was purified by silica gel column chromatography and eluted with 0-1.2% MeOH in DCM to give the title product as the colorless oil (60 mg, 0.32 mmol). ¹H NMR (500 MHz, CDCl₃) δ 3.79 (s, 2H), 3.58 (s, 2H), 3.40 (s, 3H), 1.89-1.88 (m, 2H), 1.71-1.70 (m, 2H), 1.32 (s, 6H).

### Step 5: tert-butyl 3-(7-(2-((tert-butoxycarbonyl)amino)-3-cyano-7-fluorobenzo[b]thiophen-4-yl)-8-fluoro-2-((1-(methoxymethyl)-3,3-dimethyl-2-oxabicyclo[2.1.1]hexan-4-yl)methoxy)-6-(trifluoromethyl)quinazolin-4-yl)-3,8-diazabicyclo[3.2.1]octane-8-carboxylate

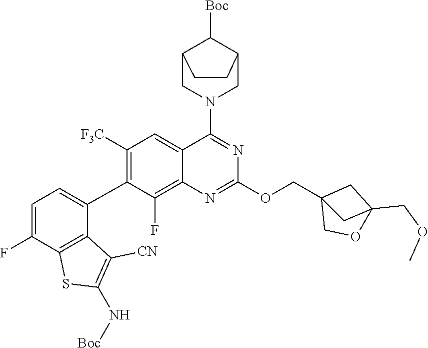 [SMILES: COCC12CC(COc3nc(N4CC5CCC(C4)C5C(=O)OC(C)(C)C)c4cc(C(F)(F)F)c(-c5ccc(F)c6sc(NC(=O)OC(C)(C)C)c(C#N)c56)c(F)c4n3)(CO1)C2]

To a mixture of tert-butyl 3-(7-(2-((tert-butoxycarbonyl)amino)-3-cyano-7-fluorobenzo[b]thiophen-4-yl)-2,8-difluoro-6-(trifluoromethyl)quinazolin-4-yl)-3,8-diazabicyclo[3.2.1]octane-8-carboxylate (70.0 mg, 0.095 mmol) in THE (5 mL) was added (1-(methoxymethyl)-3,3-dimethyl-2-oxabicyclo[2.1.1]hexan-4-yl)methanol (35 mg, 0.19 mmol), and sodium hydride (11.4 mg, 0.29 mmol) at 0° C. The reaction mixture was stirred at room temperature for 2 h. After completion, the reaction mixture was quenched by H₂O (30 mL) and extracted with EtOAc (30 mL×3). The combined organic layers were washed with saturated NaCl aqueous solution (100 mL), dried over anhydrous Na₂SO₄, and filtered. The filtrate was concentrated to give the crude title product. It was purified by silica gel column chromatography and eluted with 0-20% EtOAc in petroleum ether to give the title product as the yellow solid (45 mg, 0.05 mmol). MS (ESI, mile) [M+1]⁺ 901.7.

### Step 6: 4-(4-(3,8-diazabicyclo[3.2.1]octan-3-yl)-8-fluoro-2-((1-(methoxymethyl)-3,3-dimethyl-2-oxabicyclo[2.1.1]hexan-4-yl)methoxy)-6-(trifluoromethyl)quinazolin-7-yl)-2-amino-7-fluorobenzo[b]thiophene-3-carbonitrile

To a solution of tert-butyl 3-(7-(2-((tert-butoxycarbonyl)amino)-3-cyano-7-fluorobenzo[b]thiophen-4-yl)-8-fluoro-2-((1-(methoxymethyl)-3,3-dimethyl-2-oxabicyclo[2.1.1]hexan-4-yl)methoxy)-6-(trifluoromethyl)quinazolin-4-yl)-3,8-diazabicyclo[3.2.1]octane-8-carboxylate (45 mg, 0.05 mmol) in DCM (3 mL) was added TFA (3 mL) at room temperature. The resulting mixture was stirred at room temperature for 2 hours. After completion. The reaction mixture was concentrated to give the residue. The residue was purified by prep-HPLC to give the title product as the yellow solid (25 mg, 71% yield). ¹H NMR (500 MHz, CD₃OD) δ 8.11 (s, 1H), 7.23-7.20 (m, 1H), 7.03-7.00 (m, 1H). 4.72-4.54 (m, 4H), 4.02 (s, 2H), 3.84-3.81 (m, 1H), 3.78-3.76 (m, 1H), 3.55 (s, 2H), 3.36 (s, 3H), 2.02 (s, 4H), 1.97-1.96 (m, 2H), 1.84 (s, 2H), 1.36 (s, 6H). MS (ESI, mile) [M+1]⁺ 701.4.

### Example 57: 4-(4-(3,8-diazabicyclo[3.2.1]octan-3-yl)-8-fluoro-2-((3-(hydroxymethyl)oxetan-3-yl)methoxy)-6-(trifluoromethyl)quinazolin-7-yl)-2-amino-7-fluorobenzo[b]thiophene-3-carbonitrile

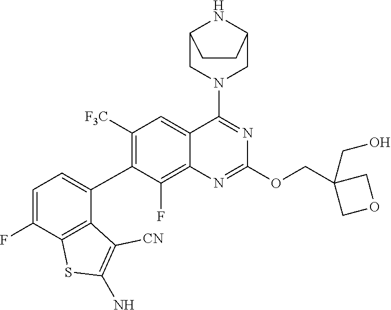 [SMILES: N#Cc1c(N)sc2c(F)ccc(-c3c(C(F)(F)F)cc4c(N5CC6CCC(C5)N6)nc(OCC5(CO)COC5)nc4c3F)c12]

### Step 1: tert-butyl 3-(7-(2-((tert-butoxycarbonyl)amino)-3-cyano-7-fluorobenzo[b]thiophen-4-yl)-8-fluoro-2-((3-(hydroxymethyl)oxetan-3-yl)methoxy)-6-(trifluoromethyl)quinazol in-4-yl)-3,8-diazabicyclo[3.2.1]octane-8-carboxylate

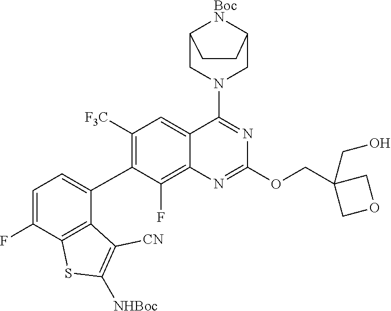 [SMILES: CC(C)(C)OC(=O)Nc1sc2c(F)ccc(-c3c(C(F)(F)F)cc4c(N5CC6CCC(C5)N6C(=O)OC(C)(C)C)nc(OCC5(CO)COC5)nc4c3F)c2c1C#N]

To a solution of tert-butyl 3-(7-(2-((tert-butoxycarbonyl)amino)-3-cyano-7-fluoro benzo[b]thiophen-4-yl)-2,8-difluoro-6-(trifluoromethyl)quinazolin-4-yl)-3,8-diaza bicyclo[3.2.1]octane-8-carboxylate (150 mg, 0.20 mmol) and oxetane-3,3-diyldimethanol (110 mg, 0.93 mmol) in THE (3 mL) was added NaH (10 mg, 0.25 mmol) at rt. The resulting mixture was stirred at rt for 4 hours. After completion, the reaction mixture was diluted with EtOAc (50 mL) and washed with saturated NaCl (15 mL) aqueous solution. The organic layer was dried over anhydrous Na₂SO₄, filtered and the filtrate was concentrated to give the residue. It was purified by silica gel column chromatography and eluted with 0-100% EtOAc in petroleum ether to give the title product (100 mg, 0.12 mmol). MS (ESI, m/e) [M+1]⁺ 833.5.

### Step 2: 4-(4-(3,8-diazabicyclo[3.2.1]octan-3-yl)-8-fluoro-2-((3-(hydroxymethyl)oxetan-3-yl)methoxy)-6-(trifluoromethyl)quinazolin-7-yl)-2-amino-7-fluorobenzo[b]thiophene-3-carbonitrile

To a solution of tert-butyl 3-(7-(2-((tert-butoxycarbonyl)amino)-3-cyano-7-fluorobenzo[b]thiophen-4-yl)-8-fluoro-2-((3-(hydroxymethyl)oxetan-3-yl)methoxy)-6-(trifluoromethyl)quinazolin-4-yl)-3,8-diazabicyclo[3.2.1]octane-8-carboxylate (50 mg, 0.06 mmol) in DCM (4 mL) was added TFA (2 mL) at room temperature. The reaction mixture was stirred at room temperature for 4 h. The solvent was concentrated. The residue was dissolved in EtOAc (20 mL) and saturated Na₂CO₃ aqueous solution (20 mL) was added. The solution was stirred at rt for 10 min and extracted by EtOAc (40 mL×2). The combined organic phase was washed with brine (20 mL), dried by Na₂SO₄, and then concentrated under vacuum. It was purified by prep-HPLC to give the title product (20 mg, 53% yield,). ¹H NMR (500 MHz, CD₃OD) δ 8.49 (s, 0.5H), 8.12 (s, 1H), 7.23-7.20 (m, 1H), 7.03-7.00 (m, 1H), 4.73-4.68 (m, 3H), 4.64-4.63 (m, 2H), 4.56-4.55 (m, 3H), 3.91 (s, 4H), 3.83-3.74 (m, 2H), 1.97 (s, 4H). MS (ESI, m/e) [M+1]⁺ 633.4.

### Assays

### KRAS Probe Displacement Assay

This assay was used to identify compounds which bind to GDP-loaded KRAS protein and are able to displace a biotinylated probe occupying the KRAS binding site. GST-tagged GDP-loaded WT KRAS (amino acids 1-188) and GST-tagged GDP-loaded KRAS G12D (amino acids 1-188) were expressed in E. coli and purified in house. All protein and reaction solutions were prepared in assay buffer containing 50 mM HEPES pH7.5, 50 mM NaCl, 1 mM MgCl₂, 1 mM TCEP, 0.01% BSA, and 0.008% Brij-35. Purified WT KRAS (3 nM final concentration) or KRAS G12D protein (0.5 nM final concentration) was incubated with a 3-fold serially diluted compound in the assay plate (384 well microplate, black, Corning). Plates are incubated at 24° C. for 1 hr. Following the incubation, biotinylated probe 1 (60 nM final assay concentration) for WT KRAS and biotinylated probe 2 (4 nM final assay concentration) for KRAS G12D was added to the assay plate, respectively. After 1 hr incubation at 24C, Mab Anti-GST-Th cryptate (Cisbio) and Streptavidin-XL665 (Cisbio) were added and further incubated at 24° C. for another 1 hr. The TR-FRET signals (ex337 nm, em665 nm/620 nm) were read on BMG PHERAstar FSX instrument. The inhibition percentage of KRAS protein binding with biotinylated probe in presence of increasing concentrations of compounds was calculated based on the ratio of fluorescence at 665 nm to that at 620 nm. The IC₅₀ value of each compound was calculated from fitting the data to the four-parameter logistic model by Dotmatics.

pERK assay

AsPC-1 cell line was used in this study. Cells were maintained in RPMI-1640 supplemented with 10% fetal bovine serum (Thermo Fisher), 50 units/mL penicillin and streptomycin (Thermo Fisher) and kept at 37° C. in a humidified atmosphere of 5% CO₂ in air. Cells were reinstated from frozen stocks that were laid down within 30 passages from the original cells purchased. 30000 cells per well were seeded into a 96-well plate and incubated overnight. Cells were treated with a 10-point dilution series. The final compound concentration is from 0 to 10 μM. After 2 h compound treatment, cells were lysed, and the pERK1/2(THR202/TYR204) level in the cell lysates was detected by HTRF kit (Cisbio). In brief, a total of 16 μL of cell lysate from each well of a 96-well plate was transferred to a 384-well white assay plate. Lysate from each well was incubated with 2 μL of Eu3+-cryptate (donor) labeled anti-phospho-ERK1/2 and 2 μL of D₂ (acceptor) labeled anti-phospho-ERK1/2 antibodies (Cisbio) overnight in dark at room temperature. When donor and acceptor are in close proximity, excitation of the donor with laser triggers a Fluorescence Resonance Energy Transfer (FRET) towards the acceptor, which in turn fluoresces at 655 nm wavelength. FRET signals were measured using a PHERAstar FSX reader (BMG Labtech). IC₅₀ determination was performed by fitting the curve of percent inhibition versus the log of the inhibitor concentration using Dotmatics.

### Activity Table

Each of the compounds in Table 2 was tested in one or more of the biochemical assays provided herein and was found to have activity therein.

TABLE 2

| | KRAS^{WT} | KRAS^{G12D} | |
|---|---|---|---|
| | Probe | Probe | |
| | Displacement | Displacement | AsPC-1 |
| | Assay | Assay | pERK IC₅₀ |
| Example No. | IC50 (nM) | IC50 (nM) | (nM) |
| Example 1-Isomer 1 | >1000 | 124 | 2570 |
| Example 1-Isomer 2 | 24 | 0.57 | 84 |
| Example 2-Isomer 1 | 44 | 0.62 | 160 |
| Example 2-Isomer 2 | >1000 | 160 | >10000 |
| Example 3-Mixture 1 | 923 | 46 | 1729 |
| Example 3-Isomer 3 | 15 | 0.49 | 96 |
| Example 3-Isomer 4 | 39 | 1.6 | 273 |
| Example 4-Isomer 1 | >1000 | 114 | |
| Example 4-Isomer 2 | 15 | 0.38 | 100 |
| Example 5-Isomer 1 | >1000 | 185 | |
| Example 5-Isomer 2 | 29 | 0.92 | 44 |
| Example 6 | 10 | 0.42 | 187 |
| Example 7 | 19 | 0.74 | 112 |
| Example 8 | 25 | 0.67 | 159 |
| Example 9-Isomer 1 | >1000 | 316 | >10000 |
| Example 9-Isomer 2 | 11 | 0.41 | 103 |
| Example 10-Isomer 1 | 582 | 309 | >10000 |
| Example 10-Isomer 2 | >1000 | 198 | >10000 |
| Example 10-Isomer 3 | 7.9 | 0.27 | 42 |
| Example 10-Isomer 4 | 10 | 0.33 | 56 |
| Example 11-Isomer 1 | >1000 | 40 | |
| Example 11-Isomer 2 | 5.6 | 0.16 | 37 |
| Example 12 | 80 | 1.7 | 1733 |
| Example 13 | 7.0 | 0.27 | 146 |
| Example 14-Isomer 1 | >1000 | 147 | |
| Example 14-Isomer 2 | >1000 | 36 | |
| Example 14-Isomer 3 | 14 | 0.44 | 172 |
| Example 14-Isomer 4 | 12 | 0.25 | 138 |
| Example 15 | >1000 | 62 | 7138 |
| Example 16 | 7.8 | 0.38 | 143 |
| Example 17 | 111 | 1.9 | 561 |
| Example 18 | 16 | 0.42 | 146 |
| Example 19 | 24 | 0.61 | 326 |
| Example 20 | 20 | 0.55 | 188 |
| Example 21 | 26 | 0.66 | 760 |
| Example 22-Isomer 1 | >1000 | 822 | >10000 |
| Example 22-Isomer 2 | 9.4 | 0.22 | 72 |
| Example 23 | >1000 | 115 | 334 |
| Example 24 | 1.5 | 0.23 | 200 |
| Example 25 | 30 | 0.85 | 201 |
| Example 26 | 124 | 3.0 | 1458 |
| Example 27 | 54 | 1.4 | 975 |
| Example 28 | 16 | 0.65 | 78 |
| Example 29 | 388 | 76 | >10000 |
| Example 30 | 37 | 0.95 | 71 |
| Example 31 | 15 | 0.61 | 299 |
| Example 32 | 16 | 0.45 | 110 |
| Example 33 | 31 | 0.58 | 163 |
| Example 34 | 17 | 0.43 | 81 |
| Example 35 | 55 | 1.2 | 275 |
| Example 36 | 6.9 | 0.39 | 250 |
| Example 37 | 16 | 0.45 | 119 |
| Example 38 | 17 | 0.37 | 180 |
| Example 39 | 9.6 | 0.27 | 495 |
| Example 40 | 13 | 0.39 | 236 |
| Example 41 | 360 | 8.7 | 860 |
| Example 42 | >1000 | 43 | 4340 |
| Example 43 | 154 | 4.8 | 479 |
| Example 44 | 38 | 1.0 | 211 |
| Example 45 | 16 | 0.45 | 115 |
| Example 46 | 14 | 0.64 | 222 |
| Example 47 | 17 | 0.41 | 154 |
| Example 48-Isomer 1 | >1000 | 62 | |
| Example 48-Isomer 2 | 12 | 0.28 | 65 |
| Example 49 | 25 | 0.54 | 103 |
| Example 50 | 17 | 0.52 | 193 |
| Example 51 | 10 | 0.31 | 142 |
| Example 52 | 116 | 1.7 | 235 |
| Example 53 | 7.4 | 0.31 | 33 |
| Example 54 | 2.2 | 0.37 | 32 |
| Example 55 | 119 | 2.9 | 332 |
| Example 56 | 12 | 0.41 | 109 |
| Example 57 | 4.6 | 0.28 | 294 |

As demonstrated by the data in Table 2, the inventors surprisingly and unexpectedly discovered that the exemplary compounds in Table 2 modulate or inhibit the activity of KRAS G12D.

A number of references have been cited, the disclosures of which are incorporated herein by reference in their entirety.

1. A compound having Formula (I): 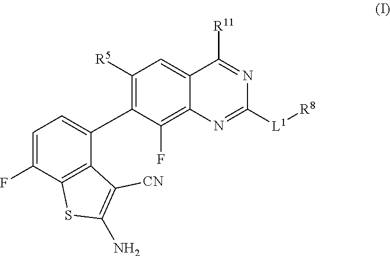
or a pharmaceutically acceptable salt, tautomer, isotopologue, stereoisomer, or prodrug thereof, wherein:
R⁵ is H, —Cl, —CF₃, or unsubstituted C₁₋₄alkyl;
R¹¹ is 3,8-diazabicyclo[3.2.1]oct-3-yl, 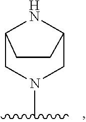 [SMILES: *N1CC2CCC(C1)N2]
3,8-diazabicyclo[3.2.1]oct-8-yl, 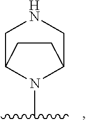 [SMILES: *N1C2CCC1CNC2]
3-oxa-7,9-diazabicyclo[3.3.1]non-9-yl, 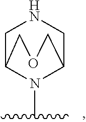 [SMILES: *N1C2CNCC1COC2]
3-oxa-7,9-diazabicyclo[3.3.1]non-7-yl, or 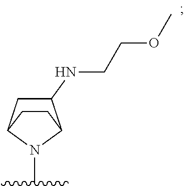 [SMILES: *N1C2CCC1C(NCCOC)C2]
and
L¹ is —O— or a bond;
when R⁵ is H, —CF₃, or unsubstituted C₁₋₄alkyl,
R⁸ is unsubstituted or substituted alkyl, unsubstituted or substituted cycloalkyl, unsubstituted or substituted cycloalkylalkyl, unsubstituted or substituted aryl, unsubstituted or substituted aralkyl, unsubstituted or substituted heterocyclyl, unsubstituted or substituted heterocyclylalkyl, unsubstituted or substituted heteroaryl, unsubstituted or substituted heteroaralkyl, unsubstituted or substituted spirocyclic ring, or unsubstituted or substituted -alkyl-spirocyclic ring;
when R⁵ is —Cl,
R⁸ is 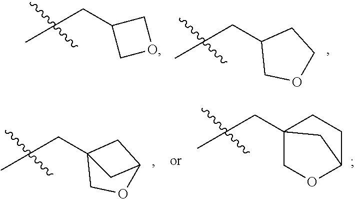
and
R⁸ is optionally substituted.

2. The compound of claim 1, wherein L¹ is —O—.

3. The compound of claim 2, wherein
R⁵ is —CF₃;
R¹¹ is 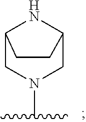 [SMILES: *N1CC2CCC(C1)N2]
R⁸ is unsubstituted or substituted heterocyclylalkyl.

4. The compound of claim 3, wherein the heterocyclylalkyl comprises at least one oxygen.

5. The compound of claim 4, wherein R⁸ is -L^{8a}R^{8a};
L⁸a is —(CH₂)ₙ—;
n is an integer of 1, 2, or 3;
R^{8a} is oxetanyl, tetrahydrofuryl, tetrahydro-2H-pyranyl, dihydro-2H-pyranyl, oxabicyclo[2.1.1]hexyl, oxabicyclo[2.2.1]heptyl, oxaspiro[3.3]heptyl, oxabicyclo[3.2.1]octyl, oxabicyclo[2.2.2]octyl, oxaspiro[3.5]nonyl, or oxaspiro[3.4]octyl;
R^{8a} is optionally substituted.

6. The compound of claim 5, wherein R⁸ is

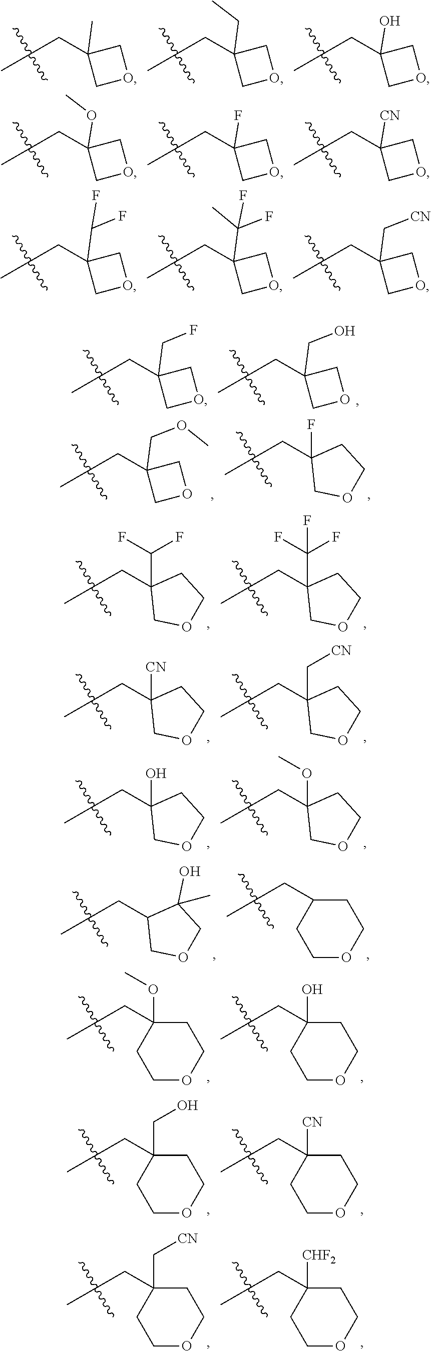

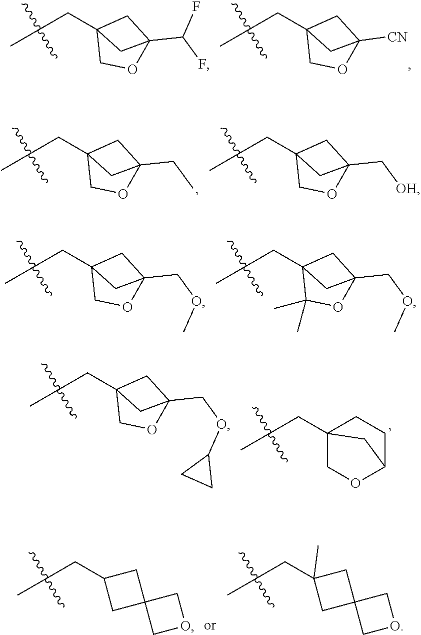

7. The compound of claim 3, wherein the heterocyclylalkyl comprises at least one nitrogen.

8. The compound of claim 7, wherein R⁸ is -L^{8a}R^{8a};
L⁸a is —(CH₂)ₙ—;
n is an integer of 1, 2, or 3;
R^{8a} is azetidyl, pyridyl, isoxazolyl, oxazolyl, dihydro-2H-pyranyl, tetrahydro-2H-pyranyl, pyrrolidinonyl, azaspiro[3.3]heptyl, azabicyclo[2.1.1]hexyl, pyrrolidyl, 1H-pyrazolyl; and
R^{8a} is optionally substituted.

9. The compound of claim 8, wherein R⁸ is

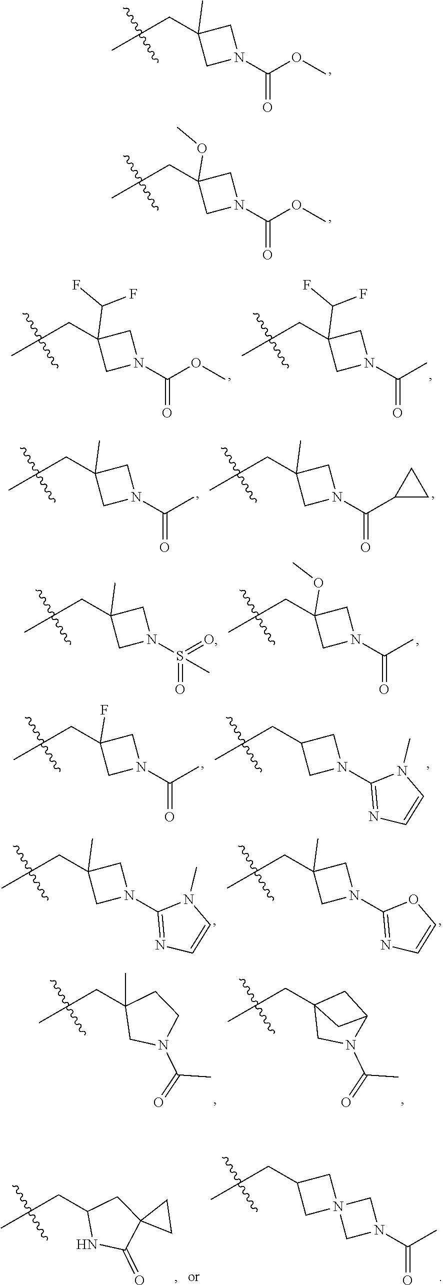

10. The compound of claim 1, wherein L¹ is a bond;
R⁵ is —CF₃;
R¹¹ is 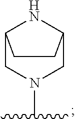 [SMILES: *N1CC2CCC(C1)N2]
R⁸ is unsubstituted or substituted heterocyclyl, unsubstituted or substituted heterocyclylalkyl, unsubstituted or substituted cycloalkyl, or unsubstituted or substituted cycloalkylalkyl.

11. The compound of claim 10, wherein R⁸ is -L^{8a}-R^{8a};
L^{8a} is —(CH₂)ₙ—;
n is an integer of 0, 1, 2, or 3;
R^{8a} is cyclopropyl, cyclobutyl, cyclopentyl, cyclohexyl, cycloheptyl, or azetidyl; and
R^{8a} is optionally substituted.

12. The compound of claim 11, wherein
R⁸ is

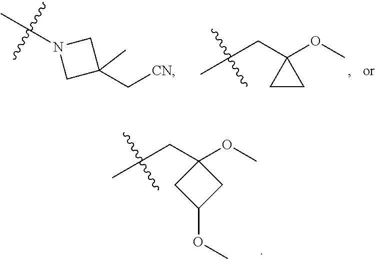

13. The compound of claim 1, wherein
R⁵ is —CF₃;
L¹ is —O—;
R¹¹ is 3,8-diazabicyclo[3.2.1]oct-8-yl, 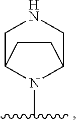 [SMILES: *N1C2CCC1CNC2]
3-oxa-7,9-diazabicyclo[3.3.1]non-9-yl, 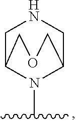 [SMILES: *N1C2CNCC1COC2]
3-oxa-7,9-diazabicyclo[3.3.1]non-7-yl, or 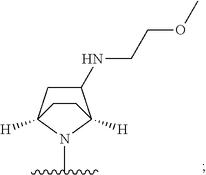 [SMILES: *N1[C@@H]2CC[C@H]1C(NCCOC)C2]
R⁸ is 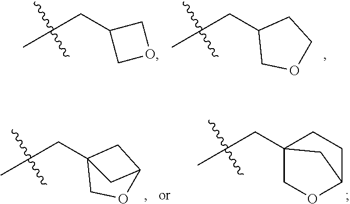 [SMILES: *CC1COC1 ; *CC1CCOC1 ; *CC12COC(C1)C2 ; *CC12CCC(C1)OC2]
and
R⁸ is optionally substituted.

14. The compound of claim 13, wherein the compound is

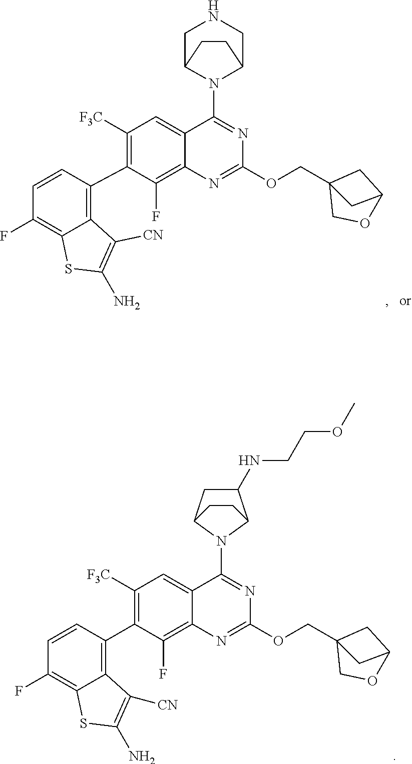 [SMILES: N#Cc1c(N)sc2c(F)ccc(-c3c(C(F)(F)F)cc4c(N5C6CCC5CNC6)nc(OCC56COC(C5)C6)nc4c3F)c12 ; COCCNC1CC2CCC1N2c1nc(OCC23COC(C2)C3)nc2c(F)c(-c3ccc(F)c4sc(N)c(C#N)c34)c(C(F)(F)F)cc12]

15. The compound of claim 1, wherein
R⁵ is —Cl;
L¹ is —O—;
R⁸ is 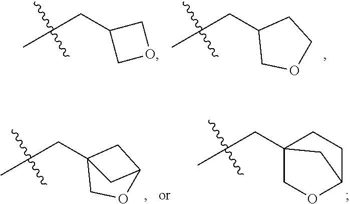 [SMILES: *CC1COC1 ; *CC1CCOC1 ; *CC12COC(C1)C2 ; *CC12CCC(C1)OC2]
and
R⁸ is optionally substituted.

16. The compound of claim 15, wherein the compound is

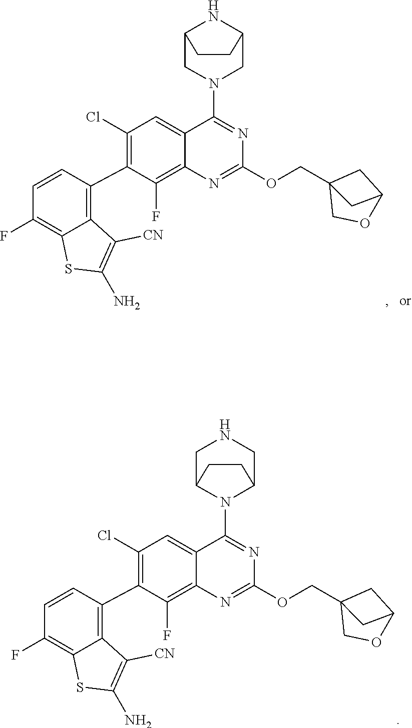 [SMILES: N#Cc1c(N)sc2c(F)ccc(-c3c(Cl)cc4c(N5CC6CCC(C5)N6)nc(OCC56COC(C5)C6)nc4c3F)c12 ; N#Cc1c(N)sc2c(F)ccc(-c3c(Cl)cc4c(N5C6CCC5CNC6)nc(OCC56COC(C5)C6)nc4c3F)c12]

17. The compound of claim 1, wherein
R⁵ is unsubstituted C₁₋₄alkyl;
R¹¹ is 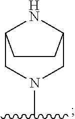 [SMILES: *N1CC2CCC(C1)N2]
L¹ is —O—;
R⁸ is 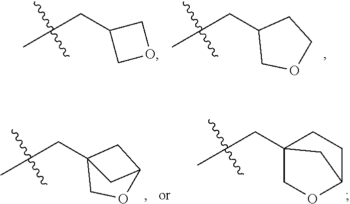 [SMILES: *CC1COC1 ; *CC1CCOC1 ; *CC12COC(C1)C2 ; *CC12CCC(C1)OC2]
and
R⁸ is optionally substituted.

18. The compound of claim 17, wherein the compound is

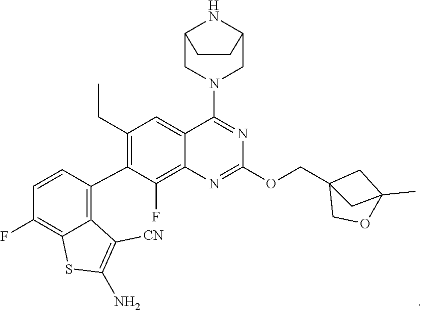 [SMILES: CCc1cc2c(N3CC4CCC(C3)N4)nc(OCC34COC(C)(C3)C4)nc2c(F)c1-c1ccc(F)c2sc(N)c(C#N)c12]

19. The compound of claim 1, wherein
R⁵ is —CF₃;
R¹¹ is 3-oxa-7,9-diazabicyclo[3.3.1]non-9-yl, 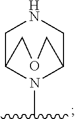 [SMILES: *N1C2CNCC1COC2]
3-oxa-7,9-diazabicyclo[3.3.1]non-7-yl;
L¹ is —O—;
R⁸ is 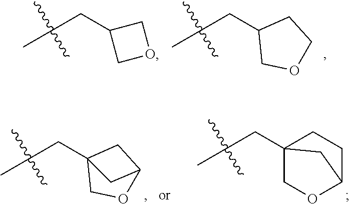 [SMILES: *CC1COC1 ; *CC1CCOC1 ; *CC12COC(C1)C2 ; *CC12CCC(C1)OC2]
and
R⁸ is optionally substituted.

20. The compound of claim 19, wherein the compound is

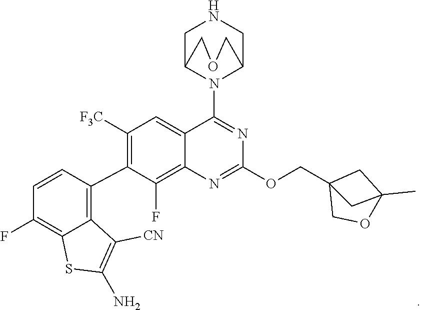 [SMILES: CC12CC(COc3nc(N4C5CNCC4COC5)c4cc(C(F)(F)F)c(-c5ccc(F)c6sc(N)c(C#N)c56)c(F)c4n3)(CO1)C2]

21. The compound of claim 1, wherein the compound is selected from Table 2.

22. A pharmaceutical composition comprising an effective amount of a compound of claim 1, or a pharmaceutically acceptable salt, tautomer, isotopologue, stereoisomer, or prodrug thereof, and a pharmaceutically acceptable carrier, excipient or vehicle.

23. A method for inhibiting the activity of KRAS mutant protein in a cell, comprising contacting said cell with an effective amount of a compound of claim 1, or a pharmaceutically acceptable salt, tautomer, isotopologue, stereoisomer, or prodrug thereof, optionally wherein the KRAS mutant protein is KRAS G12D mutant protein.

24. A method for treatment or prevention of cancer, the method comprising administering to a subject in need thereof an effective amount of claim 1, or a pharmaceutically acceptable salt, tautomer, isotopologue, stereoisomer, or prodrug thereof, optionally wherein the cancer is mediated by KRAS mutation.
